(19) United States
(12) Patent Application Publication
(10) Pub. No.: US 2008/0275073 A1
(43) Pub. Date: Nov. 6, 2008

(54) CRYSTALLINE FORMS AND POLYMORPHS OF N-(3-(DIMETHYLAMINO)PROPYL)-4-(4-(3-FLUORO-4-METHOXYPHENYL) PYRIMIDIN-2-YLAMINO) BENZENESULFONAMIDE AS PHARMACEUTICALLY ACCEPTABLE SALTS

(75) Inventors: Mahmoud Mirmehrabi, Laval (CA); Sreenivasulu Megati, New City, NY (US); Abdolsamad Tadayon, Kirkland (CA); Subodh Deshmukh, White Plains, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/105,567

(22) Filed: Apr. 18, 2008

Related U.S. Application Data
(60) Provisional application No. 60/925,441, filed on Apr. 20, 2007.

(51) Int. Cl. A61K 31/505; C07D 239/42

(52) U.S. Cl. 514/275; 544/332

(57) ABSTRACT

The present invention relates to one or more crystalline forms and polymorphs of compounds of formula I:

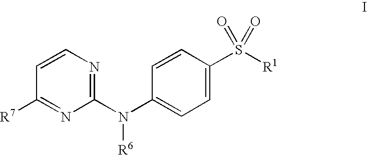

in the form of pharmaceutically acceptable salts. The present invention is directed to crystalline polymorphs and forms of specific anilino-pyrimidine benzenesulfonamide compounds of formula I as pharmaceutically acceptable salts.

### CROSS REFERENCE TO RELATED APPLICATIONS

This application claims the benefit of priority under 35 U.S.C. §119(e) to U.S. Provisional Patent Application No. 60/925,441 filed on Apr. 20, 2007, which is hereby incorporated by reference in its entirety.

### FIELD OF THE INVENTION

The present invention relates to anilino-pyrimidine benzenesulfonamide analogs that are useful for inhibiting protein kinase activity. The invention is directed to one or more crystalline forms and polymorphs of substituted anilino-pyrimidine benzenesulfonamides as pharmaceutically acceptable salts and pharmaceutical compositions and formulations that include the one or more crystalline forms and polymorphs. The invention is also directed to methods for preparing one or more crystalline forms and polymorphs of substituted anilino-pyrimidine benzenesulfonamides as pharmaceutically acceptable salts, methods for preparing pharmaceutical compositions and formulations that include the one or more crystalline forms and polymorphs and methods for treating mammalians and inhibiting abnormal growth of certain cell types. The one or more crystalline forms and polymorphs of certain anilino-pyrimidine benzenesulfonamide analogs inhibit action of a certain protein kinase known as IκB kinase (“IKK”) complex, thereby inhibiting abnormal growth of certain cell types.

### BACKGROUND OF THE INVENTION

Nuclear factor-KB (NF-κB) is a transcriptional factor that regulates the expression of important genes related to cell survival. Activation of NF-κB is central to inflammatory response because it regulates the expression of pro-inflammatory cytokines such as tumor necrosis factor-α (TNF-α). TNF-α not only induces inflammation, but also acts as a survival factor for many cancers and can stimulate the production of angiogenic factors. TNF-α has been found in ovarian, breast, prostate, bladder, and colorectal cancer as well as in lymphomas and leukemias. The role of NF-κB in cancer has been further illuminated by research showing that NF-κB promotes tumorigenesis by suppressing apoptosis and stimulating cell proliferation. Haefner, B. (2002) “NF-κB: arresting a major culprit in cancer,” Drug Discovery Today, 7, 653-663. Because of the role of NF-κB in tumorigenesis and inflammation, NF-κB inhibitors may prove useful as anti-cancer and anti-inflammation therapeutic agents.

The primary form of NF-κB is retained in the cytoplasm of resting cells by IκB, an inhibitor of NF-κB. NF-κB is activated by stimulation of a cellular kinase complex known as IκB kinase (“IKK”) complex, comprising subunits IKKα, β, and γ. Upon stimulation by, for example, a toxin, a cytokine (such as TNF-α), or ionizing radiation, IKK phosphorylates IκB and triggers ubiquitination-dependent degradation through the proteasome pathway. With IκB destroyed, NF-κB is free to enter the nucleus and activate transcription. Hu, M. (2004) “IκB Kinase Promotes Tumorigenesis through Inhibition of Forkhead FOXO3a,” Cell, 117, 225-237. Haefner, B. (2002) “NF-κB: arresting a major culprit in cancer,” Drug Discovery Today, 7, 653-663.

Aberrant expression of IKK has been correlated with activation of NF-κB and, in turn, tumorigenesis and cell proliferation. High IKK levels may also promote tumorigenesis by negatively regulating other transcription factors, such as FOXO factors. Hu, M. (2004) “IκB Kinase Promotes Tumorigenesis through Inhibition of Forkhead FOXO3a,” Cell, 117, 225-237. Thus, inhibiting IKK may inhibit cell proliferation and tumorigenesis.

The crystalline form of a particular drug as a salt, a hydrate, a solvate, and/or any polymorphs thereof is often one important determinant of the drug's ease of preparation, stability, solubility, storage stability, ease of formulation and in-vivo pharmacology. Different crystalline forms of the same composition, known as polymorphs, occur when a composition crystallizes in different lattice arrangements or where solvent molecules including, but not limited to, water molecules are incorporated into the crystalline lattice, resulting in solids with different thermodynamic properties and stabilities specific to the particular form of the drug. It is entirely possible that one crystalline form is preferable over another where certain aspects such as ease of preparation, stability, and like parameters are deemed to be critical. Similarly, greater solubility and/or superior pharmacokinetics may be desired characteristics.

Certain substituted anilino-pyrimidine benzenesulfonamide compounds have been shown to inhibit inappropriately high kinase activity, as disclosed in U.S. Pat. No. 6,048,866. One limitation of such anilino-pyrimidine benzenesulfonamide compounds is that they are not water soluble in a free base form. There is a need for crystalline, water-soluble forms and polymorphs of substituted anilino-pyrimidine benzenesulfonamide compounds as pharmaceutically acceptable salts that selectively inhibit kinase activity and corresponding new methods for preparing and manufacturing such crystalline forms and polymorphs. The present compositions fulfill this need, including IKK inhibitors. Crystalline forms and polymorphs of the invention are useful in the treatment of conditions including, but not limited to for example, polycystic kidney disease, colonic polyps, cancer, and stroke in mammals.

Because improved drug formulations showing, for example, better bioavailability or better stability are progressively achieved, there is an ongoing need for new or high purity crystalline forms and polymorphs of existing drug molecules. One or more crystalline forms and polymorphs of specific anilino-pyrimidine benzenesulfonamide compounds and pharmaceutically acceptable salts thereof are described, characterized and claimed herein.

### SUMMARY OF THE INVENTION

Accordingly, the invention provides crystalline forms and polymorphs of compounds of formula I:

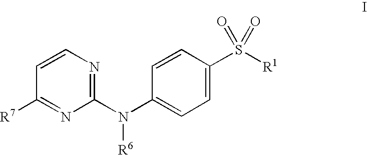

as pharmaceutically acceptable salts, wherein R¹ is —NR²R³, wherein R² and R³ are independently selected from the group consisting of: C₁-C₅ substituted alkyl, C₂-C₅ substituted alkenyl, C₂-C₅ substituted alkynyl, C₂-C₅ substituted aryl, C₁-C₅ substituted heteroaryl, hydroxyl, C₁-C₅ substituted alkoxy, C₁-C₅ substituted alkylamino, C₁-C₅ substituted arylamino, C₁-C₅ substituted heteroarylamino, —NCOR⁴, —COR⁴, —CONR²R³, SO₂R⁵, C₄-C₁₀ substituted 3 to 10 membered cyclic amines containing 0 to 3 heteroatoms; R⁴ and R⁵ are independently selected from the group consisting of hydrogen, methyl, trifluoromethyl, substituted alkyl, substituted aryl, and substituted heteroaryl; R⁶ is selected from the group consisting of hydrogen, C₁-C₅ substituted alkyl, C₁-C₅ substituted alkylcarbonyl, and C₁-C₅ substituted alkoxycarbonyl; and wherein R⁷ is a substituted phenyl selected from the group consisting of: 2,4-substituted phenyl, 3,4-substituted phenyl, 4,5-substituted phenyl and 4,6-substituted phenyl, the substituted phenyl having substituents selected from the group consisting of: C₁-C₅ alkyl, F, Cl, Br, I, C₁-C₅ alkoxy, C₁-C₅ alkylamine, C₁-C₅ alkylamino, C₁-C₅ amide, C₁-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl. The pharmaceutically acceptable salts of the compounds of formula I includes any pharmaceutically acceptable solvates and hydrates of the salts.

The invention also provides crystalline forms and polymorphs of compounds of formula II:

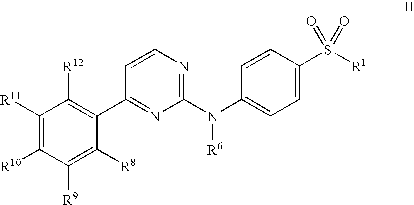

in the form of a pharmaceutically acceptable salt; wherein R¹ is —NR²R³, wherein R² and R³ are independently selected from the group consisting of: C₁-C₅ substituted alkyl, C₂-C₅ substituted alkenyl, C₂-C₅ substituted alkynyl, C₂-C₅ substituted aryl, C₁-C₅ substituted heteroaryl, hydroxyl, C₁-C₅ substituted alkoxy, C₁-C₅ substituted alkylamino, C₁-C₅ substituted arylamino, C₁-C₅ substituted heteroarylamino, —NCOR⁴, —COR⁴, —CONR²R³, SO₂R⁵, C₄-C₁₀ substituted 3 to 10 membered cyclic amines containing 0 to 3 heteroatoms; R⁴ and R⁵ are independently selected from the group consisting of hydrogen, methyl, trifluoromethyl, substituted alkyl, substituted aryl, and substituted heteroaryl; R⁶ is selected from the group consisting of hydrogen, C₁-C₅ substituted alkyl, C₁-C₅ substituted alkylcarbonyl, and C₁-C₅ substituted alkoxycarbonyl; and wherein R³-R¹² are independently selected from the group consisting of: C₁-C₅ alkyl, F, Cl, Br, I, C₁-C₅ alkoxy, C₁-C₅ alkylamine, C₁-C₅ alkylamino, C₁-C₅ amide, C₁-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl. The pharmaceutically acceptable salt of the compounds of formula II includes any pharmaceutically acceptable solvates and hydrates of the salt.

The invention further provides pharmaceutical compositions and formulations including one or more of the crystalline forms and polymorphs of the compounds of formulas I or II as pharmaceutically acceptable salts. The invention also provides methods for synthesizing, characterizing and manufacturing pharmaceutical compositions and formulations including one or more of the crystalline forms and polymorphs of the compounds of formula I as pharmaceutically acceptable salts.

The invention also provides pharmaceutical compositions comprising one or more of the crystalline forms and polymorphs of the compounds of formulas I or II as pharmaceutically acceptable salts or their combination with other kinase-inhibiting pharmaceutical compositions or chemotherapeutic agents, and a pharmaceutically acceptable carrier.

The invention also provides methods for inhibiting kinase activity in a cell comprising contacting a cell with one or more crystalline forms and polymorphs of compounds of formulas I or II as pharmaceutically acceptable salts, whereby the compound inhibits kinase activity.

The present invention also provides pharmaceutical compositions comprising one or more crystalline forms and polymorphs of compounds of formulas I or II as pharmaceutically acceptable salts and a pharmaceutically acceptable carrier. In another embodiment, the present invention provides a method of inhibiting kinase action, especially IKK, in a cell by providing a compound of the present invention. The present invention also provides a method of inhibiting kinase activity, especially IKK, in a mammal, especially a human, by administering to a patient a compound or pharmaceutical composition of the present invention. The present invention also provides a method of treating a kinase-dependent condition, especially inflammation or cancer, by administering to a patient one or more crystalline forms and polymorphs of compounds of formula I or II as pharmaceutically acceptable salts.

The present invention provides a method of treating mammalian diseases associated with NF-κB activation by administering to a patient, one or more crystalline forms and polymorphs of compounds of formulas I or II as pharmaceutically acceptable salts in a pharmaceutical composition or formulation.

The present invention provides methods of treating cancer; inflammatory or autoimmune conditions; cardiovascular, metabolic, or ischemic conditions; infectious diseases, particularly viral infections; as well as pre- or post-menopausal conditions, particularly osteoporosis, by administering to a patient, one or more crystalline forms and polymorphs of formulas I or II as pharmaceutically acceptable salts in a pharmaceutical composition or formulation.

The present invention also provides methods further comprising administering an inhibitor of a protein kinase of the NF-κB pathway to a patient, the inhibitor comprising one or more crystalline forms and polymorphs of formulas I or II as pharmaceutically acceptable salts in a pharmaceutical composition or formulation.

The present invention also provides methods further comprising administering an inhibitor of a protein kinase of the NF-κB pathway to a patient, the inhibitor comprising one or more crystalline forms and polymorphs of formula I or II as pharmaceutically acceptable salts in a pharmaceutical composition or formulation and an additional inhibitor of a protein kinase of the NF-κB pathway.

### BRIEF DESCRIPTION OF THE FIGURES

FIG. 1 depicts a polycrystalline X-ray diffraction (XRD) pattern of one form of N-(3-(dimethylamino)propyl)-4-(4-(3-fluoro-4-methoxyphenyl)pyrimidin-2-ylamino)benzenesulfonamide as a mono-succinate salt (Form I), where the diffraction angle (2θ) ranges from 0-30 degrees with a step of 0.01 degrees.

FIG. 2a depicts a nuclear magnetic resonance spectrum of N-(3-(dimethylamino)propyl)-4-(4-(3-fluoro-4-methoxyphenyl)pyrimidin-2-ylamino)-benzenesulfonamide as a free base.

FIG. 2b depicts a nuclear magnetic resonance spectrum of N-(3-(dimethylamino)propyl)-4-(4-(3-fluoro-4-methoxyphenyl)pyrimidin-2-ylamino)-benzenesulfonamide as a mono-succinate salt.

FIG. 3 depicts a DSC scan of N-(3-(dimethylamino)propyl-4-(4-(3-fluoro-4-methoxyphenyl)pyrimidin-2-ylamino)benzenesulfonamide as a mono-succinate salt.

FIG. 4 depicts a TGA thermogram of the mono-succinate salt of N-(3-(dimethylamino)propyl)-4-(4-(3-fluoro-4-methoxyphenyl)pyrimidin-2-ylamino)-benzenesulfonamide.

FIG. 5 depicts a polycrystalline XRD pattern of one polymorph of N-(3-(dimethylamino)propyl)-4-(4-(3-fluoro-4-methoxyphenyl)pyrimidin-2-ylamino)-benzenesulfonamide as a hemi-succinate salt (Form I), where the diffraction angle (2θ) ranges from 0-30 degrees with a step of 0.01 degrees.

FIG. 6 depicts a nuclear magnetic resonance spectrum of N-(3-(dimethylamino)propyl)-4-(4-(3-fluoro-4-methoxyphenyl)pyrimidin-2-ylamino)benzenesulfonamide as a hemi-succinate salt (Form I).

FIG. 7 depicts a DSC scan of N-(3-(dimethylamino)propyl)-4-(4-(3-fluoro-4-methoxyphenyl)pyrimidin-2-ylamino)benzenesulfonamide as a hemi-succinate salt (Form I).

FIG. 8 depicts a TGA thermogram of N-(3-(dimethylamino)propyl)-4-(4-(3-fluoro-4-methoxyphenyl)pyrimidin-2-ylamino)-benzenesulfonamide as a hemi-succinate salt (Form I).

FIG. 9 depicts a polycrystalline XRD pattern of a different polymorph of N-(3-(dimethylamino)propyl)-4-(4-(3-fluoro-4-methoxyphenyl)pyrimidin-2-ylamino)-benzenesulfonamide as a hemi-succinate salt (Form II), where the diffraction angle (2θ) ranges from 0-30 degrees with a step of 0.01 degrees.

FIG. 10 depicts a DSC scan of the Form II polymorph of N-(3-(dimethylamino)propyl)-4-(4-(3-fluoro-4-methoxyphenyl)pyrimidin-2-ylamino)benzenesulfonamide as a hemi-succinate salt (Form II).

FIG. 11 depicts a TGA thermogram of N-(3-(dimethylamino)propyl)-4-(4-(3-fluoro-4-methoxyphenyl)pyrimidin-2-ylamino)-benzenesulfonamide as a hemi-succinate salt (Form II).

FIG. 12 depicts a polycrystalline XRD pattern of another polymorph of N-(3-(dimethylamino)propyl)-4-(4-(3-fluoro-4-methoxyphenyl)pyrimidin-2-ylamino)benzenesulfonamide as a hemi-succinate salt (Form II), where the diffraction angle (2θ) ranges from 0-30 degrees with a step of 0.01 degrees.

FIG. 13 depicts a polycrystalline XRD pattern of another polymorph of N-(3-(dimethylamino)propyl)-4-(4-(3-fluoro-4-methoxyphenyl)pyrimidin-2-ylamino)benzenesulfonamide as a hemi-succinate salt (Form IV), where the diffraction angle (2θ) ranges from 0-30 degrees with a step of 0.01 degrees.

FIG. 14 depicts a polycrystalline XRD pattern of yet another polymorph of N-(3-(dimethylamino)propyl)-4-(4-(3-fluoro-4-methoxyphenyl)pyrimidin-2-ylamino)benzenesulfonamide as a hemi-succinate salt (Form V), where the diffraction angle (2θ) ranges from 0-30 degrees with a step of 0.01 degrees.

FIG. 15 depicts a polycrystalline XRD pattern of one polymorph of N-(3-(dimethylamino)propyl)-4-(4-(3-fluoro-4-methoxyphenyl)pyrimidin-2-ylamino)-benzenesulfonamide as an acetate salt (Form I), where the diffraction angle (2θ) ranges from 0-30 degrees with a step of 0.01 degrees.

FIG. 16 depicts a DSC scan of N-(3-(dimethylamino)propyl)-3-(3-fluoro-4-methoxyphenyl)pyrimidin-2-ylamino)benzenesulfonamide as an acetate salt (Form II).

FIG. 17 depicts a TGA thermogram of N-(3-(dimethylamino)propyl)-4-(4-(3-fluoro-4-methoxyphenyl)pyrimidin-2-ylamino)-benzenesulfonamide as an acetate salt (Form II).

FIG. 18 depicts a polycrystalline XRD pattern of a different polymorph of N-(3-(dimethylamino)propyl)-4-(4-(3-fluoro-4-methoxyphenyl)pyrimidin-2-ylamino)-benzenesulfonamide as an acetate salt (Form II), where the diffraction angle (2θ) ranges from 0-30 degrees with a step of 0.01 degrees.

FIG. 19 depicts a polycrystalline XRD pattern of another polymorph of N-(3-(dimethylamino)propyl)-4-(4-(3-fluoro-4-methoxyphenyl)pyrimidin-2-ylamino)benzenesulfonamide as an acetate salt (Form II), where the diffraction angle (2θ) ranges from 0-30 degrees with a step of 0.01 degrees.

FIG. 20 depicts a polycrystalline XRD pattern of another polymorph of N-(3-(dimethylamino)propyl)-4-(4-(3-fluoro-4-methoxyphenyl)pyrimidin-2-ylamino)benzenesulfonamide as an acetate salt (Form IV), where the diffraction angle (2θ) ranges from 0-30 degrees with a step of 0.01 degrees.

FIG. 21 depicts a polycrystalline XRD pattern of yet another polymorph of N-(3-(dimethylamino)propyl)-4-(4-(3-fluoro-4-methoxyphenyl)pyrimidin-2-ylamino)benzenesulfonamide as an acetate salt (Form V), where the diffraction angle (2θ) ranges from 0-30 degrees with a step of 0.01 degrees.

FIG. 22 depicts a polycrystalline XRD pattern of yet another different polymorph of N-(3-(dimethylamino)propyl)-4-(4-(3-fluoro-4-methoxyphenyl)pyrimidin-2-ylamino)benzenesulfonamide as an acetate salt (Form VI), where the diffraction angle (2θ) ranges from 0-30 degrees with a step of 0.01 degrees.

FIG. 23 depicts a polycrystalline XRD pattern of one polymorph of N-(3-(dimethylamino)propyl)-4-(4-(3-fluoro-4-methoxyphenyl)pyrimidin-2-ylamino)-benzenesulfonamide as a D-glucoronate salt (Form I), where the diffraction angle (2θ) ranges from 0-30 degrees with a step of 0.01 degrees.

FIG. 24 depicts a DSC scan of N-(3-(dimethylamino)propyl)-4-(3-fluoro-4-methoxyphenyl)benzenesulfonamide as a D-glucoronate salt (Form II).

FIG. 25 depicts a TGA thermogram of N-(3-(dimethylamino)propyl)-4-(3-fluoro-4-methoxylphenyl)benzenesulfonamide as a D-glucoronate salt (Form II).

FIG. 26 depicts a polycrystalline XRD pattern of a different polymorph of N-(3-(dimethylamino)propyl)-4-(4-(3-fluoro-4-methoxyphenyl)pyrimidin-2-ylamino)-benzenesulfonamide as a D-glucoronate salt (Form II), where the diffraction angle (2θ) ranges from 0-30 degrees with a step of 0.01 degrees.

FIG. 27 depicts a polycrystalline XRD pattern of another polymorph of N-(3-(dimethylamino)propyl)-4-(4-(3-fluoro-4-methoxyphenyl)pyrimidin-2-ylamino)benzenesulfonamide as a D-glucoronate salt (Form II), where the diffraction angle (2θ) ranges from 0-30 degrees with a step of 0.01 degrees.

FIG. 28 depicts a polycrystalline XRD pattern of yet another different polymorph of N-(3-(dimethylamino)propyl)-4-(4-(3-fluoro-4-methoxyphenyl)pyrimidin-2-ylamino)benzenesulfonamide as a D-glucoronate salt (Form IV), where the diffraction angle (2θ) ranges from 0-30 degrees with a step of 0.01 degrees.

### DETAILED DESCRIPTION OF THE INVENTION

For convenience, certain terms employed in the specification, examples, and appended claims are collected here.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 6 or fewer carbon atoms in its backbone. The term “alkyl” can be used alone or as part of a chemical name as in for example, “trialkylorthoformate”. The terms “alkenyl” and “alkynyl” refer to unsaturated aliphatic groups analogous in length and possible substitution to the alkyls described above, but which contain at least one double or triple carbon-carbon bond, respectively. Analogous substitutions can be made to alkenyl and alkynyl groups to produce, for example, alkenylamines, alkynylamines, alkenylamides, alkynylamides, alkenylimines, alkynylimines, thioalkenyls, thioalkynyls, carbonyl-substituted alkenyls or alkynyls, alkenoxyls, alkynoxyls, metalloalkenyls and metalloalkynyls.

The term “alkoxy” as used herein refers to an alkyl group, as defined above, having an oxygen radical attached thereto. Representative alkoxyl groups include methoxy, ethoxy, propyloxy, tert-butoxy and the like. The term alkoxy can be used alone or as part of a chemical name as in for example, “alkoxy-enaminonitrile”. Alkoxy also means a group —OR, wherein R is an alkyl, alkenyl, or alkynyl group which can optionally be substituted with one or more functional groups. Hydroxy means —OH. Carbonyl means carbon bonded to oxygen with a double bond, i.e., C═O. Amino means the —NH₂ group.

The term “aryl” as used herein includes 4-, 5-, 6-, 7- and 10-membered carbocyclic single ring or fused multiple ring aromatic groups, which may be substituted or unsubstituted. Accordingly the term “phenyl” refers to a 6-membered carbocyclic single ring, which is partially substituted with substituents and other chemical groups at positions 1-5. The term “heteroaryl” refers to a 4 to 10 aromatic membered ring structure, which ring structure includes one to four heteroatoms. Heteroaryls include, but are not limited to, pyrrolidine, oxolane, thiolane, piperidine, piperazine, pyrrole, furan, thiophene, imidazole, oxazole, thiazole, triazole, pyrazole, pyridine, pyrazine, pyridazine and pyrimidine, and morpholine.

The term “heteroatom” as used herein means an atom of any element other than carbon or hydrogen. Suitable examples of heteroatoms include, but are not limited to, nitrogen, oxygen, sulfur, phosphorus, and selenium.

The term “halogen” refers to an atom of fluorine, chlorine, bromine, or iodine.

As used herein, the term “substituted” is contemplated to include all permissible substituents of organic compounds. Typically, suitable substituents of organic compounds include acyclic and cyclic, branched and unbranched, carbocyclic and heterocyclic, aromatic and nonaromatic substituents of organic compounds as well as inorganic substituents such as halogen or amino. The substituents can be one or more and the same or different for appropriate organic compounds. For purposes of this invention, the heteroatoms such as nitrogen may have hydrogen substituents, halogen substituents and/or any suitable substituents of organic compounds described herein which satisfy the valencies of the heteroatoms. This invention is not intended to be limited in any manner by the suitable substituents of organic compounds.

Hydrates are solid compounds containing water molecules combined in a definite ratio as an integral part of the crystalline compound. Examples of hydrates include, but are not limited to for example: hemihydrate, monohydrate, dihydrate, trihydrate, quadrahydrate, pentahydrate and hexahydrate. Hydrates also are intended to include solids compounds containing water molecules combined in a non-stoichiometric ratio as an integral part of the crystalline compound.

Solvates are solid compounds containing solvent molecules combined in a definite ratio as an integral part of the crystalline compound. Solvates also are intended to include solid compounds containing solvent molecules combined in a non-stoichiometric ratio as an integral part of the crystalline compound.

Alkylamino means the —NHR or NR where R is a C₁-C₄ alkyl group, which optionally may be substituted.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The present invention is directed to one or more crystalline forms and polymorphs of certain substituted anilino-pyrimidine benzenesulfonamide compounds in the form of corresponding pharmaceutically acceptable salts, pharmaceutical compositions including the one or more crystalline forms and polymorphs of the compounds as corresponding pharmaceutically acceptable salts, pharmaceutical formulations including the one or more crystalline forms and polymorphs of the compounds as corresponding pharmaceutically acceptable salts, and methods of preparing and manufacturing one or more crystalline forms and polymorphs of certain substituted anilino-pyrimidine benzenesulfonamide compounds in the form of corresponding pharmaceutically acceptable salts. The invention also provides methods for converting one crystalline form or polymorph of a certain substituted aniline-pyrimidine benzenesulfonamide compound in the form of a pharmaceutically acceptable salt to one or more different polymorphs of the certain substituted aniline-pyrimidine benzenesulfonamide compound in the form of the corresponding pharmaceutically acceptable salt.

In one embodiment, the certain substituted aniline-pyridine benzenesulfonamide are provided as compounds of formula I:

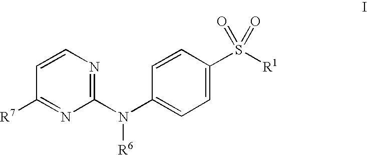

wherein, R¹ is —NR²R³, wherein R² and R³ are independently selected from the group consisting of: C₁-C₅ substituted alkyl, C₂-C₅ substituted alkenyl, C₂-C₅ substituted alkynyl, C₂-C₅ substituted aryl or phenyl, C₁-C₅ substituted heteroaryl, hydroxyl, C₁-C₅ substituted alkoxy, C₁-C₅ substituted alkylamino, C₁-C₅ substituted arylamino, C₁-C₅ substituted heteroarylamino, —NCOR⁴, —COR⁴, —CONR²R³, SO₂R⁵, C₄-C₁₀ substituted 3 to 10 membered cyclic amines containing 0 to 3 heteroatoms; R⁴ and R⁵ are independently selected from the group consisting of hydrogen, methyl, trifluoromethyl, substituted alkyl, substituted aryl, and substituted heteroaryl; R⁶ is selected from the group consisting of hydrogen, methyl, C₂-C₅ substituted alkyl, C₁-C₅ substituted alkylcarbonyl, and C₁-C₅ substituted alkoxycarbonyl; and wherein R⁷ is a substituted phenyl selected from the group consisting of: 2,4-substituted phenyl, 3,4-substituted phenyl, 4,5-substituted phenyl and 4,6-substituted phenyl, the substituted phenyl having substituents selected from the group consisting of: C₁-C₅ alkyl, F, Cl, Br, I, C₁-C₅ alkoxy, C₁-C₅ alkylamine, C₁-C₅ alkylamino, C₁-C₅ amide, C₁-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl.

According to one embodiment, R⁷ is a substituted 5 or 6 membered heteroaryl ring with 1 to 4 heteroatoms, provided that the heteroaryl ring is not pyridine, furan, isoxazole, pyrazole, triazole, imidazole, or thiazole; a benzene ring fused to a 4 to 8 membered ring containing 0 to 4 heteroatoms, interrupted by 0 to 2 of the groups C═O, SO, or SO₂, and optionally substituted; an optionally substituted monocyclic or polycyclic ring containing 0 to 4 heteroatoms; optionally substituted alkenyl; optionally substituted alkynyl; —NR²R³; —COOR⁴; —CONR²R³; and —SO₂R⁵.

In a separate embodiment, the certain substituted aniline-pyridine benzenesulfonamide are provided as compounds of formula II:

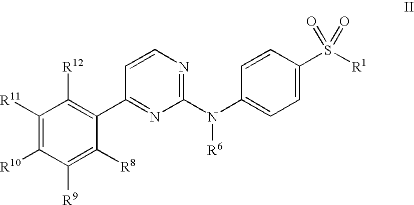

wherein, R¹-R⁶ is defined as above and wherein a pyrimidin-2-yl-substituted phenyl comprises substituents R⁸-R¹² are independently selected from the group consisting of: C₁-C₅ alkyl, F, Cl, Br, I, C₁-C₅ alkoxy, C₁-C₅ alkylamine, C₁-C₅ alkylamino, C₁-C₅ amide, C₂-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl.

In a separate embodiment, the certain substituted anilino-pyrimidine benzenesulfonamide compounds are provided as compounds formula III:

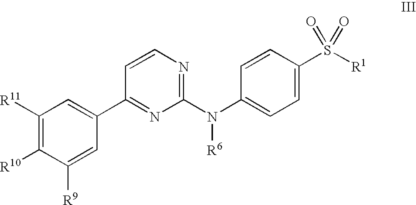

wherein R¹, R⁵ and R⁹-R¹¹ are defined as above.

Methods for synthesizing substituted anilino-pyrimidine benzenesulfonamides, such as N-(3-(dimethylamino)propyl)-4-(4-(3-fluoro-4-methoxyphenyl)pyrimidin-2-ylamino)benzenesulfonamide, are described in PCT Publication WO 2006/044457 A1 and U.S. Pat. No. 6,794,403. According to a separate embodiment, R⁷ is a substituted phenyl group, as described in U.S. Pat. No. 6,794,403, which is used as a substituent for the 2-position of the pyrimidine ring. In one embodiment, the present invention provides methods for preparing a compound of formula I by reacting an enaminone and a guanidine as described in PCT publication WO 2006044457A1. In one embodiment, an enaminone of formula G-1 is reacted with a guanidine of formula G-2 in the presence of 1-methyl-2-pyrrolidinone (NMP). The enaminone G-1 can be prepared by any method known in the art, such as the reaction of an acetyl derivative with an acetal, preferably N,N-dimethylformamide dimethyl acetal, or tert-butoxybis(dimethylamino)methane. The guanidine G-2 can be prepared by reacting an amine of formula G-3 with cyanamide or 1-H-pyrazole-1-carboximidine. Alternatively, the guanidine G-2 can be prepared by reacting a halogenated sulfonamide of formula G-4 with guanidine as described in PCT publication WO2006044457A1. In another embodiment, the SO₂R² group is added after the formation of the pyrimidine. This method includes the steps of: reacting an enaminone G-1 with a guanidine derivative of formula 3-1 and NMP to form a pyrimidine; reacting the pyrimidine with chlorosulfonic acid to form a sulfonyl chloride of formula 3-3; and reacting the sulfonyl chloride 3-3 with an amine having the formula HNR¹R² to form a compound of formula I.

In another embodiment, the present invention provides methods for preparing a compound of formula I by halogen displacement. The reactions can be conducted in a solvent, preferably dioxane. In a preferred embodiment of reactions, R¹ is a substituted phenyl or optionally substituted thienyl group.

In another embodiment, an amine G-3 is reacted with a halogenated pyrimidine of formula G-5 as described in PCT publication WO2006044457A1. Preferably, the halogen of the halogenated pyrimidine is chlorine. For example, the reaction is conducted in the presence of p-toluenesulfonic acid.

In another embodiment, a halogenated sulfonamide of formula G-4 is reacted with a pyrimidine of formula G-6 as described in PCT publication WO200644457A1. For example, the halogen of the halogenated sulfonamide is bromine, the reaction includes a step of adding sodium tert-butoxide (NaOtBu) and the reaction is preferably conducted in the presence of tris(dibenzylideneacetone)dipalladium(0) (Pd₂ dba₃) and 2,2′-bis(diphenylphosphino)-1,1′-binaphthyl (BINAP). In either exemplary embodiments or sequence of the reactions, the process produces a compound of formula I wherein R¹ is NR²R³, and R⁶ is hydrogen.

According to a separate embodiment, R¹ is selected from the group consisting of NR²R³, optionally substituted imidazolyl, and optionally substituted alkyl.

In another embodiment, R¹ is NR²R³, and R² and R³ are independently selected from the group consisting of hydrogen, alkyl, amino and alkylamino (including cyclic amines), alkylhydroxy, alkanoyl, alkoxy, alkoxycarbonyl, carbonyl, carboxyl, aralkyl, optionally substituted phenyl, heteroaryl, and COR⁴ where R⁴ is alkyl or aralkyl. In yet another embodiment, R¹ is NH₂, -(dimethylamino)ethyl, or -(dimethylamino)propyl.

In another embodiment of R¹, R² and R³ are taken together to form an optionally substituted 3 to 12 membered monocyclic or bicyclic ring containing 0 to 4 heteroatoms. In one embodiment, R¹ is an optionally substituted 5 to 6 membered heterocyclic group containing at least one nitrogen atom and 0 to 1 additional heteroatoms. R¹ can be, for example, an optionally substituted morpholinyl group, an optionally substituted piperazinyl group, or an optionally substituted pyrrolidinyl group.

In another embodiment, R¹ is NR²R³, and R¹ is selected from the group of structures listed as Set 2a:

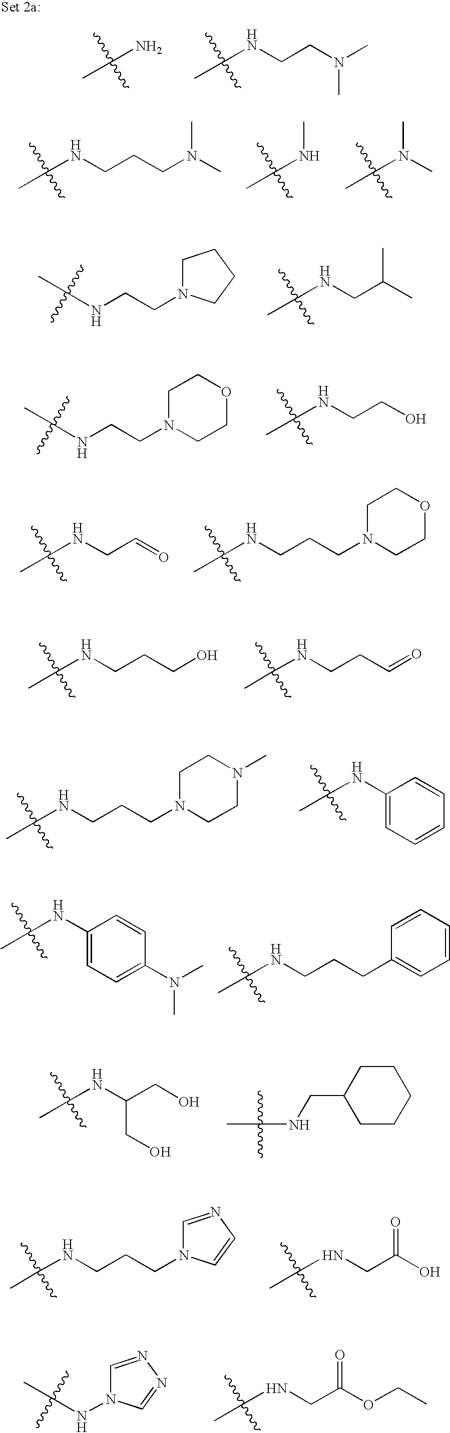

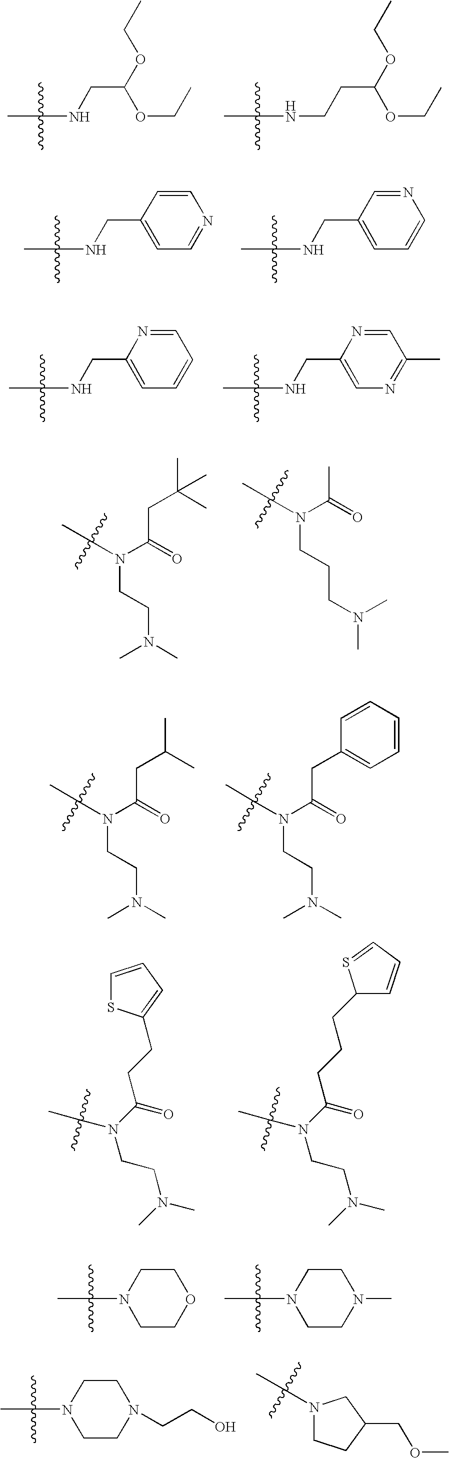

In another embodiment, R¹ is selected from the group of structures listed as Set 2b:

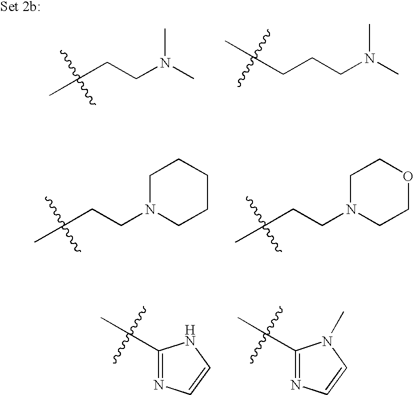

According to a separate embodiment, the pyrimidin-2-yl substituted phenyl group is selected from the group consisting of a para-substituted phenyl, an optionally substituted thienyl, and an optionally benzothiophene, wherein the optional substitution is at least one of C₁-C₅ alkyl, F, Cl, Br, C₁-C₅ alkoxy, amine, C₁-C₅ alkylamino, C₁-C₅ amide, C₂-C₅ ester, or hydroxy, and the C₁-C₅ alkyl, C₁-C₅ alkoxy, C₁-C₅ alkylamino, NH₂ and amide optionally substituted with at least one C₁-C₂ alkyl, C₁-C₄ alkoxy, amine, C₁-C₂ alkylamino, C₁-C₄ amide, C₂-C₄ ester, hydroxy, thienyl, or phenyl.

In one embodiment, the pyrimidin-2-yl substituted phenyl group is a multi-substituted phenyl group substituted at least at the para-position. In another embodiment, the pyrimidin-2-yl substituted phenyl group is a di-substituted phenyl group substituted at least at the para-position. In another embodiment, the pyrimidin-2-yl substituted phenyl group is a di-substituted phenyl group substituted at positions selected from the group consisting of 2,4-, 3,4-, 4,5-, and 4,6-positions, as described in U.S. Pat. No. 6,794,403, which is used a substituent for the 2-position of the pyrimidine ring.

Exemplary substituents for each of R⁸-R¹² include, for example, C₁-C₅ alkyl, F, Cl, Br, I, C₁-C₅ alkoxy, amine, C₁-C₅ alkylamino, C₁-C₅ amide, C₂-C₅ ester, or hydroxy, and the alkyl, alkoxy, alkylamino, NH₂ and amide may optionally be substituted with at least one C₁-C₂ alkyl, C₁-C₄ alkoxy, amine, C₁-C₂ alkylamino, C₁-C₄ amide, C₂-C₄ ester, hydroxy, thienyl, or phenyl. Other exemplary substituents for R³ include, for example, alkoxy, trifluoromethyl, fluoro, hydroxy, and NR²R³ where R² is COR⁴ and R³ is hydrogen.

In one embodiment R⁶ is selected from the group consisting of hydrogen, methyl, alkyl, alkylcarbonyl, or alkoxycarbonyl. In another embodiment, R⁶ is hydrogen or methyl.

According to one embodiment, crystalline forms and polymorphs of the invention also includes any solvates and hydrates of the compounds of formula I described.

Where present, compositions and the one or more crystalline polymorphs of the invention also include isomers either individually or as a mixture, such as enantiomers, diastereomers, and positional isomers.

Exemplary compounds of the present invention in the form of their corresponding free base include the following compounds:

| | |
|---|---|
| 1. | 4-{[4-(4-hydroxyphenyl)pyrimidin-2- |
| | yl]amino}benzenesulfonamide |
| 2. | N-[3-(dimethylamino)propyl]-4-[(4-{4-[2- |
| | (2-thienyl)ethoxy]phenyl}-pyrimidin-2- |
| | yl)amino]benzenesulfonamide |
| 3. | N-(3-dimethylamino)propyl)-4-(4-(3-fluoro-4- |
| | methoxyphenyl)pyrimidin-2- |
| | ylamino)benzenesulfonamide |
| 4. | 4-(4-(4-(2-amino-3-phenylpropoxy)phenyl)pyrimidin-2- |
| | ylamino)benzenesulfonamide |
| 5. | 4-(4-(4-(2-amino-3-methylbutoxy)phenyl)pyrimidin- |
| | 2-ylamino)benzenesulfonamide |
| 6. | N-(3-dimethylamino)propyl)-4-(4-(6-fluoro-4- |
| | methoxyphenyl)pyrimidin-2-ylamino) |
| | benzene sulfonamide |
| 7. | N-(3-dimethylamino)propyl)-4-(4-(2-fluoro-4- |
| | methoxyphenyl)pyrimidin-2-ylamino) |
| | benzene sulfonamide |
| 8. | N-(3-dimethylamino)propyl)-4-(4-(5-fluoro-4- |
| | methoxyphenyl)pyrimidin-2-ylamino) |
| | benzene sulfonamide |
| 9. | N-(3-dimethylamino)propyl)-4-(4-(3-trifluoromethyl- |
| | 4-methoxyphenyl)pyrimidin-2- |
| | ylamino)benzenesulfonamide |

Exemplary crystalline forms and polymorphs of the substituted aniline-pyrimidine benzenesulfonamides in the form of a corresponding pharmaceutically acceptable salt include the following:

| | |
|---|---|
| 10. | N-(3-dimethylamino)propyl)-4-(4-(3-trifluoromethyl-4- |
| | methoxyphenyl)pyrimidin- |
| | 2-ylamino)benzenesulfonamide mono-succinate |
| 11. | N-(3-dimethylamino)propyl)-4-(4-(3-trifluoromethyl-4- |
| | methoxyphenyl)pyrimidin- |
| | 2-ylamino)benzenesulfonamide hemi-succinate (Form I) |
| 12. | N-(3-dimethylamino)propyl)-4-(4-(3-fluoro-4- |
| | methoxyphenyl)pyrimidin-2- |
| | ylamino)benzenesulfonamide hemi-succinate (Form II) |
| 13. | N-(3-dimethylamino)propyl)-4-(4-(3- |
| | fluoro-4-methoxyphenyl)pyrimidin-2- |
| | ylamino)benzenesulfonamide hemi-succinate (Form III) |
| 14. | N-(3-dimethylamino)propyl)-4-(4-(3-fluoro- |
| | 4-methoxyphenyl)pyrimidin-2- |
| | ylamino)benzenesulfonamide hemi-succinate (Form IV) |
| 15. | N-(3-dimethylamino)propyl)-4-(4-(3-fluoro- |
| | 4-methoxyphenyl)pyrimidin-2- |
| | ylamino)benzenesulfonamide hemi-succinate (Form V) |
| 16. | N-(3-dimethylamino)propyl)-4-(4-(3-fluoro- |
| | 4-methoxyphenyl)pyrimidin-2- |
| | ylamino)benzenesulfonamide acetate (Form I) |
| 17. | N-(3-dimethylamino)propyl)-4-(4-(3-fluoro-4- |
| | methoxyphenyl)pyrimidin-2- |
| | ylamino)benzenesulfonamide acetate (Form II) |
| 18. | N-(3-dimethylamino)propyl)-4-(4-(3-fluoro-4- |
| | methoxyphenyl)pyrimidin-2-ylamino) |
| | benzene sulfonamide acetate (Form III) |
| 19. | N-(3-dimethylamino)propyl)-4-(4-(3-fluoro-4- |
| | methoxyphenyl)pyrimidin-2-ylamino) |
| | benzene sulfonamide acetate (Form IV) |
| 20. | N-(3-dimethylamino)propyl)-4-(4-(3-fluoro- |
| | 4-methoxyphenyl)pyrimidin-2- |
| | ylamino)benzenesulfonamide acetate (Form V) |
| 21. | N-(3-dimethylamino)propyl)-4-(4-(3-fluoro-4- |
| | methoxyphenyl)pyrimidin-2- |
| | ylamino)benzenesulfonamide acetate (Form VI) |
| 22. | N-(3-dimethylamino)propyl)-4-(4-(3-fluoro- |
| | 4-methoxyphenyl)pyrimidin-2- |
| | ylamino)benzenesulfonamide D-glucoronate (Form I) |
| 23. | N-(3-dimethylamino)propyl)-4-(4-(3-fluoromethyl- |
| | 4-methoxyphenyl)pyrimidin-2- |
| | ylamino)benzenesulfonamide D-glucoronate (Form II) |
| 24. | N-(3-dimethylamino)propyl)-4-(4-(3-fluoro- |
| | 4-methoxyphenyl)pyrimidin-2- |
| | ylamino)benzenesulfonamide D-glucoronate (Form III) |
| 25. | N-(3-dimethylamino)propyl)-4-(4-(3-fluoro- |
| | 4-methoxyphenyl)pyrimidin-2- |
| | ylamino)benzenesulfonamide D-glucoronate (Form IV) |

The presence of certain substituents in the compounds of formula I, II, or III may enable salts of the compounds to be formed. Suitable salts include pharmaceutically acceptable salts, for example acid addition salts derived from inorganic or organic acids, and salts derived from inorganic and organic bases. The phrase “pharmaceutically acceptable salt,” as used herein, is a salt formed from an acid and a basic nitrogen group of a pharmaceutically active agent. Illustrative salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts.

The phrase “pharmaceutically acceptable salt” also refers to a salt prepared from a pharmaceutically active agent having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N,-dimethyl-N-(2-hydroxyethyl)amine, or tri-(2-hydroxyethyl)amine; N-methyl-D-glucamine; and amino acids such as arginine, lysine, and the like.

Acid addition salts include hydrochlorides, hydrobromides, hydroiodides, alkylsulphonates, e.g. methanesulphonates, ethanesulphonates, or isethionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, D-, L-tartrates, D-, L-glutamates, D-, L-glucoronates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

According to one embodiment, useful salts of compounds according to the invention include pharmaceutically acceptable salts, especially acid addition pharmaceutically acceptable salts.

In another embodiment, the present invention provides methods for making one or more crystalline forms and polymorphs of a compound of formula I, II, or III as defined above. The term “reacting” includes, but is not limited to, adding, stirring, heating, heating to reflux, dissolving, titurating, and any combination thereof. One skilled in the art would appreciate the meaning of reacting given the reaction components and given the examples provided herein. The methods include a step of isolating the crystalline form or polymorph of compounds of formula I, II or III.

According to one embodiment, the invention provides a method for preparing and manufacturing a crystalline form or polymorph of a compound of formula I in the form of corresponding pharmaceutically acceptable salts, comprising the steps of: dissolving an amount of the compound of formula I and a compound selected from the group consisting of acids, bases and combinations thereof, as a mixture or slurry; and precipitating one crystalline form or polymorph of the compound of formula I, II or III in the form of the pharmaceutically acceptable salt from the mixture or slurry. Precipitating refers to crystallization of the crystalline form or polymorph from the mixture or slurry, from the addition of a different solvent or solvents to the mixture or slurry, from concentration of mixture or slurry, or from cooling of the mixture or slurry.

According to a separate embodiment, the invention provides a method for preparing and manufacturing a crystalline polymorph of a compound of formula I in the form of corresponding pharmaceutically acceptable salts, comprising the step of: recrystallizing one crystalline form or polymorph of the compound of formula I as the pharmaceutically acceptable salt, from one or more solvents, to precipitate a different crystalline polymorph of the compound of formula I, II or III as the pharmaceutically acceptable salt. The one or more solvents may be reduced in volume or diluted with water or solvent, as compared to the solvent used to prepare the polymorph before recrystallizing.

According to a separate embodiment, the invention provides a method for preparing and manufacturing a crystalline polymorph of a compound of formula I in the form of corresponding pharmaceutically acceptable salts, comprising the steps of: dissolving an amount of the compound of formula I and a compound selected from the group consisting of acids, bases and combinations thereof, as a mixture or slurry; precipitating one crystalline form or polymorph of the compound of formula I, II or III in the form of the pharmaceutically acceptable salt from the mixture or slurry; and recrystallizing the precipitated crystalline form or polymorph of the compound of formula I as the pharmaceutically acceptable salt, from the one or more solvents or from one or more different solvents, to precipitate a different crystalline polymorph of the compound of formula I as the pharmaceutically acceptable salt.

The amounts of the compound of formula I, II or III to the compounds selected from the group consisting of acids, bases and combinations thereof may be an equivalent amount by weight, an equivalent amount based on moles of the reactants or an excess amount of the compound of formula I, based on equivalent weights (including weight ratios) or an excess amount of the compound, based on equivalent molar weight.

Compounds combined, mixed or slurried with the compounds of formula I are salt-forming compounds selected from acids, bases and combinations thereof. According to one embodiment, the compounds mixed with free base compounds of formula I, II or III are selected from acids. Suitable acids include organic acids and inorganic acids. Suitable organic acids include, but are not limited to for example, succinic acid, oxalic acid, acetic acid, D-, L-glucoronic acid, citric acid, malic acid, maleic acid, D-, L-glutamic acid, D-, L-tartaric acid and like organic acids.

According to one exemplary embodiment, equivalent molar amounts of succinic acid and the compound of N-(3-(dimethylamino)propyl)-4-(4-(3-fluoro-4-methoxyphenyl)pyrimidin-2-ylamino)benzenesulfonamide are combined to provide one crystalline form of the compound as a mono-succinate salt of formula:

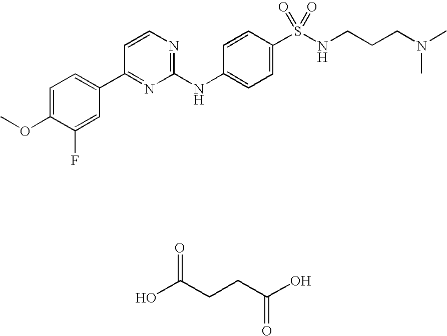

According to a separate exemplary embodiment, half a molar equivalent of an amount of succinic acid is combined with one molar equivalent of the compound N-(3-(dimethylamino)propyl)-4-(4-(3-fluoro-4-methoxyphenyl)pyrimidin-2-ylamino)benzenesulfonamide to provide one crystalline polymorph of the compound as a hemi-succinate of formula:

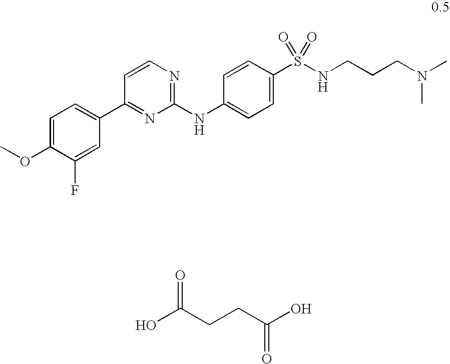

According to another exemplary embodiment, equivalent molar amounts of D-glucoronic acid and the compound of N-(3-(dimethylamino)propyl)-4-(4-(3-fluoro-4-methoxyphenyl)pyrimidin-2-ylamino)benzenesulfonamide are combined to provide one crystalline form of the compound as a mono-D-glucoronate salt of formula:

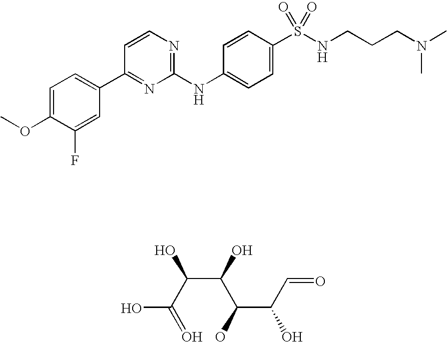

According to yet another exemplary embodiment, equivalent molar amounts of acetic acid and the compound of N-(3-(dimethylamino)propyl)-4-(4-(3-fluoro-4-methoxyphenyl)pyrimidin-2-ylamino)benzenesulfonamide are combined to provide one crystalline form of the compound as a mono-acetate salt of formula:

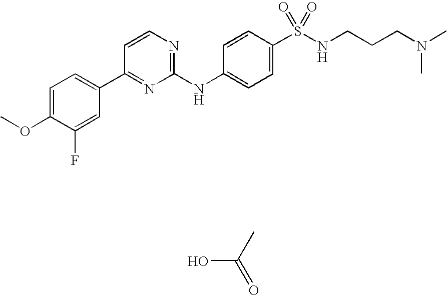

Suitable solvents include water, mixtures of water and conventional organic solvents. Suitable organic solvents, include, but are not limited to for example, acetone, ethanol, methanol, ethyl ether, ethyl acetate, tetrahydrofuran (THF), dimethoxyothane, 1,3-dioxane, furan, ethylene glycol dimethyl ether, anisole, 1-propanol, 2-propanol, 2-methoxyethanol, ethylene glycol, 1-butanol, 2-butanol, diethylene, glycol, monoethyl ether, cyclohexanol, benzyl alcohol, phenol, glycerol, poly(ethylene glycol) (PEG) and related solvents. The volume of water:solvent in the crystallizing solvent ranges from 1:100 to 1000:1, including 1:10 to 100:1, and including 1:5 to 10:1.

According to one embodiment, one polymorph of the compound of formula I, II or III as a pharmaceutically salt is prepared from an organic acid and the compound of formula I, II or III in a certain solvent mixture (e.g., acetone:water). The one crystalline polymorph precipitates by crystallizing out from the mixture of reactants and the one or more solvents. Recrystallizing the precipitated one crystalline polymorph of the compound of formula I, II or III as the pharmaceutically acceptable salt of the organic acid, from a different one or more solvents (e.g., ethanol:water, isopropanol) or from diluting the one or more solvents with water/solvents or from reducing the volume of the one or more solvents, converts the one crystalline polymorph to a different crystalline polymorph of the compound of formula I as the pharmaceutically acceptable organic acid salt, as characterized by polycrystalline XRD.

According to a separate embodiment, the invention provides a method for converting one crystalline polymorph of the compound of formula I, II or III in the form of a pharmaceutically acceptable salt to a different crystalline polymorph of the compound of formula I, II or III as the pharmaceutically acceptable salt, comprising the steps of: heating an amount of one crystalline polymorph of the compound of formula I as the pharmaceutically acceptable salt to a temperature that converts it to a different crystalline polymorph of the compound of formula I, II or III as the pharmaceutically acceptable salt. The temperature needed to convert the one crystalline polymorph to a different crystalline polymorph depends on the thermal stability of the new polymorph relative to the one polymorph. Suitable temperatures for converting one crystalline polymorph to a different crystalline polymorph range from 40° C. to 250° C., including temperatures below the decomposition temperatures of particular crystalline polymorphs.

According to one embodiment, heating one crystalline polymorph of the compound of formula I, II or III in the form of a pharmaceutically acceptable salts converts the one polymorph to successively different crystalline polymorphs of the compound as the pharmaceutically acceptable salt, as each specific conversion range of higher temperatures is achieved. Different crystalline polymorphs are confirmed by analytical methods, including XRD patterns and data, DSC and TGA.

According to a separate embodiment, one crystalline polymorph of a compound of formula I, II or III as a pharmaceutically acceptable salt is converted to a different crystalline polymorph of the compound of formula I, II or III as the pharmaceutically acceptable salt by using a combination of heating and one or more solvents. Converting includes, but is not limited to for example, recrystallizing one crystalline polymorph in one or more solvents to precipitate a different crystalline polymorph, heating one crystalline polymorph to a temperature that converts it to a different crystalline polymorph and recrystallizing one crystalline polymorph in one or more solvents with heating to precipitate a different crystalline polymorph.

Typically, water solubilities of the compounds of formula I, II or III, in the form of a free base, range from low to no water solubility. In an exemplary embodiment, the solubility of the free base form of compound 3 in THF, DMSO and DMF was high but the solubility in group 3 solvents, including water for example, was low. For example, acetone is needed to dissolve the free base at 50° C. Solubility of the free base 3 in water was not detectable by a thermogravimeteric method (N). HCl, phosphoric acid, tartaric acid, acetic acid, D-, L-glucoronic acid and succinic acid were used to prepare pharmaceutically acceptable salts of compound 3. One crystalline form of compound 3 as a mono-succinate salt and five crystalline polymorphs of compound 3 as a hemi-succinate salt are provided in the Examples.

Typically, the crystalline forms and polymorphs thereof can be prepared by dissolving a compound of formula I, II or III, such as compound 3, in a suitable solvent with equivalent weights, equivalent molar weights and weight ratios of salt forming compounds selected from: acids, organic acids, bases, organic bases to the compound of formula I ranging from 1:0.1 to 1:5, based on weight or molar weight, including from 1:0.1 to 1:0.76; from 1:0.1 to 1:0.5; from 1:1 to 1:5 and from 1:1 to 1:>5.

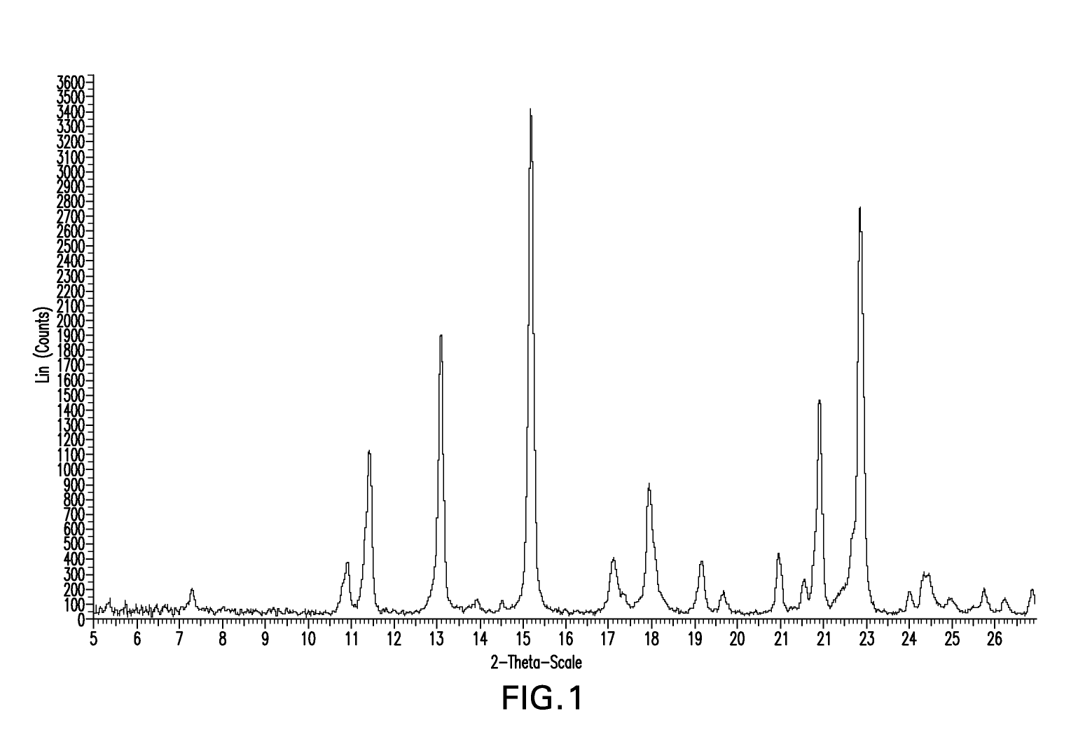

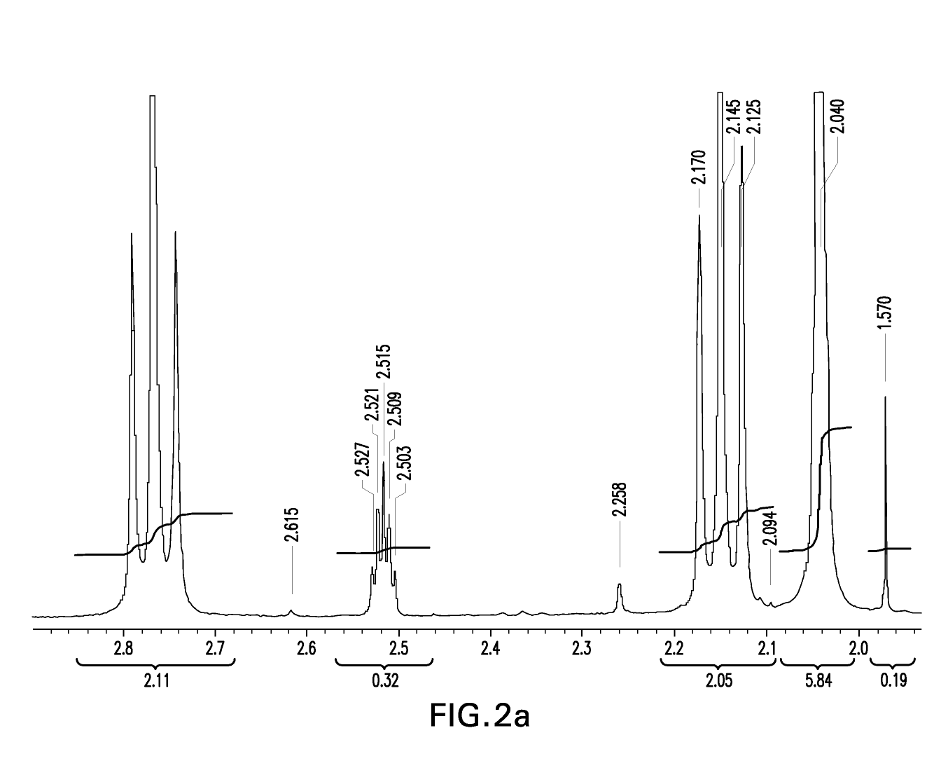

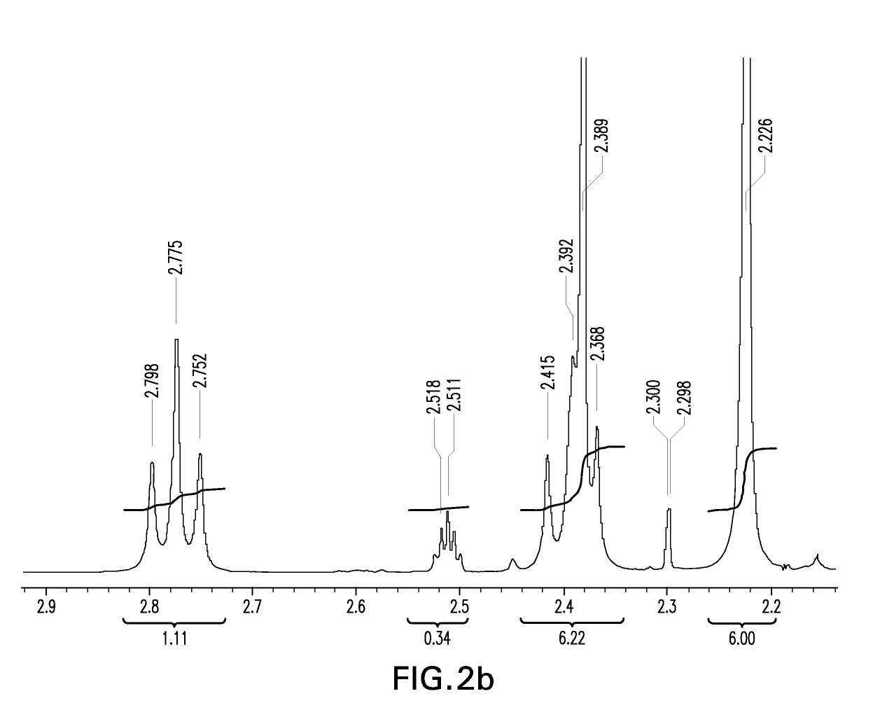

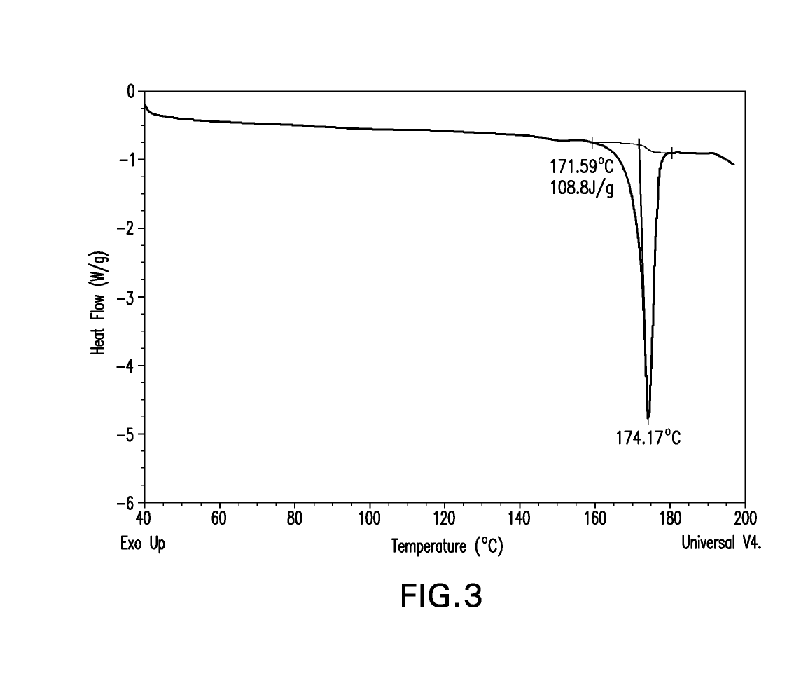

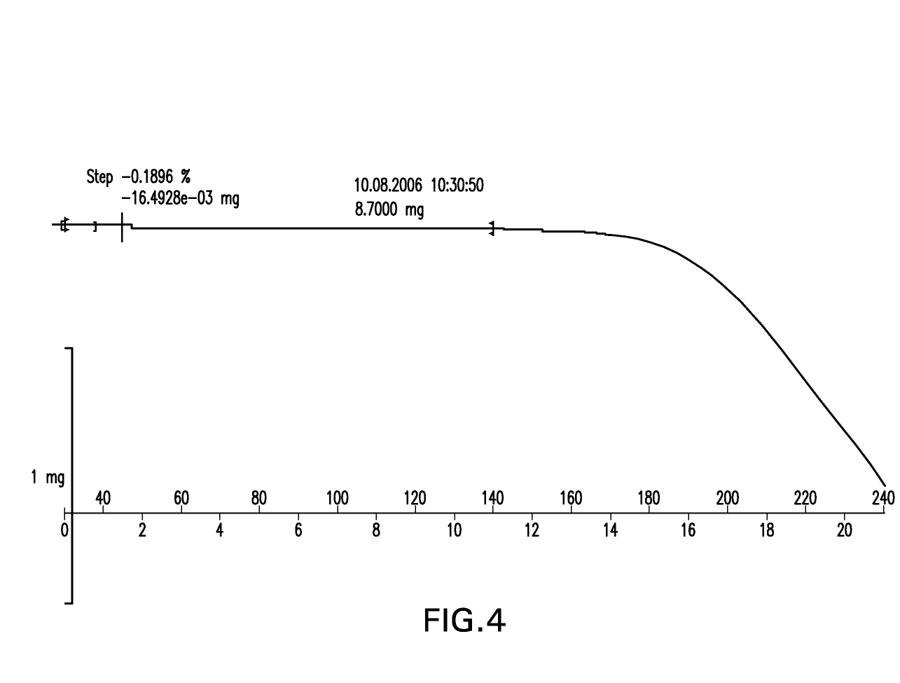

In an exemplary embodiment, mixing compound 3 and succinic acid, in equivalent molar weight ratios of succinic acid to compound 3 ranging from 1:1 to 1:5 produces one crystalline form of compound 3 as a mono-succinate salt. One crystalline form of compound 3 was isolated and characterized, N-(3-(dimethylamino)propyl)-4-(4-(3-fluoro-4-methoxyphenyl)pyrimidin-2-ylamino)-benzenesulfonamide as a mono-succinate salt. The mono-succinate salt is identified by one or more solid-state analytical methods. For example, the mono-succinate salt is identified by characteristic powder X-ray diffraction (XRD) pattern, as shown in FIG. 1. Polycrystalline XRD data consistent with the mono-succinate salt are provided in Table 1 below. One skilled in the art would understand that the relative intensities of the polycrystalline XRD peaks can vary, depending upon the sample preparation technique, the sample mounting procedure and the particular instrument employed. Moreover, instrument variation and other factors can affect the 2-theta values. Therefore, the polycrystalline XRD peak assignments can vary by plus or minus about 0.2°. The NMR spectrum of the free base of compound 3 is shown in FIG. 2a. The NMR spectrum of N-(3-(dimethylamino)propyl)-4-(4-(3-fluoro-4-methoxyphenyl)pyrimidin-2-ylamino)-benzenesulfonamide as a mono-succinate salt is shown in FIG. 2b. The ratio of the free base compound 3 to succinic acid is 1:1 from the analysis of the NMR data. N-(3-(dimethylamino)propyl)-4-(4-(3-fluoro-4-methoxyphenyl)pyrimidin-2-ylamino)-benzenesulfonamide, as a mono-succinate salt, is also identified by its characteristic differential scanning (DSC) trace, as shown in FIG. 3. Melting point is observed at 171.6° C., associated with an endotherm maxima at 174.2° C. N-(3-(dimethylamino)propyl)-4-(4-(3-fluoro-4-methoxyphenyl)pyrimidin-2-ylamino)benzenesulfonamide, as a mono-succinate salt, is also identified by its characteristic thermogravimmetric analysis (TGA) thermogram, as shown in FIG. 4. A weight loss consistent with succinic acid is observed from the TGA themogram.

TABLE 1
X-RAY PEAK POSITIONS OF MONO-SUCCINATE OF N-(3-
(DIMETHYLAMINO)PROPYL)-4-(4-(3-FLUORO-
4-METHOXYPHENYL)PYRIMIDIN-
2-YLAMINO)BENZENESULFONAMIDE

| | d value, | Intensity, | |
|---|---|---|---|
| Angle, 2-Theta ° | Angstrom | Count | Intensity % |
| 7.3 | 12.2 | 182.0 | 5.3 |
| 10.9 | 8.1 | 370.0 | 10.8 |
| 11.4 | 7.7 | 1122.0 | 32.7 |
| 13.1 | 6.8 | 1902.0 | 55.5 |
| 15.2 | 5.8 | 3425.0 | 100.0 |
| 17.1 | 5.2 | 399.0 | 11.6 |
| 17.3 | 5.1 | 166.0 | 4.8 |
| 17.9 | 4.9 | 899.0 | 26.2 |
| 19.2 | 4.6 | 379.0 | 11.1 |
| 19.7 | 4.5 | 173.0 | 5.1 |
| 21.0 | 4.2 | 422.0 | 12.3 |
| 21.6 | 4.1 | 253.0 | 7.4 |
| 21.9 | 4.1 | 1461.0 | 42.7 |
| 22.7 | 3.9 | 569.0 | 16.6 |
| 22.9 | 3.9 | 2765.0 | 80.7 |
| 24.0 | 3.7 | 172.0 | 5.0 |
| 24.4 | 3.7 | 299.0 | 8.7 |
| 24.5 | 3.6 | 289.0 | 8.4 |
| 25.8 | 3.5 | 192.0 | 5.6 |
| 26.9 | 3.3 | 186.0 | 5.4 |

Alternative methods of heating a mixture of free base 3 and succinic acid in water or by heating a mixture of free base 3 and succinic acid in an ethanol:water (5:3 by volume) solvent mixture at 50-55° C. or changing the amount of succinic acid to the amount of freebase 3 (by weight, weight ratio or equivalent molar weights) resulted in the same crystalline form of N-(3-(dimethylamino)propyl)-4-(4-(3-fluoro-4-methoxyphenyl)pyrimidin-2-ylamino)-benzenesulfonamide as a mono-succinate, also identified by its powder X-ray diffraction (XRD) pattern and data, as compared to the polycrystalline XRD data in Table I.

In a separate exemplary embodiment, an equivalent weight ratio of succinic acid to compound 3 of 1:0.76 to 1:0.1 produces one crystalline polymorph of compound 3 as a hemi-succinate salt. The solvent system and temperature appears to play a role in the particular crystalline polymorph formed. One polymorph (Form I) of the hemi-succinate salt of 3 is formed using a acetone:water mixture. Reducing the volume of the acetone:water solvent, as compared to the volume that provides the Form I polymorph, and heating the reactants mixture to 60° C. produces a different crystalline polymorph of the hemi-succinate of 3 (Form II).

The Form II crystalline polymorph of compound 3 as a hemi-succinate salt is used to prepare different crystalline polymorphs of the hemi-succinate salt of compound 3. Heating the Form II polymorph to 70° C. and cooling to room temperature converts the Form II polymorph of the hemi-succinate salt of 3 to a different crystalline polymorph of the hemi-succinate salt of 3, the Form III polymorph. The Form III polymorph was characterized by XRD and other analytical methods. Heating the Form II polymorph to 125° C. and cooling to room temperature converts the Form II polymorph of the hemi-succinate salt of 3 to another different crystalline polymorph of the hemi-succinate salt of 3, the Form IV polymorph. The Form IV polymorph was characterized by XRD and other analytical methods. Heating the Form II polymorph to 140° C. and cooling to room temperature converts the Form II polymorph of the hemi-succinate salt of 3 to yet another different crystalline polymorph of the hemi-succinate salt of 3, the Form V polymorph. The Form V polymorph was characterized by XRD and other analytical methods.

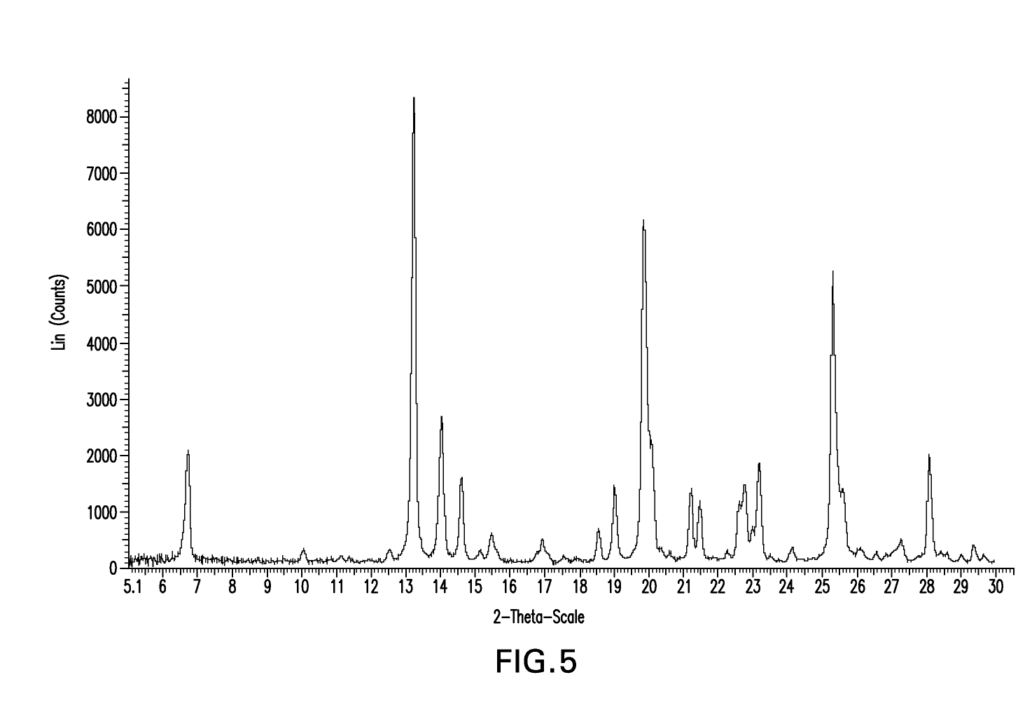

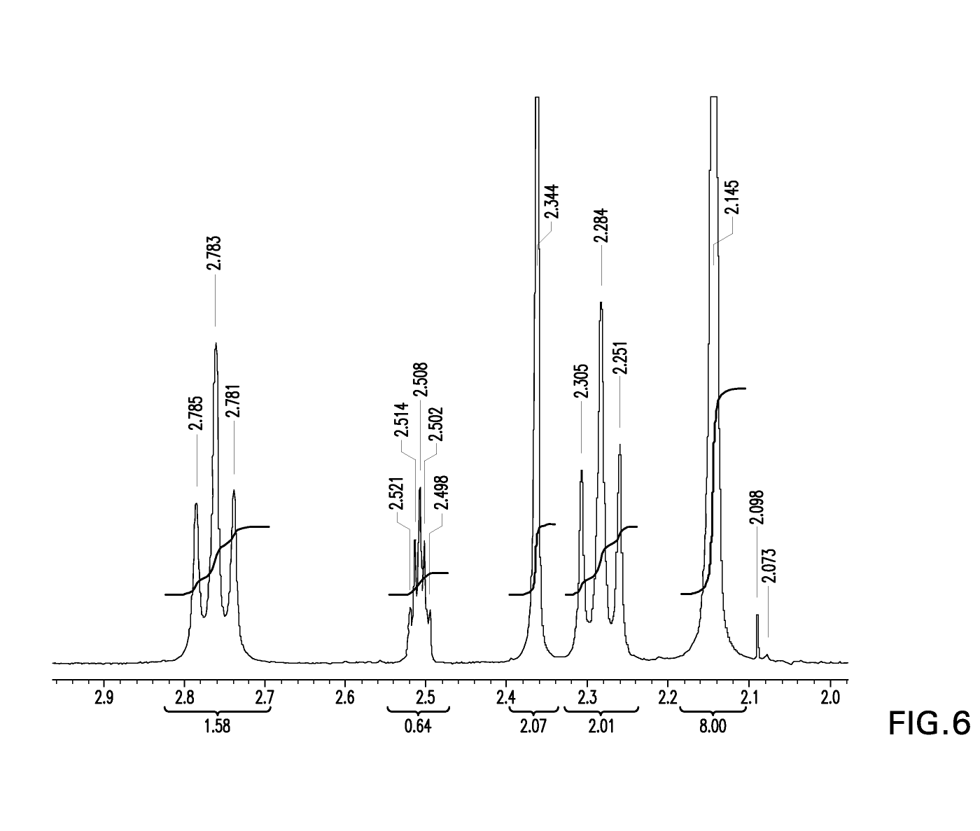

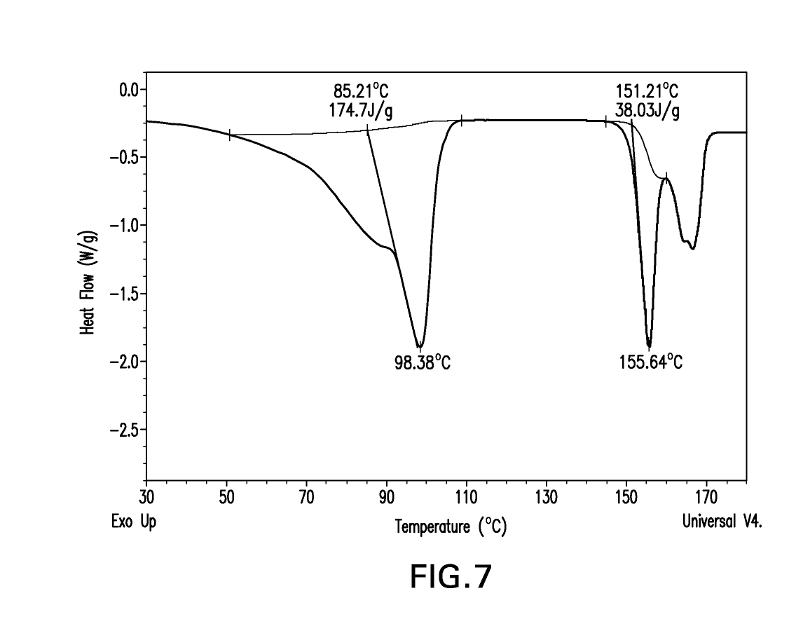

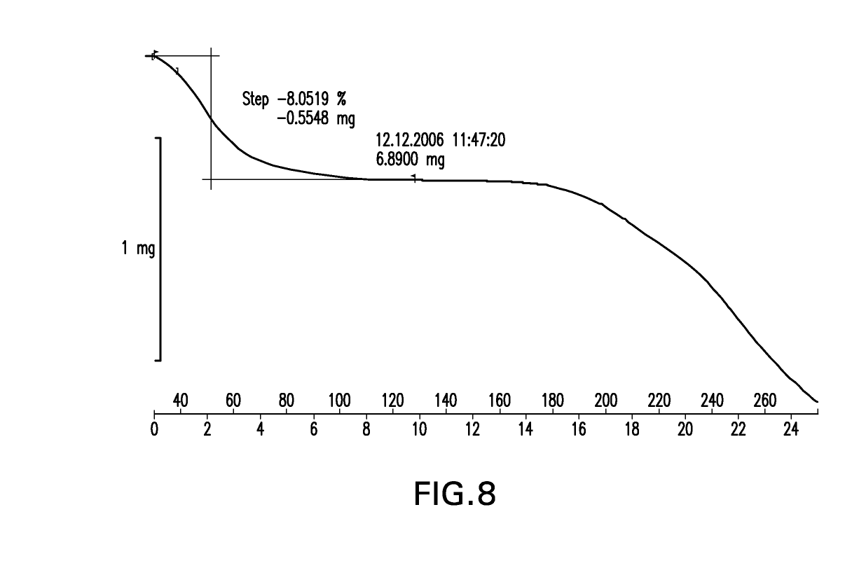

Five crystalline forms or polymorphs of compound 3 were isolated and characterized as hemi-succinate salts. One crystalline polymorph, N-(3-(dimethylamino)propyl)-4-(4-(3-fluoro-4-methoxyphenyl)pyrimidin-2-ylamino)-benzenesulfonamide as a hemi-succinate, is referred to herein as Form I. The Form I polymorph is identified by one or more solid-state analytical methods. The Form I polymorph is identified by its characteristic polycrystalline XRD pattern, as shown in FIG. 5. Polycrystalline XRD data consistent with the Form I polymorph are provided in Table 2 below. The NMR spectrum of N-(3-(dimethylamino)propyl)-4-(4-(3-fluoro-4-methoxyphenyl)pyrimidin-2-ylamino)-benzenesulfonamide as a hemi-succinate salt is shown in FIG. 6. The ratio of succinate to compound 3 was determined to be 2:1 based on NMR analysis. N-(3-(dimethylamino)propyl)-4-(4-(3-fluoro-4-methoxyphenyl)pyrimidin-2-ylamino)-benzenesulfonamide, as a hemi-succinate salt, is also identified by its characteristic differential scanning (DSC) trace, as shown in FIG. 7. The melting point of the hemi-succinate is 125° C. from the broad endotherm indicated by DSC analysis. One broad endotherm having maxima at 98.4° C. is also observed and another large endotherm having maxima at 155.6° C. is also observed. Yet another large endotherm having maxima at 165° C. is observed. N-(3-(dimethylamino)propyl)-4-(4-(3-fluoro-4-methoxyphenyl)pyrimidin-2-ylamino)-benzenesulfonamide as a hemi-succinate is also identified by its characteristic TGA thermogram, as shown in FIG. 8. A weight loss consistent with succinic acid was observed in the TGA themogram. The Form I hemi-succinate salt of compound 3 was prepared by mixing 2 molar equivalents of the free base 3 in an acetone:water solvent (5:2 by volume) and one molar equivalent of succinic acid and heating the mixture to 50-55° C. until the reactants of the mixture dissolved. Adding an additional amount of acetone to the mixture followed by cooling the mixture to room temperature, allowed crystallization of the Form I hemi-succinate salt, precipitating out of the solvent mixture in pure form. The Form I polymorphic salt was dried under vacuum at 50° C. and characterized by XRD and other analytical methods detailed herein.

TABLE 2
X-RAY PEAK POSITIONS OF FORM I HEMI-SUCCINATE OF
N-(3-(DIMETHYLAMINO)PROPYL)-4-(4-(3-FLUORO-4-
METHOXYPHENYL)-PYRIMIDIN-2-
YLAMINO)BENZENESULFONAMIDE

| Angle, 2-Theta ° | D value, Angstrom | Intensity, Count | Intensity % |
|---|---|---|---|
| 6.7 | 13.2 | 2092.0 | 25.1 |
| 13.2 | 6.7 | 8346.0 | 100.0 |
| 14.0 | 6.3 | 2683.0 | 32.1 |
| 14.6 | 6.1 | 1596.0 | 19.1 |
| 15.5 | 5.7 | 592.0 | 7.1 |
| 16.9 | 5.2 | 521.0 | 6.2 |
| 18.6 | 4.8 | 700.0 | 8.4 |
| 19.0 | 4.7 | 1423.0 | 17.1 |
| 19.9 | 4.5 | 6200.0 | 74.3 |
| 20.1 | 4.4 | 2322.0 | 27.8 |
| 21.2 | 4.2 | 1403.0 | 16.8 |
| 21.5 | 4.1 | 1190.0 | 14.3 |
| 22.6 | 3.9 | 1189.0 | 14.2 |
| 22.8 | 3.9 | 1481.0 | 17.7 |
| 23.2 | 3.8 | 1860.0 | 22.3 |
| 25.3 | 3.5 | 5266.0 | 63.1 |
| 25.6 | 3.5 | 1403.0 | 16.8 |
| 27.3 | 3.3 | 513.0 | 6.2 |
| 28.1 | 3.2 | 2000.0 | 24.0 |

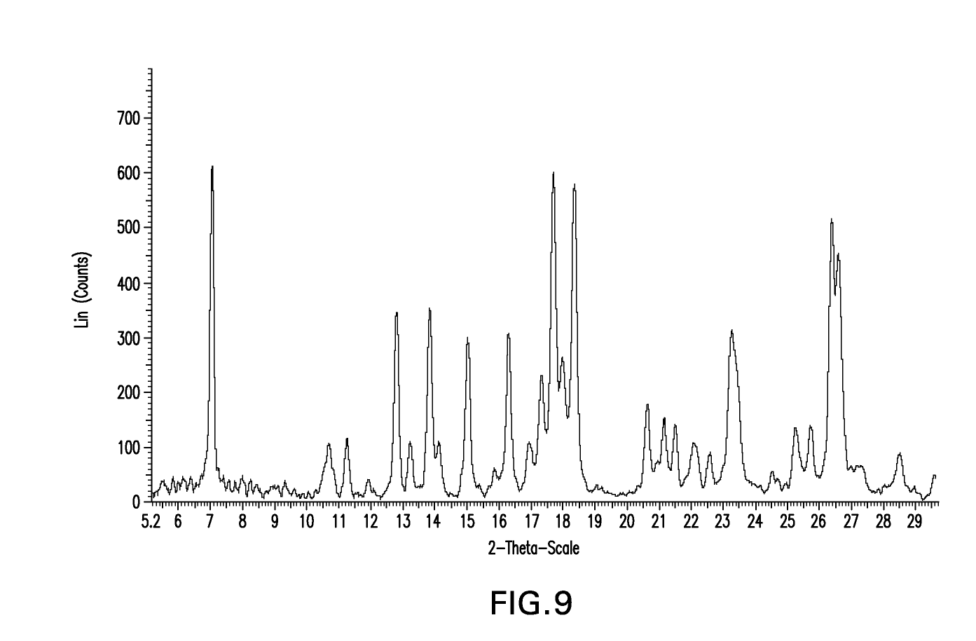

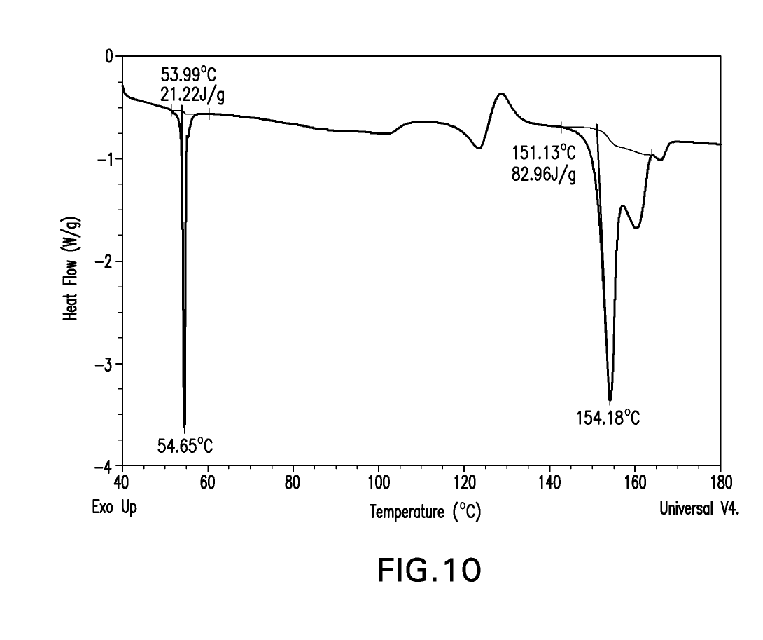

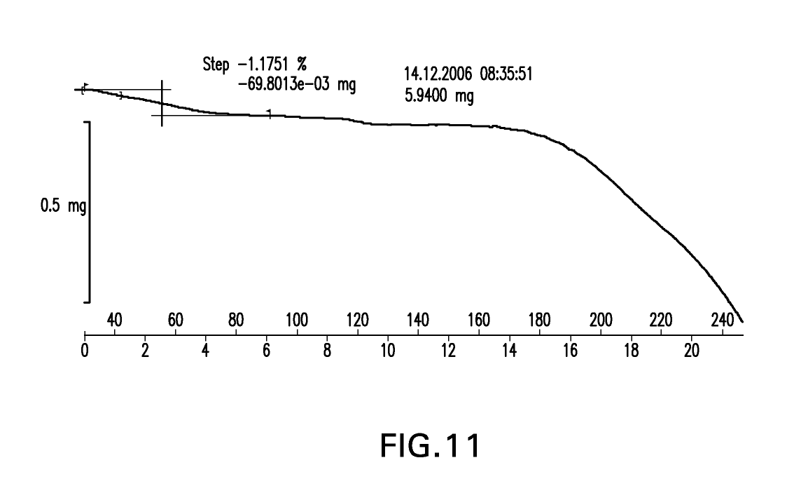

The present invention provides another different crystalline polymorph of N-(3-dimethylamino)propyl)-4-(4-(3-fluoro-4-methoxyphenyl)pyrimidin-2-ylamino)benzenesulfonamide as a hemi-succinate, referred to herein as the Form II polymorph. The Form II polymorph is identified by one or more solid-state analytical methods. The Form II polymorph is identified by characteristic polycrystalline XRD pattern, as shown in FIG. 9. Polycrystalline XRD data consistent with the Form II polymorph are provided in Table 3 below. The Form II polymorph was prepared in a similar manner to Form I, but by reducing the volume of the acetone:water solvent and heating the mixture to 60° C., which resulted in a different crystalline polymorph, Form II. The Form II polymorph precipitated out of the relatively concentrated acetone:water mixture on cooling to provide pure Form II as the hemi-succinate salt. N-(3-(dimethylamino)propyl)-4-(4-(3-fluoro-4-methoxyphenyl)pyrimidin-2-ylamino)-benzenesulfonamide as a hemi-succinate is also identified by its characteristic DSC trace, as shown in FIG. 10. The melting point of the hemi-succinate is 125° C. from the broad endotherm indicated by DSC analysis. One endotherm of maxima at 54.7° C. and a broad endothem with maxima at 125° C. is observed. Another endotherm having maxima at 154.2° C. is also observed. N-(3-(dimethylamino)propyl)-4-(4-(3-fluoro-4-methoxyphenyl)pyrimidin-2-ylamino)benzenesulfonamide as a hemi-succinate is also identified by its characteristic TGA thermogram, as shown in FIG. 11. A weight loss consistent with succinic acid is observed in the TGA themogram. Based on DSC and TGA data, the Form II polymorph was used to prepare different crystalline polymorphs of compound 3 as the hemi-succinate salt.

TABLE 3
X-RAY PEAK POSITIONS OF FORM II HEMI-SUCCINATE OF
N-(3-(DIMETHYLAMINO)PROPYL)-4-(4-(3-FLUORO-4-
METHOXYPHENYL)-PYRIMIDIN-2-
YLAMINO)BENZENESULFONAMIDE

| Angle, 2-Theta ° | d value, Angstrom | Intensity, Count | Intensity % |
|---|---|---|---|
| 5.1 | 17.3 | 32.2 | 5.3 |
| 5.8 | 15.2 | 44.3 | 7.2 |
| 7.0 | 12.6 | 611.0 | 100.0 |
| 8.0 | 11.1 | 42.4 | 6.9 |
| 8.2 | 10.8 | 37.9 | 6.2 |
| 10.7 | 8.3 | 104.0 | 17.1 |
| 11.2 | 7.9 | 114.0 | 18.6 |
| 12.8 | 6.9 | 345.0 | 56.4 |
| 13.2 | 6.7 | 108.0 | 17.7 |
| 13.8 | 6.4 | 353.0 | 57.7 |
| 14.1 | 6.3 | 109.0 | 17.9 |
| 15.0 | 5.9 | 298.0 | 48.7 |
| 16.3 | 5.4 | 306.0 | 50.1 |
| 17.0 | 5.2 | 108.0 | 17.6 |
| 17.3 | 5.1 | 230.0 | 37.6 |
| 17.7 | 5.0 | 602.0 | 98.4 |
| 18.0 | 4.9 | 263.0 | 43.1 |
| 18.4 | 4.8 | 579.0 | 94.7 |
| 20.6 | 4.3 | 177.0 | 29.0 |
| 21.2 | 4.2 | 151.0 | 24.7 |
| 21.5 | 4.1 | 140.0 | 22.9 |
| 22.1 | 4.0 | 107.0 | 17.6 |
| 22.6 | 3.9 | 88.4 | 14.5 |
| 23.3 | 3.8 | 313.0 | 51.2 |
| 23.5 | 3.8 | 247.0 | 40.4 |
| 25.3 | 3.5 | 134.0 | 21.9 |
| 25.7 | 3.5 | 137.0 | 22.5 |
| 26.4 | 3.4 | 517.0 | 84.6 |
| 26.6 | 3.3 | 453.0 | 74.1 |
| 28.5 | 3.1 | 86.4 | 14.1 |
| 29.6 | 3.0 | 48.6 | 7.9 |

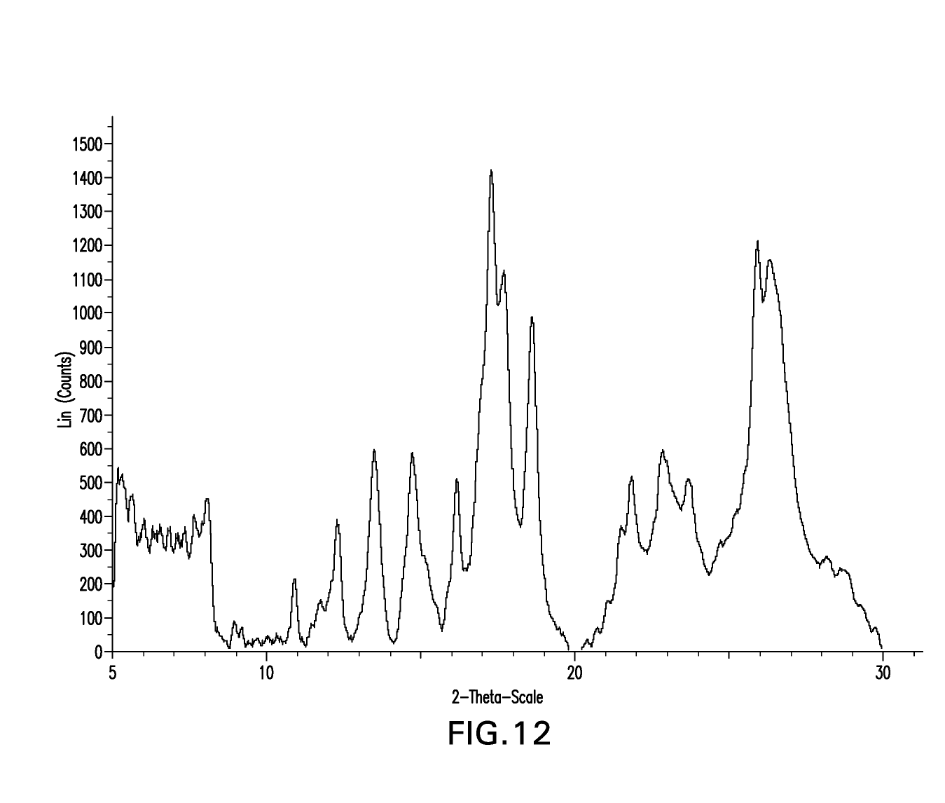

The present invention provides yet another crystalline polymorph of N-(3-dimethylamino)propyl)-4-(4-(3-fluoro-4-methoxyphenyl)pyrimidin-2-ylamino)-benzenesulfonamide as a hemi-succinate, referred to herein as the Form III polymorph. The Form III polymorph is identified by one or more solid-state analytical methods. The Form III polymorph is identified by characteristic polycrystalline XRD pattern, as shown in FIG. 12. Polycrystalline XRD data consistent with the Form III are provided in Table 4 below. Heating a solid-state sample of the Form II polymorph to 70° C. converted the Form II polymorph to a new polymorph, Form III hemi-succinate salt in pure form. The new crystalline polymorph Form III hemi-succinate exhibited a characteric XRD pattern and data.

TABLE 4
X-RAY PEAK POSITIONS OF FORM III HEMI-SUCCINATE
OF N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 10.9 | 8.1 | 210.0 | 14.7 |
| 11.5 | 7.7 | 77.5 | 5.4 |
| 11.7 | 7.5 | 150.0 | 10.5 |
| 12.3 | 7.2 | 387.0 | 27.1 |
| 13.0 | 6.8 | 105.0 | 7.4 |
| 13.5 | 6.6 | 594.0 | 41.6 |
| 14.7 | 6.0 | 587.0 | 41.1 |
| 15.1 | 5.9 | 282.0 | 19.8 |
| 15.5 | 5.7 | 134.0 | 9.4 |
| 15.9 | 5.6 | 189.0 | 13.3 |
| 16.2 | 5.5 | 506.0 | 35.5 |
| 17.0 | 5.2 | 793.0 | 55.6 |
| 17.3 | 5.1 | 1426.0 | 100.0 |
| 17.7 | 5.0 | 1129.0 | 79.2 |
| 18.6 | 4.8 | 988.0 | 69.3 |
| 20.7 | 4.3 | 65.8 | 4.6 |
| 21.1 | 4.2 | 147.0 | 10.3 |
| 21.5 | 4.1 | 369.0 | 25.9 |
| 21.8 | 4.1 | 516.0 | 36.2 |
| 22.9 | 3.9 | 595.0 | 41.7 |
| 23.7 | 3.8 | 508.0 | 35.6 |
| 25.3 | 3.5 | 418.0 | 29.3 |
| 25.9 | 3.4 | 1214.0 | 85.2 |
| 26.3 | 3.4 | 1160.0 | 81.4 |
| 28.9 | 3.1 | 232.0 | 16.3 |
| 5.1 | 17.2 | 538.0 | 37.7 |
| 5.3 | 16.8 | 523.0 | 36.7 |
| 5.6 | 15.9 | 457.0 | 32.0 |
| 6.0 | 14.8 | 390.0 | 27.4 |
| 6.5 | 13.6 | 355.0 | 24.9 |
| 6.8 | 13.0 | 358.0 | 25.1 |
| 7.1 | 12.5 | 338.0 | 23.7 |
| 7.3 | 12.1 | 358.0 | 25.1 |
| 7.6 | 11.6 | 401.0 | 28.2 |
| 8.0 | 11.0 | 448.0 | 31.4 |
| 8.9 | 9.9 | 84.3 | 5.9 |
| 10.9 | 8.1 | 210.0 | 14.7 |
| 11.5 | 7.7 | 77.5 | 5.4 |
| 11.7 | 7.5 | 150.0 | 10.5 |
| 12.3 | 7.2 | 387.0 | 27.1 |
| 13.0 | 6.8 | 105.0 | 7.4 |
| 13.5 | 6.6 | 594.0 | 41.6 |
| 14.7 | 6.0 | 587.0 | 41.1 |
| 15.1 | 5.9 | 282.0 | 19.8 |
| 15.5 | 5.7 | 134.0 | 9.4 |
| 15.9 | 5.6 | 189.0 | 13.3 |
| 16.2 | 5.5 | 506.0 | 35.5 |
| 17.0 | 5.2 | 793.0 | 55.6 |
| 17.3 | 5.1 | 1426.0 | 100.0 |
| 17.7 | 5.0 | 1129.0 | 79.2 |
| 18.6 | 4.8 | 988.0 | 69.3 |
| 21.1 | 4.2 | 147.0 | 10.3 |
| 21.5 | 4.1 | 369.0 | 25.9 |
| 21.8 | 4.1 | 516.0 | 36.2 |
| 22.9 | 3.9 | 595.0 | 41.7 |
| 23.7 | 3.8 | 508.0 | 35.6 |
| 25.3 | 3.5 | 418.0 | 29.3 |
| 25.9 | 3.4 | 1214.0 | 85.2 |
| 26.3 | 3.4 | 1160.0 | 81.4 |
| 28.9 | 3.1 | 232.0 | 16.3 |

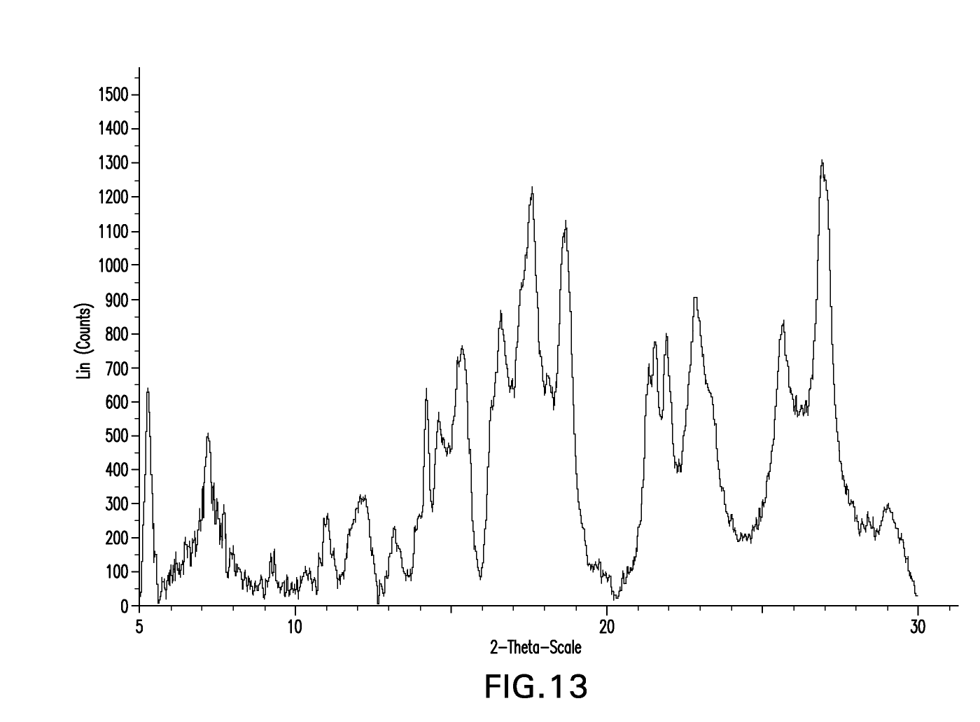

The present invention provides yet another crystalline polymorph of N-(3-(dimethylamino)propyl)-4-(4-(3-fluoro-4-methoxyphenyl)pyrimidin-2-ylamino)benzenesulfonamide as a hemi-succinate, referred to herein as the Form IV polymorph. The Form IV polymorph is identified by one or more solid-state analytical methods. The Form IV polymorph is identified by characteristic polycrystalline XRD pattern, as shown in FIG. 13. Polycrystalline XRD data consistent with Form IV are provided in Table 5 below. Heating a solid-state sample of the Form II polymorph to 125° C. converted the Form II polymorph to a new polymorph, the Form IV polymorph of the hemi-succinate salt in pure form. The new crystalline polymorph Form IV hemi-succinate exhibited a characteristic XRD pattern and data.

TABLE 5
X-RAY PEAK POSITIONS OF FORM IV HEMI-SUCCINATE
OF N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 5.2 | 16.9 | 587.0 | 45.3 |
| 6.5 | 13.6 | 172.0 | 13.3 |
| 7.2 | 12.3 | 496.0 | 38.3 |
| 7.6 | 11.6 | 238.0 | 18.3 |
| 7.9 | 11.1 | 149.0 | 11.5 |
| 9.2 | 9.6 | 129.0 | 10.0 |
| 11.0 | 8.1 | 260.0 | 20.0 |
| 11.8 | 7.5 | 229.0 | 17.6 |
| 12.1 | 7.3 | 316.0 | 24.4 |
| 13.2 | 6.7 | 214.0 | 16.5 |
| 13.9 | 6.3 | 253.0 | 19.5 |
| 14.2 | 6.2 | 573.0 | 44.2 |
| 14.6 | 6.1 | 533.0 | 41.2 |
| 15.4 | 5.8 | 751.0 | 58.0 |
| 16.3 | 5.4 | 564.0 | 43.6 |
| 16.6 | 5.3 | 839.0 | 64.8 |
| 17.3 | 5.1 | 969.0 | 74.8 |
| 17.6 | 5.0 | 1213.0 | 93.6 |
| 18.6 | 4.8 | 1107.0 | 85.4 |
| 20.0 | 4.4 | 83.9 | 6.5 |
| 21.4 | 4.2 | 678.0 | 52.3 |
| 21.6 | 4.1 | 749.0 | 57.8 |
| 21.9 | 4.0 | 761.0 | 58.8 |
| 22.9 | 3.9 | 900.0 | 69.4 |
| 23.3 | 3.8 | 633.0 | 48.9 |
| 25.7 | 3.5 | 825.0 | 63.7 |
| 27.0 | 3.3 | 1295.0 | 100.0 |

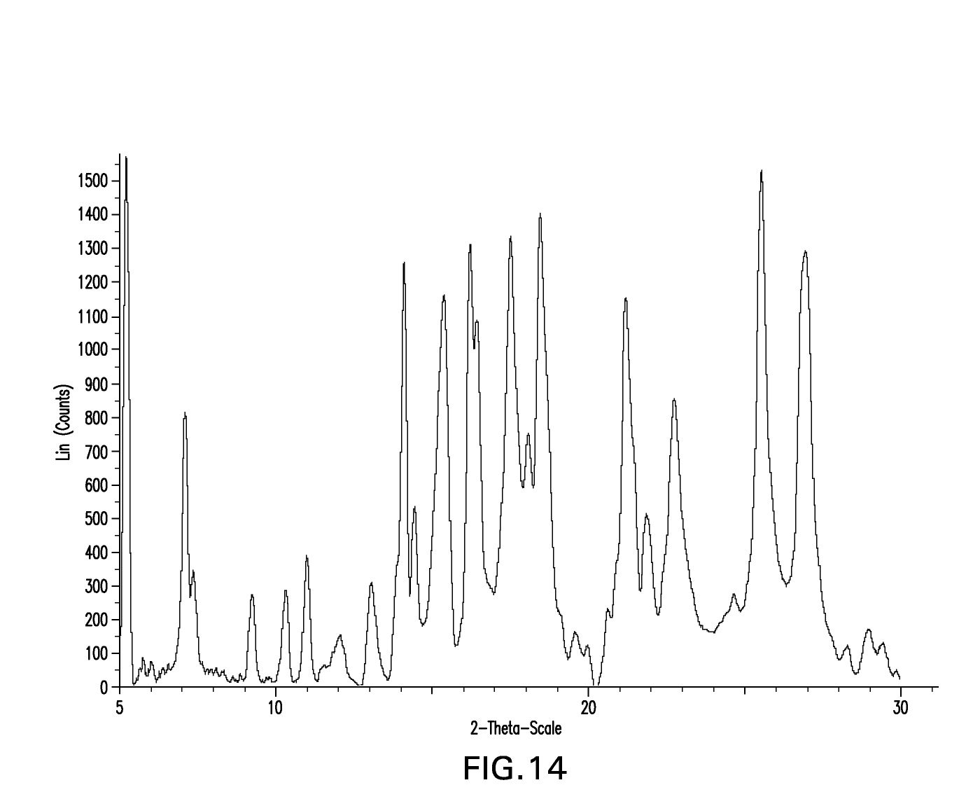

The present invention provides yet another crystalline polymorph of N-(3-(dimethylamino)propyl)-4-(4-(3-fluoro-4-methoxyphenyl)pyrimidin-2-ylamino)-benzenesulfonamide as a hemi-succinate salt, referred to herein as the Form V polymorph. The Form V polymorph is identified by one or more solid-state analytical methods. The Form V polymorph is identified by characteristic powder X-ray diffraction (XRD) pattern, as shown in FIG. 14. Polycrystalline XRD data consistent with the Form V polymorphs are provided in Table 6 below. Heating a solid-state sample of the Form II polymorph to 140° C. converted the Form II polymorph to a new polymorph, Form V of the hemi-succinate salt in pure form. The new crystalline polymorph Form V hemi-succinate exhibited a characteristic XRD pattern and data.

TABLE 6
X-RAY PEAK POSITIONS OF FORM V HEMI-SUCCINATE OF
N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 5.2 | 17.1 | 1577.0 | 100.0 |
| 7.1 | 12.5 | 812.0 | 51.5 |
| 7.3 | 12.1 | 344.0 | 21.8 |
| 9.2 | 9.6 | 271.0 | 17.2 |
| 10.3 | 8.6 | 283.0 | 18.0 |
| 11.0 | 8.1 | 384.0 | 24.4 |
| 13.0 | 6.8 | 308.0 | 19.5 |
| 13.8 | 6.4 | 354.0 | 22.5 |
| 14.1 | 6.3 | 1260.0 | 79.9 |
| 14.4 | 6.1 | 531.0 | 33.7 |
| 15.4 | 5.8 | 1162.0 | 73.7 |
| 16.2 | 5.5 | 1314.0 | 83.3 |
| 16.4 | 5.4 | 1088.0 | 69.0 |
| 17.5 | 5.1 | 1339.0 | 84.9 |
| 18.1 | 4.9 | 753.0 | 47.7 |
| 18.5 | 4.8 | 1407.0 | 89.2 |
| 18.7 | 4.7 | 933.0 | 59.2 |
| 19.6 | 4.5 | 158.0 | 10.0 |
| 20.0 | 4.4 | 119.0 | 7.6 |
| 20.6 | 4.3 | 227.0 | 14.4 |
| 21.2 | 4.2 | 1156.0 | 73.3 |
| 21.9 | 4.1 | 511.0 | 32.4 |
| 22.7 | 3.9 | 854.0 | 54.2 |
| 25.5 | 3.5 | 1535.0 | 97.4 |
| 27.0 | 3.3 | 1294.0 | 82.1 |

One advantage of the mono-acetate salt was its relatively high water solubility, 3.5 mg/mL, as compared to mono-succinate (1.1 mg/mL) salts of compound 3. Examples of preparations of Forms I-VI are provided in the Examples. In general, the Form I polymorph can be prepared by mixing compound 3 (any form, including amorphous) and an equivalent molar amount of acetic acid in an ether solvent (e.g. THF) containing water and heating the mixture to 50° C., precipitating one crystalline polymorph product from the mixture by any of numerous routine methods in the art such as by cooling or evaporating the solvent to induce precipitation. Suitable solvents include but are not limited to for example, water, a mixture of water and an alcohol, water and an ether, water and an ester, water mixture with conventional organic solvents and any suitable organic solvents. The Form II polymorph can be prepared by mixing compound 3 (any form, including amorphous) and an equivalent molar amount of acetic acid in a different solvent (e.g. ethylacetate). Alternatively, the Form II polymorph is also prepared using a smaller volume of ethyl acetate and heating the mixture to 60° C. The Form III polymorph is prepared by mixing compound 3 (any form, including amorphous) and an equivalent amount molar of acetic acid in yet a different solvent (e.g. isopropanol) and heating the mixture to about 60-65° C. The Form II polymorph is converted to a different crystalline polymorph, Form IV polymorph, by recrystallizing the Form II polymorph in acetone, which provides the Form IV polymorph. Water content of the solvent appears to influence the relative amounts of which crystalline polymorph that precipitates, Form II or Form IV polymorph. Higher amounts of water in the solvent tend to favor the Form IV polymorph, while lower amounts of water tend to favor the Form II polymorph. The Form II polymorph is converted to another different crystalline polymorph, Form V polymorph, by recrystallizing the Form II polymorph in water, which provides the Form V polymorph. Water content of the solvent appears to influence the relative amounts of which crystalline polymorph that precipitates, Form II or Form V polymorph. Higher amounts of water in the solvent tend to favor the Form V polymorph, while lower amounts of water tend to favor the Form II polymorph. The Form II polymorph is also used to prepare or is converted to yet another different crystalline polymorph, Form VI polymorph, by recrystallizing the Form II polymorph in a mixture of methanol and ethanol, which provides the Form VI polymorph.

In an exemplary embodiment, a weight ratio, based on molar weight, of acetic acid to a free base such as compound 3 is between 1:1 to 1:5. The solvent system also appears to play a role in converting of one polymorph to other polymorphs.

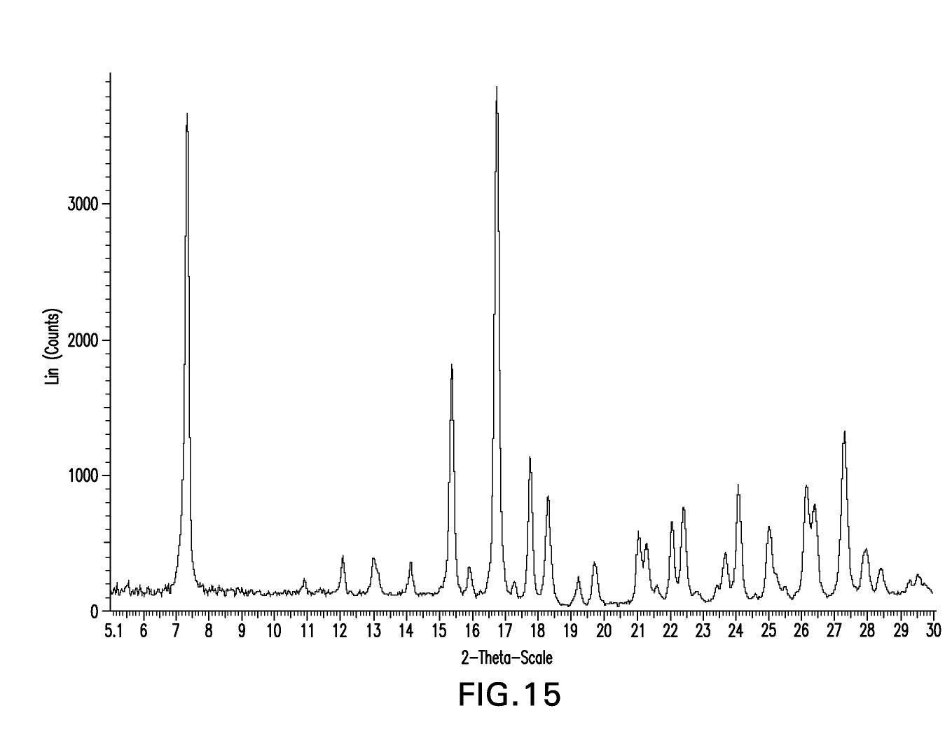

According to one embodiment, six crystalline polymorphs of compound 3 were isolated and characterized as acetate salts. The present invention provides one crystalline polymorph of N-(3-(dimethylamino)propyl)-4-(4-(3-fluoro-4-methoxyphenyl)pyrimidin-2-ylamino)benzenesulfonamide as an acetate salt, referred to herein as Form I. The Form I polymorph is identified by one or more solid-state analytical methods. The Form I polymorph exhibits a characteristic polycrystalline X-ray diffraction (XRD) pattern, as shown in FIG. 15. Polycrystalline XRD data consistent with the Form I polymorph are provided in Table 7 below.

TABLE 7
X-RAY PEAK POSITIONS OF FORM I ACETATE OF N-(3-
(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 7.3 | 12.1 | 36718.0 | 94.8 |
| 10.9 | 8.1 | 2290.0 | 5.9 |
| 12.1 | 7.3 | 3911.0 | 10.1 |
| 13.0 | 6.8 | 3780.0 | 9.8 |
| 14.1 | 6.3 | 3559.0 | 9.2 |
| 15.4 | 5.8 | 18172.0 | 46.9 |
| 15.9 | 5.6 | 3156.0 | 8.2 |
| 16.7 | 5.3 | 38722.0 | 100.0 |
| 17.3 | 5.1 | 2033.0 | 5.3 |
| 17.8 | 5.0 | 11313.0 | 29.2 |
| 18.3 | 4.8 | 8414.0 | 21.7 |
| 19.2 | 4.6 | 2404.0 | 6.2 |
| 19.7 | 4.5 | 3546.0 | 9.2 |
| 21.1 | 4.2 | 5800.0 | 15.0 |
| 21.3 | 4.2 | 4838.0 | 12.5 |
| 22.1 | 4.0 | 6486.0 | 16.7 |
| 22.4 | 4.0 | 7565.0 | 19.5 |
| 23.7 | 3.8 | 4251.0 | 11.0 |
| 24.1 | 3.7 | 9329.0 | 24.1 |
| 25.0 | 3.6 | 6168.0 | 15.9 |
| 26.2 | 3.4 | 9218.0 | 23.8 |
| 26.4 | 3.4 | 7785.0 | 20.1 |
| 27.3 | 3.3 | 13113.0 | 33.9 |
| 28.0 | 3.2 | 4505.0 | 11.6 |
| 28.4 | 3.1 | 3063.0 | 7.9 |
| 29.6 | 3.0 | 2608.0 | 6.7 |

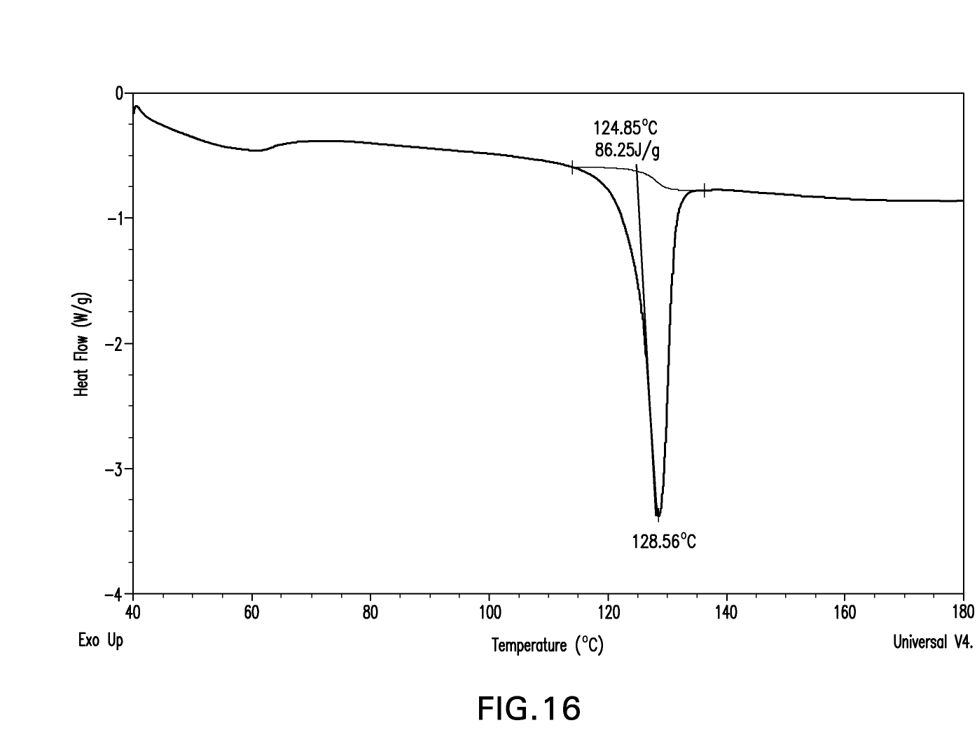

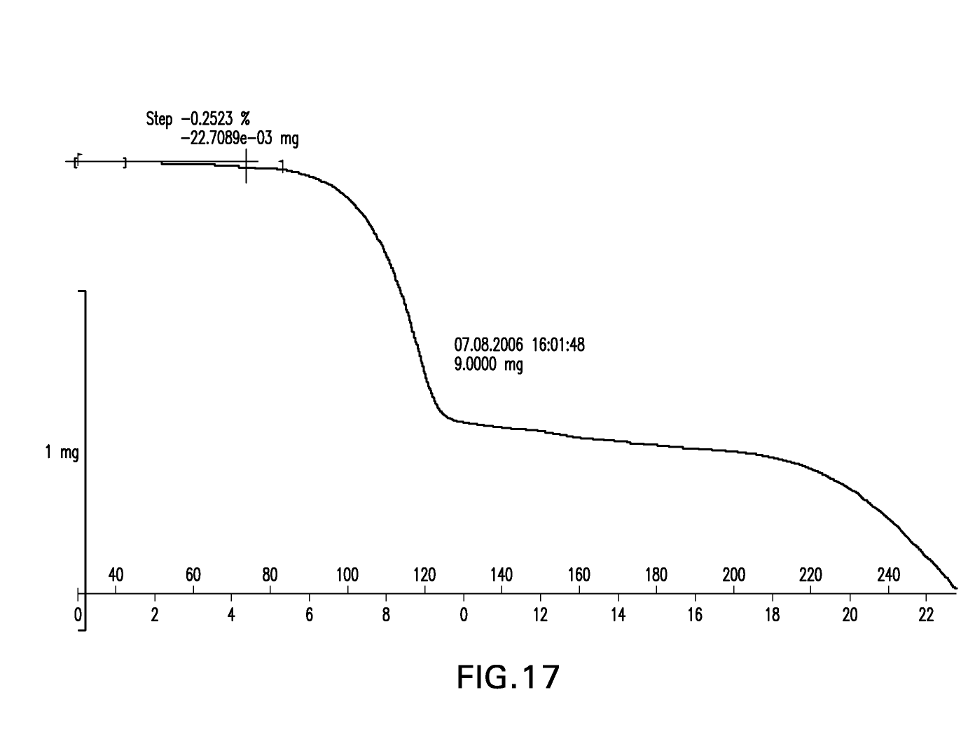

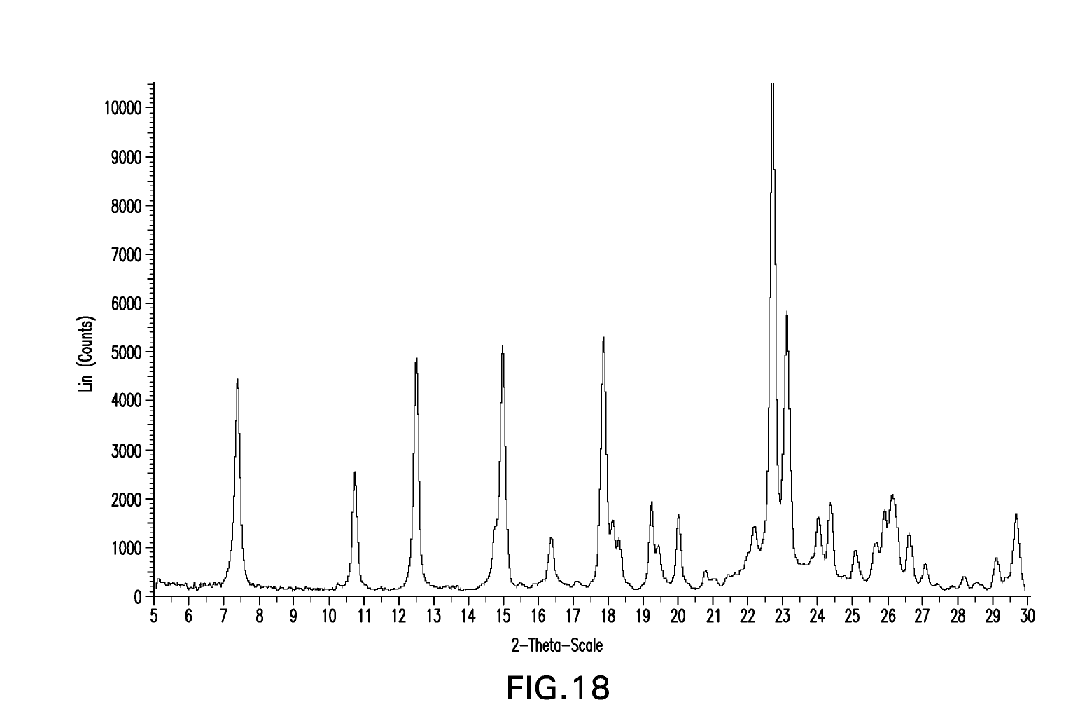

The present invention provides another different crystalline polymorph of N-(3-(dimethylamino)propyl)-4-(4-(3-fluoro-4-methoxyphenyl)pyrimidin-2-ylamino)benzenesulfonamide as an acetate salt, referred to herein as Form II. The Form II polymorph is identified by one or more solid-state analytical methods. N-(3-(dimethylamino)propyl)-4-(4-(3-fluoro-4-methoxyphenyl)pyrimidin-2-ylamino)-benzenesulfonamide as an acetate salt, is also identified by its characteristic differential scanning (DSC) trace, as shown in FIG. 16. A small endotherm having maxima at 60° C. is observed and melting point is observed at 124.9° C., with an endotherm having maxima at 128.6° C. The ratio of free base compound 3 to acetic acid is 1:1. N-(3-(dimethylamino)propyl)-4-(4-(3-fluoro-4-methoxyphenyl)pyrimidin-2-ylamino)benzenesulfonamide as an acetate salt, is also identified by its characteristic thermogravimmetric analysis (TGA) thermmogram, as shown in FIG. 17. A weight loss consistent with acetic acid is observed from the TGA themogram. The Form II polymorph exhibits a characteristic polycrystalline XRD pattern, as shown in FIG. 18. Polycrystalline XRD data consistent with the Form II polymorph are provided in Table 8 below.

TABLE 8
X-RAY PEAK POSITIONS OF FORM II ACETATE OF N-(3-
(DIMETHYLAMINO)PROPYL)-4-(4-(3-FLUORO-4-
METHOXYPHENYL)-PYRIMIDIN-
2-YLAMINO)BENZENESULFONAMIDE

| Angle, 2-Theta ° | d value, Angstrom | Intensity, Count | Intensity % |
|---|---|---|---|
| 7.4 | 12.0 | 4428.0 | 40.4 |
| 10.7 | 8.2 | 2516.0 | 23.0 |
| 12.5 | 7.1 | 4855.0 | 44.3 |
| 14.8 | 6.0 | 1428.0 | 13.0 |
| 15.0 | 5.9 | 5113.0 | 46.7 |
| 16.4 | 5.4 | 1192.0 | 10.9 |
| 17.9 | 5.0 | 5277.0 | 48.2 |
| 18.1 | 4.9 | 1556.0 | 14.2 |
| 18.3 | 4.8 | 1167.0 | 10.7 |
| 19.2 | 4.6 | 1896.0 | 17.3 |
| 19.4 | 4.6 | 1022.0 | 9.3 |
| 20.0 | 4.4 | 1639.0 | 15.0 |
| 20.8 | 4.3 | 487.0 | 4.4 |
| 22.2 | 4.0 | 1403.0 | 12.8 |
| 22.7 | 3.9 | 10949.0 | 100.0 |
| 23.1 | 3.8 | 5844.0 | 53.4 |
| 24.0 | 3.7 | 1586.0 | 14.5 |
| 24.4 | 3.6 | 1890.0 | 17.3 |
| 25.1 | 3.5 | 926.0 | 8.5 |
| 25.7 | 3.5 | 1071.0 | 9.8 |
| 25.9 | 3.4 | 1737.0 | 15.9 |
| 26.2 | 3.4 | 2067.0 | 18.9 |
| 26.6 | 3.3 | 1283.0 | 11.7 |
| 27.1 | 3.3 | 634.0 | 5.8 |
| 29.1 | 3.1 | 760.0 | 6.9 |
| 29.7 | 3.0 | 1668.0 | 15.2 |

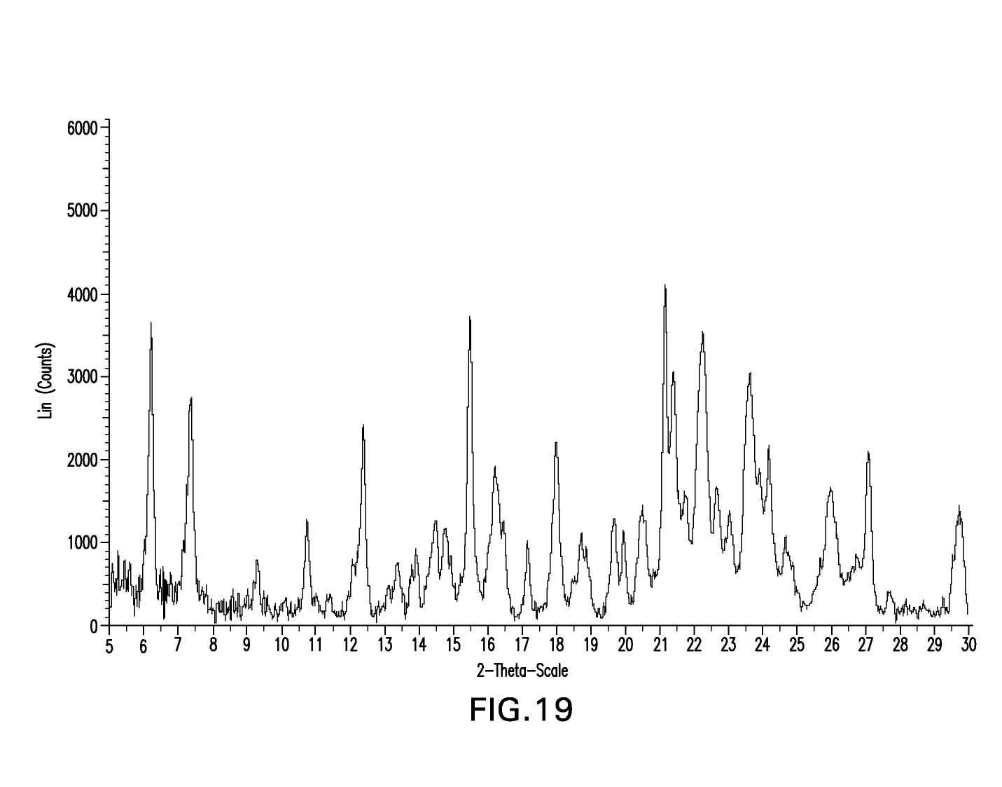

The present invention provides another different crystalline polymorph of N-(3-(dimethylamino)propyl)-4-(4-(3-fluoro-4-methoxyphenyl)pyrimidin-2-ylamino)benzenesulfonamide as an acetate salt, referred to herein as Form Ill. The Form III polymorph is identified by one or more solid-state analytical methods. The Form III polymorph exhibits a characteristic polycrystalline XRD pattern, as shown in FIG. 19. Polycrystalline XRD data consistent with the Form III polymorph are provided in Table 9.

TABLE 9
X-RAY PEAK POSITIONS OF FORM III MONO-ACETATE OF
N-(3-(DIMETHYLAMINO)PROPYL)-4-(4-(3-FLUORO-
4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 6.2 | 14.3 | 3651.0 | 89.0 |
| 7.3 | 12.1 | 2703.0 | 65.9 |
| 9.3 | 9.5 | 778.0 | 19.0 |
| 10.7 | 8.2 | 1273.0 | 31.0 |
| 12.4 | 7.2 | 2399.0 | 58.5 |
| 13.3 | 6.6 | 740.0 | 18.0 |
| 13.9 | 6.4 | 911.0 | 22.2 |
| 14.5 | 6.1 | 1252.0 | 30.5 |
| 14.7 | 6.0 | 1150.0 | 28.0 |
| 15.5 | 5.7 | 3716.0 | 90.6 |
| 16.2 | 5.5 | 1908.0 | 46.5 |
| 16.5 | 5.4 | 1236.0 | 30.1 |
| 17.2 | 5.2 | 1003.0 | 24.5 |
| 18.0 | 4.9 | 2197.0 | 53.5 |
| 18.7 | 4.7 | 1097.0 | 26.7 |
| 19.7 | 4.5 | 1269.0 | 30.9 |
| 20.0 | 4.4 | 1131.0 | 27.6 |
| 20.5 | 4.3 | 1404.0 | 34.2 |
| 21.2 | 4.2 | 4104.0 | 100.0 |
| 21.4 | 4.1 | 3047.0 | 74.2 |
| 22.3 | 4.0 | 3532.0 | 86.1 |
| 22.7 | 3.9 | 1658.0 | 40.4 |
| 23.0 | 3.9 | 1351.0 | 32.9 |
| 23.6 | 3.8 | 3011.0 | 73.4 |
| 24.2 | 3.7 | 2149.0 | 52.4 |
| 26.0 | 3.4 | 1650.0 | 40.2 |
| 27.1 | 3.3 | 2087.0 | 50.9 |
| 29.7 | 3.0 | 1428.0 | 34.8 |

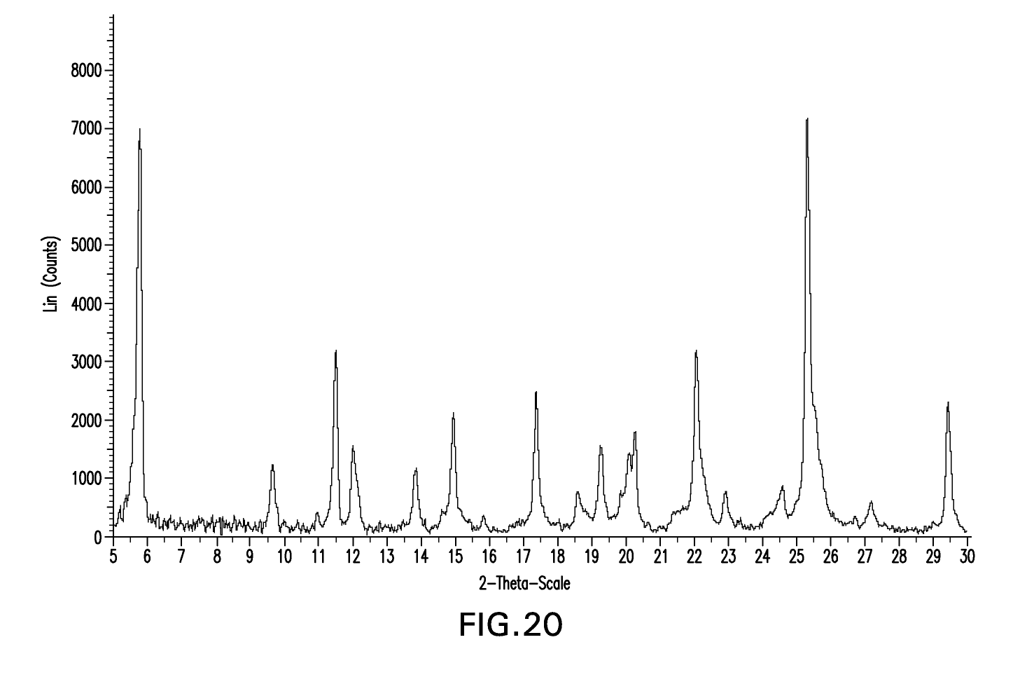

The present invention provides yet another different crystalline polymorph of N-(3-(dimethylamino)propyl)-4-(4-(3-fluoro-4-methoxyphenyl)pyrimidin-2-ylamino)benzenesulfonamide as an acetate salt, referred to herein as Form IV. The Form IV polymorph is identified by one or more solid-state analytical methods. The Form IV polymorph exhibits a characteristic polycrystalline X-ray XRD pattern, as shown in FIG. 20. Polycrystalline XRD data consistent with the Form IV polymorph are provided in Table 10.

TABLE 10
X-RAY PEAK POSITIONS OF FORM IV ACETATE OF N-(3-
(DIMETHYLAMINO)PROPYL)-4-(4-(3-FLUORO-4-
METHOXYPHENYL)-PYRIMIDIN-
2-YLAMINO)BENZENESULFONAMIDE

| Angle, 2-Theta ° | d value, Angstrom | Intensity, Count | Intensity % |
|---|---|---|---|
| 5.7 | 15.4 | 6992.0 | 97.5 |
| 8.5 | 10.4 | 328.0 | 4.6 |
| 9.6 | 9.2 | 1218.0 | 17.0 |
| 10.9 | 8.1 | 410.0 | 5.7 |
| 11.5 | 7.7 | 3174.0 | 44.2 |
| 12.0 | 7.4 | 1533.0 | 21.4 |
| 13.8 | 6.4 | 1151.0 | 16.0 |
| 14.9 | 5.9 | 2113.0 | 29.4 |
| 17.4 | 5.1 | 2471.0 | 34.4 |
| 18.6 | 4.8 | 750.0 | 10.4 |
| 19.3 | 4.6 | 1528.0 | 21.3 |
| 20.1 | 4.4 | 1415.0 | 19.7 |
| 20.3 | 4.4 | 1772.0 | 24.7 |
| 22.1 | 4.0 | 3189.0 | 44.5 |
| 22.9 | 3.9 | 770.0 | 10.7 |
| 24.6 | 3.6 | 845.0 | 11.8 |
| 25.3 | 3.5 | 7173.0 | 100.0 |
| 27.2 | 3.3 | 582.0 | 8.1 |
| 29.4 | 3.0 | 2278.0 | 31.8 |

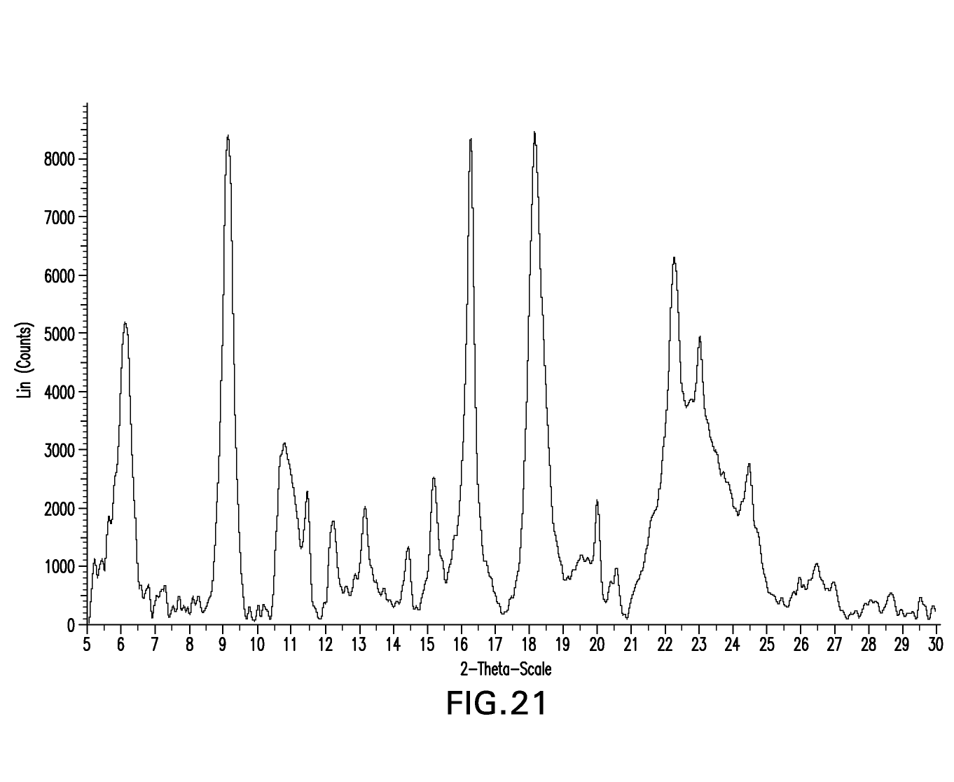

The present invention provides yet another different crystalline polymorph of N-(3-dimethylamino)propyl)-4-(4-(3-fluoro-4-methoxyphenyl)pyrimidin-2-ylamino)-benzenesulfonamide as an acetate salt, referred to herein as Form V. The Form V polymorph is identified by one or more solid-state analytical methods. The Form V polymorph exhibits a characteristic polycrystalline XRD pattern, as shown in FIG. 21. Polycrystalline XRD data consistent with the Form V polymorph is provided in Table 11 below.

TABLE 11
X-RAY PEAK POSITIONS OF FORM V MONO-ACETATE OF
N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 5.7 | 15.4 | 6992.0 | 97.5 |
| 8.5 | 10.4 | 328.0 | 4.6 |
| 9.6 | 9.2 | 1218.0 | 17.0 |
| 10.9 | 8.1 | 410.0 | 5.7 |
| 11.5 | 7.7 | 3174.0 | 44.2 |
| 12.0 | 7.4 | 1533.0 | 21.4 |
| 13.8 | 6.4 | 1151.0 | 16.0 |
| 14.9 | 5.9 | 2113.0 | 29.4 |
| 17.4 | 5.1 | 2471.0 | 34.4 |
| 18.6 | 4.8 | 750.0 | 10.4 |
| 19.3 | 4.6 | 1528.0 | 21.3 |
| 20.1 | 4.4 | 1415.0 | 19.7 |
| 20.3 | 4.4 | 1772.0 | 24.7 |
| 22.1 | 4.0 | 3189.0 | 44.5 |
| 22.9 | 3.9 | 770.0 | 10.7 |
| 24.6 | 3.6 | 845.0 | 11.8 |
| 25.3 | 3.5 | 7173.0 | 100.0 |
| 27.2 | 3.3 | 582.0 | 8.1 |
| 29.4 | 3.0 | 2278.0 | 31.8 |

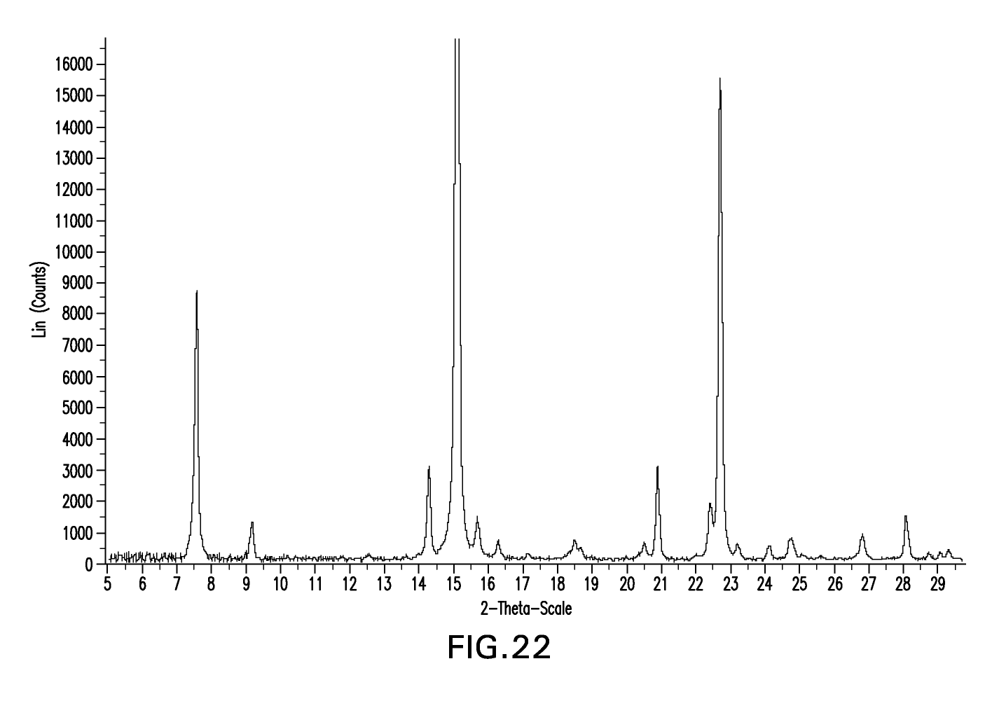

The present invention provides yet another different crystalline polymorph of N-(3-dimethylamino)propyl)-4-(4-(3-fluoro-4-methoxyphenyl)pyrimidin-2-ylamino) benzenesulfonamide as an acetate salt, referred to herein as Form VI. The Form VI polymorph is identified by one or more solid-state analytical methods. The Form VI polymorph exhibits a characteristic polycrystalline XRD pattern, as shown in FIG. 22. Polycrystalline XRD data consistent with the Form VI polymorph are provided in Table 12 below.

TABLE 12
X-RAY PEAK POSITIONS OF FORM VI MONO-ACETATE
OF N-(3-(DIMETHYLAMINO)PROPYL)-4-(4-(3-FLUORO-4-
METHOXYPHENYL)PYRIMIDIN-2-
YLAMINO)BENZENESULFONAMIDE

| Angle, 2-Theta ° | d value, Angstrom | Intensity, Count | Intensity % |
|---|---|---|---|
| 7.5 | 11.7 | 8735.0 | 23.0 |
| 9.1 | 9.7 | 1265.0 | 3.3 |
| 14.3 | 6.2 | 3073.0 | 8.1 |
| 15.1 | 5.9 | 37986.0 | 100.0 |
| 15.7 | 5.6 | 1486.0 | 3.9 |
| 16.3 | 5.4 | 709.0 | 1.9 |
| 18.5 | 4.8 | 727.0 | 1.9 |
| 20.5 | 4.3 | 643.0 | 1.7 |
| 20.9 | 4.2 | 3064.0 | 8.1 |
| 22.4 | 4.0 | 1888.0 | 5.0 |
| 22.7 | 3.9 | 15583.0 | 41.0 |
| 23.2 | 3.8 | 602.0 | 1.6 |
| 24.1 | 3.7 | 531.0 | 1.4 |
| 24.8 | 3.6 | 749.0 | 2.0 |
| 26.9 | 3.3 | 907.0 | 2.4 |
| 28.1 | 3.2 | 1496.0 | 3.9 |
| 29.3 | 3.0 | 425.0 | 1.1 |

One advantage of the mono-D-glucoronate salt is its relatively high water solubility, 5.17 mg/mL, as compared to acetate (3.5 mg/mL) and succinate (1.1 mg/mL) salts of compound 3. Preparations of crystalline polymorphs Form I-IV are provided in the Examples. In general, Form I can be prepared by dissolving compound 3 (any form, including amorphous) in a suitable solvent containing water and small amounts of solvent (e.g. isopropanol), precipitating the crystalline polymorph product from the solvent by any of numerous routine methods in the art such as by cooling or evaporating the solvent to induce precipitation. Suitable solvents include but are not limited to for example, water, a mixture of water and an alcohol, water and an ether, water mixture with conventional organic solvents and any suitable organic solvents. Water content of the solvent appears to influence the relative amounts of the Form I polymorph and the Form II polymorph, which precipitate from the mixture. Higher amounts of water in the solvent tend to favor the Form I polymorph, while lower amounts of water tend to favor the Form II polymorph. The Form II polymorph is converted back to the Form I polymorph by recrystallizing the Form II polymorph in acetone. The Form II polymorph is also used to prepare a different polymorph by adding it to TBME and recrsytallizing to a Form III polymorph. The Form II polymorph is also used to prepare yet another different crystalline polymorph, a Form IV polymorph, by recrystallizing the Form II polymorph in isopropanol, converting the Form II polymorph to the Form IV polymorph.

In an exemplary embodiment, a weight ratio, based on molar weight, of D-glucoronic acid to a free base such as compound 3 is between 1:1 to 1:5. The solvent system also appears to play a role in converting of one polymorph to other polymorphs.

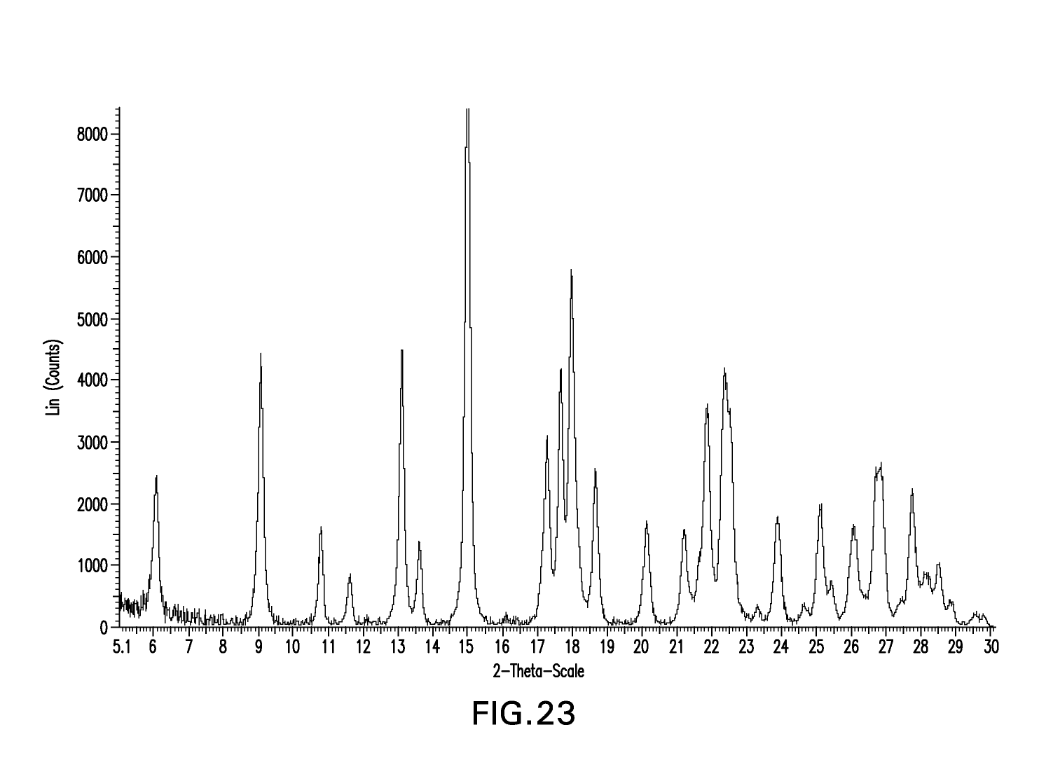

According to one embodiment, four crystalline polymorphs of compound 3 were isolated and characterized as D-glucoronate salts. The present invention provides one crystalline polymorph of N-(3-(dimethylamino)propyl)-4-(4-(3-fluoro-4-methoxyphenyl)pyrimidin-2-ylamino)benzenesulfonamide as a D-glucoronate, referred to herein as Form I. The Form I polymorph is identified by one or more solid-state analytical methods. The Form I polymorph exhibits a characteristic polycrystalline X-ray diffraction (XRD) pattern, as shown in FIG. 23. Polycrystalline XRD data consistent with the Form I polymorph are provided in Table 13 below.

TABLE 13
X-RAY PEAK POSITIONS OF FORM I D-GLUCORONATE
OF N-(3-(DIMETHYLAMINO)PROPYL)-4-(4-(3-FLUORO-
4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 6.0 | 14.6 | 2448.0 | 25.4 |
| 7.9 | 11.1 | 169.0 | 1.8 |
| 9.1 | 9.8 | 4433.0 | 46.0 |
| 10.8 | 8.2 | 1602.0 | 16.6 |
| 11.6 | 7.6 | 840.0 | 8.7 |
| 13.1 | 6.7 | 4475.0 | 46.4 |
| 13.6 | 6.5 | 1365.0 | 14.2 |
| 15.0 | 5.9 | 9642.0 | 100.0 |
| 17.3 | 5.1 | 3095.0 | 32.1 |
| 17.7 | 5.0 | 4142.0 | 43.0 |
| 18.0 | 4.9 | 5797.0 | 60.1 |
| 18.7 | 4.7 | 2551.0 | 26.5 |
| 20.2 | 4.4 | 1687.0 | 17.5 |
| 21.2 | 4.2 | 1556.0 | 16.1 |
| 21.9 | 4.1 | 3607.0 | 37.4 |
| 22.4 | 4.0 | 4193.0 | 43.5 |
| 22.6 | 3.9 | 3521.0 | 36.5 |
| 23.4 | 3.8 | 343.0 | 3.6 |
| 23.9 | 3.7 | 1763.0 | 18.3 |
| 24.7 | 3.6 | 337.0 | 3.5 |
| 25.1 | 3.5 | 1883.0 | 19.5 |
| 25.5 | 3.5 | 700.0 | 7.3 |
| 26.1 | 3.4 | 1626.0 | 16.9 |
| 26.8 | 3.3 | 2530.0 | 26.2 |
| 26.9 | 3.3 | 2629.0 | 27.3 |
| 27.8 | 3.2 | 2211.0 | 22.9 |
| 28.5 | 3.1 | 1021.0 | 10.6 |
| 28.9 | 3.1 | 402.0 | 4.2 |
| 29.7 | 3.0 | 214.0 | 2.2 |

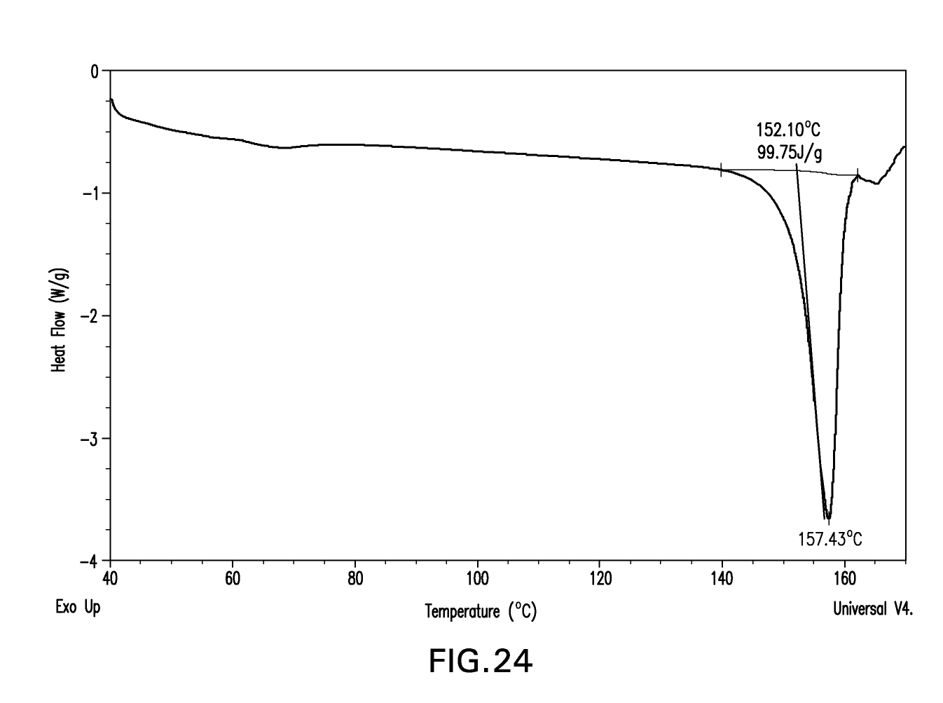

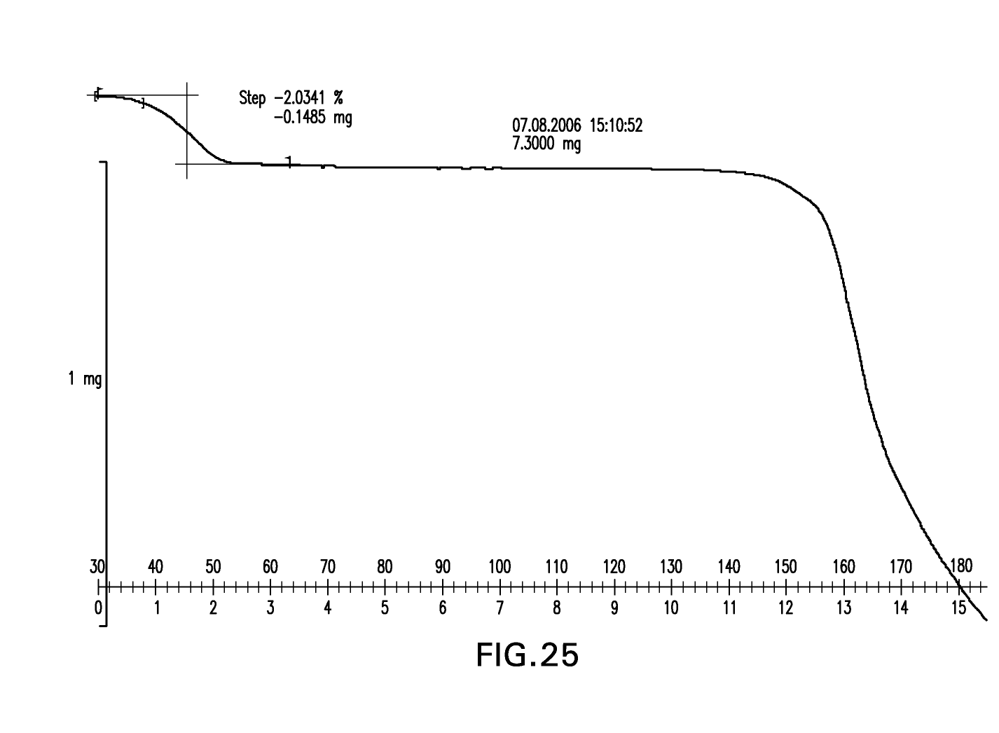

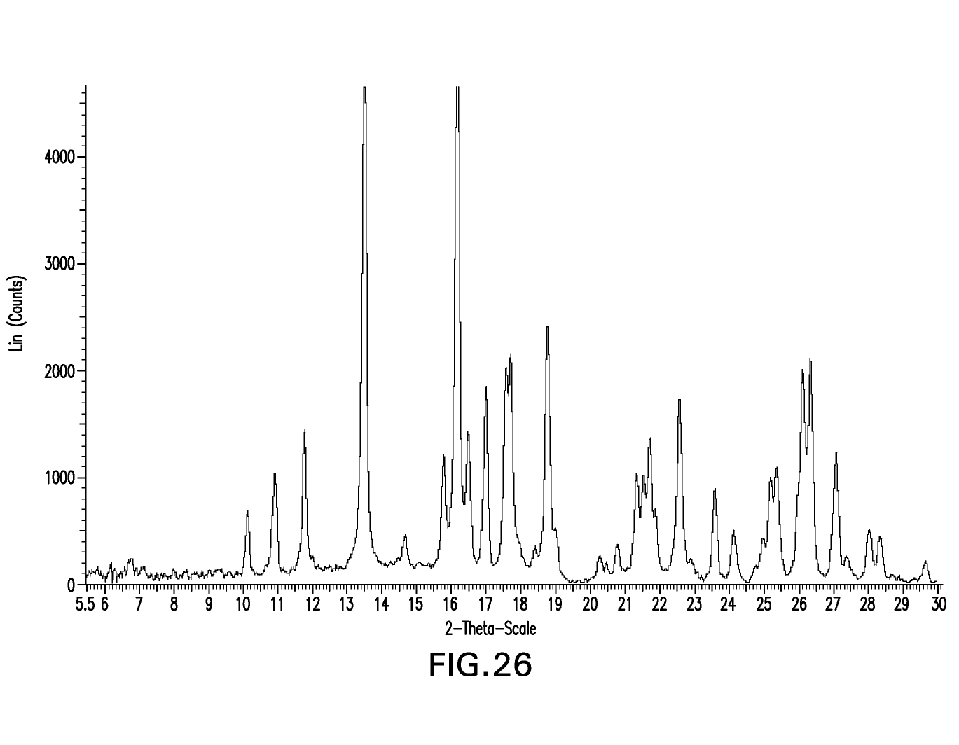

The present invention provides another crystalline polymorph of N-(3-(dimethylamino)propyl)-4-(4-(3-fluoro-4-methoxyphenyl)pyrimidin-2-ylamino)-benzenesulfonamide as a mono-D-glucuronate, referred to herein as Form II. The Form II polymorph is identified by one or more solid-state analytical methods. N-(3-(dimethylamino)propyl)-4-(4-(3-fluoro-4-methoxyphenyl)-pyrimidin-2-ylamino)-benzenesulfonamide as a D-glucoronate salt, is also identified by its characteristic differential scanning (DSC) trace, as shown in FIG. 24. Melting point is observed at 152.1° C., with an endotherm maxima at 157.4° C. N-(3-(dimethylamino)propyl)-4-(4-(3-fluoro-4-methoxyphenyl)pyrimidin-2-ylamino)benzenesulfonamide as a D-glucoronate salt, is also identified by its characteristic thermogravimetric analysis (TGA) thermogram, as shown in FIG. 25. A weight loss consistent with glucoronic acid is observed from the TGA themogram. The Form II polymorph exhibits a characteristic polycrystalline XRD pattern, as shown in FIG. 26. Polycrystalline XRD data consistent with the Form II polymorph are provided in Table 14 below.

TABLE 14
X-RAY PEAK POSITIONS OF FORM II D-GLUCORONATE
OF N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 10.1 | 8.8 | 675 | 11.6 |
| 10.9 | 8.1 | 1031 | 17.8 |
| 11.8 | 7.5 | 1444 | 24.9 |
| 13.5 | 6.6 | 4843 | 83.5 |
| 14.7 | 6.0 | 449 | 7.7 |
| 15.8 | 5.6 | 1196 | 20.6 |
| 16.2 | 5.5 | 5800 | 100.0 |
| 16.5 | 5.4 | 1420 | 24.5 |
| 17.0 | 5.2 | 1843 | 31.8 |
| 17.6 | 5.0 | 2028 | 35.0 |
| 17.7 | 5.0 | 2146 | 37.0 |
| 18.8 | 4.7 | 2404 | 41.4 |
| 19.0 | 4.7 | 523 | 9.0 |
| 20.3 | 4.4 | 254 | 4.4 |
| 20.8 | 4.3 | 360 | 6.2 |
| 21.4 | 4.2 | 1021 | 17.6 |
| 21.6 | 4.1 | 1008 | 17.4 |
| 21.7 | 4.1 | 1358 | 23.4 |
| 21.9 | 4.1 | 688 | 11.9 |
| 22.6 | 3.9 | 1718 | 29.6 |
| 23.6 | 3.8 | 881 | 15.2 |
| 24.2 | 3.7 | 500 | 8.6 |
| 25.0 | 3.6 | 416 | 7.2 |
| 25.2 | 3.5 | 988 | 17.0 |
| 25.4 | 3.5 | 1079 | 18.6 |
| 26.2 | 3.4 | 1998 | 34.4 |
| 26.4 | 3.4 | 2107 | 36.3 |
| 27.1 | 3.3 | 1224 | 21.1 |
| 28.1 | 3.2 | 497 | 8.6 |
| 28.4 | 3.1 | 439 | 7.6 |
| 29.7 | 3.0 | 200 | 3.5 |

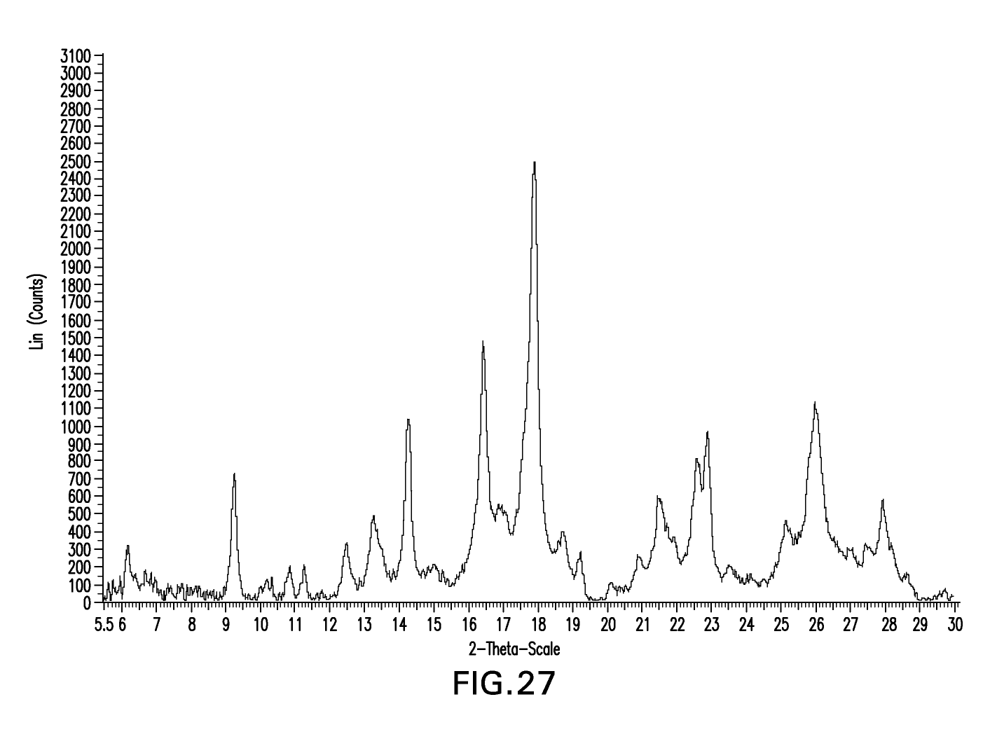

The present invention provides another crystalline polymorph of N-(3-(dimethylamino)propyl)-4-(4-(3-fluoro-4-methoxyphenyl)pyrimidin-2-ylamino)benzenesulfonamide as a D-glucuronate, referred to herein as Form III. The Form III polymorph is identified by one or more solid-state analytical methods. The Form III polymorph exhibits a characteristic powder X-ray diffraction (XRD) pattern, as shown in FIG. 27. Polycrystalline XRD data consistent with the Form III polymorph are provided in Table 15.

TABLE 15
X-RAY PEAK POSITIONS OF FORM III D-GLUCORONATE
OF N-(3-(DIMETHYLAMINO)PROPYL)-4-(4-(3-
FLUORO-4-METHOXYPHENYL)-
PYRIMIDIN-2-YLAMINO)BENZENESULFONAMIDE

| Angle, 2-Theta ° | d value, Angstrom | Intensity, Count | Intensity % |
|---|---|---|---|
| 6.1 | 14.4 | 273 | 11.2 |
| 9.2 | 9.6 | 636 | 26.2 |
| 10.2 | 8.6 | 105 | 4.3 |
| 10.8 | 8.2 | 162 | 6.7 |
| 11.2 | 7.9 | 156 | 6.4 |
| 12.5 | 7.1 | 297 | 12.2 |
| 13.3 | 6.7 | 460 | 18.9 |
| 14.2 | 6.2 | 965 | 39.7 |
| 16.4 | 5.4 | 1365 | 56.2 |
| 17.9 | 5.0 | 2429 | 100.0 |
| 18.7 | 4.7 | 376 | 15.5 |
| 19.2 | 4.6 | 248 | 10.2 |
| 20.1 | 4.4 | 97.1 | 4.0 |
| 20.9 | 4.2 | 244 | 10.0 |
| 21.5 | 4.1 | 580 | 23.9 |
| 22.6 | 3.9 | 777 | 32.0 |
| 22.9 | 3.9 | 900 | 37.1 |
| 25.2 | 3.5 | 428 | 17.6 |
| 26.0 | 3.4 | 1086 | 44.7 |
| 28.0 | 3.2 | 528 | 21.7 |

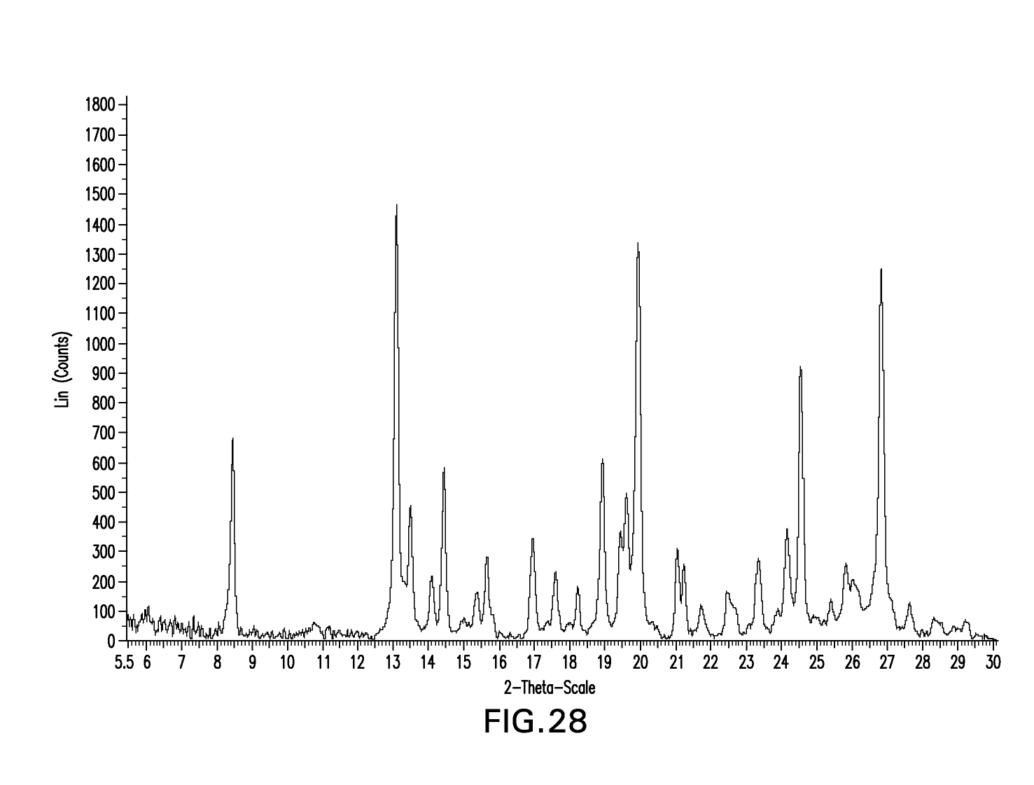

The present invention provides yet another crystalline polymorph of N-(3-(dimethylamino)propyl)-4-(4-(3-fluoro-4-methoxyphenyl)pyrimidin-2-ylamino)benzenesulfonamide as a D-glucuronate, referred to herein as Form IV. The Form IV polymorph is identified by one or more solid-state analytical methods. The Form IV polymorph exhibits a characteristic polycrystalline XRD pattern, as shown in FIG. 28. Polycrystalline XRD data consistent with the Form IV polymorph are provided in Table 16.

TABLE 16
X-RAY PEAK POSITIONS OF FORM IV MONO-D-
GLUCORONATE OF N-(3-(DIMETHYLAMINO)PROPYL)-
4-(4-(3-FLUORO-4-METHOXYPHENYL)-PYRIMIDIN-2-
YLAMINO)BENZENESULFONAMIDE

| Angle, 2-Theta ° | d value, Angstrom | Intensity, Count | Intensity % |
|---|---|---|---|
| 8.4 | 10.5 | 677.0 | 46.2 |
| 13.1 | 6.8 | 1465.0 | 100.0 |
| 13.5 | 6.6 | 451.0 | 30.8 |
| 14.1 | 6.3 | 214.0 | 14.6 |
| 14.4 | 6.1 | 580.0 | 39.6 |
| 15.4 | 5.8 | 161.0 | 11.0 |
| 15.7 | 5.7 | 278.0 | 19.0 |
| 17.0 | 5.2 | 340.0 | 23.2 |
| 17.6 | 5.0 | 226.0 | 15.4 |
| 18.2 | 4.9 | 175.0 | 12.0 |
| 18.9 | 4.7 | 607.0 | 41.4 |
| 19.5 | 4.6 | 362.0 | 24.7 |
| 19.6 | 4.5 | 491.0 | 33.6 |
| 20.0 | 4.4 | 1338.0 | 91.3 |
| 21.1 | 4.2 | 305.0 | 20.8 |
| 21.3 | 4.2 | 251.0 | 17.1 |
| 21.7 | 4.1 | 115.0 | 7.8 |
| 22.5 | 3.9 | 160.0 | 10.9 |
| 23.4 | 3.8 | 274.0 | 18.7 |
| 24.2 | 3.7 | 373.0 | 25.4 |
| 24.6 | 3.6 | 920.0 | 62.8 |
| 25.9 | 3.4 | 256.0 | 17.5 |
| 26.9 | 3.3 | 1250.0 | 85.4 |

In another embodiment, the present invention also provides pharmaceutical compositions comprising one or more crystalline forms and polymorphs of the compounds of formula I, II or III as pharmaceutically acceptable salts and a pharmaceutically acceptable carrier. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. The invention also includes pharmaceutical compositions utilizing one or more of the present compositions, compounds or polymorphs along with one or more pharmaceutically acceptable carriers, excipients, and like agents.

Formulations including one or more crystalline forms and polymorphs of compounds of formula I, II or III as pharmaceutically acceptable salts, include one or more pharmaceutically acceptable additives or carriers. Methods for preparing formulations including one or more crystalline forms and polymorphs of compounds of formula I, II or III as pharmaceutically acceptable salts include the steps of preparing the compound as a crystalline salt, including polymorphs thereof, and formulating the salts with one or more pharmaceutically acceptable additives or carriers. Formulations of the invention provide effective amounts of a composition of the invention. Daily doses may range from about 0.1 mg to about 1000 mg for a person in need. Dose ranges may vary from about 10 mg/day to about 600 mg/day, including from 10 mg/day to about 60 mg/day. The dosing can be either in a single dose or two or more divided doses per day. Such doses can be administered in any manner that facilitates the compound's entry into the bloodstream including orally, via implants, parenterally (including intravenous, intraperitoneal, and subcutaneous injection), vaginally, rectally, and transdermally.

In some embodiments, formulations including salts and polymorphs of the invention are prepared and manufactured for administering transdermally, which includes all methods of administration across the surface of the body and the inner linings of body passages including epithelial and mucosal tissues. Such administering in certain embodiments includes, but is not limited to for example, a foam, a patch, a suspension, or a solution.

Oral formulations containing the salts and polymorphs of this invention can comprise any conventionally used oral forms, including tablets, capsules, buccal forms, troches, lozenges and oral liquids, suspensions or solutions. According to an exemplary embodiment, capsules may contain mixtures of one or more crystalline polymorphs in the desired percentage together with any other polymorph(s) of N-(3-(dimethylamino)propyl)-4-(4-(3-fluoro-4-methoxyphenyl)pyrimidin-2-ylamino)-benzenesulfonamide as a pharmaceutically acceptable salt or any structurally related compounds. Capsules or tablets of the desired crystalline form of the desired percentage composition may also be combined with mixtures of other active compounds or inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, and the like.

Tablet formulations can be additionally prepared and manufactured by conventional compression, wet granulation, or dry granulation methods and utilize pharmaceutically acceptable diluents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein can utilize standard delay or time-release formulations or spansules. Suppository formulations can be made from traditional materials, including cocoa butter, with or without the addition of waxes to alter the suppositories melting point, and glycerin. Water-soluble suppository bases, such as polyethylene glycols of various molecular weights, can also be used.

Excipient systems suitable for preparing formulations of the present inveted salts and polymorphs thereof include one or more fillers, disintegrants, and lubricants.

The filler component can be any filler component known in the art including, but not limited to, lactose, microcrystalline cellulose, sucrose, mannitol, calcium phosphate, calcium carbonate, powdered cellulose, maltodextrin, sorbitol, starch, or xylitol.

Disintegrants suitable for use in the present formulations of the invented salts and polymorphs thereof can be selected from those known in the art, including pregelatinized starch and sodium starch glycolate. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein can comprise from about 4% to about 40% of the composition by weight, preferably from about 15% to about 35%, more preferably from about 20% to about 35%.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel™), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit™), potassium chloride, powdered cellulose, sodium chloride, sorbitol and talc.

Solid pharmaceutical compositions of the salts and polymorphs thereof are compacted into a dosage form, such as a tablet, which may include excipients whose functions include helping to bind the active ingredient and other excipients together after compression. Binders for solid pharmaceutical compositions include acacia, alginic acid, carbomer (e.g. Carbopol™), carboxymethylcellulose sodium, dextrin, ethyl cellulose, gelatin, guar gum, hydrogenated vegetable oil, hydroxyethyl cellulose, hydroxypropyl cellulose (e.g. Klucel™), hydroxypropyl methyl cellulose (e.g. Methocel™), liquid glucose, magnesium aluminum silicate, maltodextrin, methylcellulose, polymethacrylates, povidone (e.g. Kollidon™, Plasdone™), pregelatinized starch, sodium alginate, starch and others known in the art.

The dissolution rate of a compacted solid pharmaceutical composition including the salts and polymorphs thereof in the patient's stomach may be increased by the addition of a disintegrant to the composition. Disintegrants include alginic acid, carboxymethylcellulose calcium, carboxymethylcellulose sodium (e.g. Ac-Di-Sol™, Primellose™), colloidal silicon dioxide, croscarmellose sodium, crospovidone (e.g. Kollidon™, Polyplasdone™), guar gum, magnesium aluminum silicate, methyl cellulose, microcrystalline cellulose, polacrilin potassium, powdered cellulose, pregelatinized starch, sodium alginate, sodium starch glycolate (e.g. Explotab™), starch and others known in the art.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions including salts and polymorphs of the present invention, the compound of formula I and any other solid excipients are dissolved or suspended in a liquid carrier such as water, vegetable oil, alcohol, polyethylene glycol, propylene glycol or glycerin.

Liquid pharmaceutical compositions including salts and polymorphs of the invention may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl-cellulose, carbomer, cetostearyl alcohol and cetyl alcohol.

Liquid pharmaceutical compositions including salts and polymorphs of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

The pharmaceutical formulations of the salts and polymorphs thereof and other structurally related compounds can also contain an antioxidant or a mixture of antioxidants, such as ascorbic acid. Other useful antioxidants include, but are not limited to for example, sodium ascorbate and ascorbyl palmitate, preferably in conjunction with an amount of ascorbic acid. An example range for the antioxidant(s) is from about 0.5% to about 15% by weight, most preferably from about 0.5% to about 5% by weight.

The formulations of the salts and polymorphs thereof described herein can be used in an uncoated or non-encapsulated solid form. In some embodiments, the pharmacological compositions are optionally coated with a film coating, for example, comprising from about 0.3% to about 8% by weight of the overall composition. Film coatings useful with the present formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. The compositions and formulations herein may also be combined and processed as a solid, then placed in a capsule form, such as a gelatin capsule.

Pharmaceutical compositions of the salts and polymorphs thereof and other structurally related compounds and other structurally related compounds can be formulated with steroidal estrogens, such as conjugated estrogens. The amount used in the formulation can be adjusted according to the particular polymorph form or ratio of polymorph forms used, the amount and type of steroidal estrogen in the formulation as well as the particular therapeutic indication being considered. In general, the polymorphic composition ratio can be used in an amount sufficient to antagonize the effect of the particular estrogen to the level desired. The dose range of conjugated estrogens can be from about 0.3 mg to about 2.5 mg, about 0.3 mg to about 1.25 mg, or about 0.3 mg to about 0.625 mg. An example range for amount of N-(3-dimethylamino)propyl)-4-(4-(3-fluoro-4-methoxyphenyl)pyrimidin-2-ylamino)benzenesulfonamide as a mono-succinate salt or hemi-succinate salts in a combination formulation is about 10 mg to about 40 mg. For the steroidal estrogen mestranol, for example, a daily dosage can be from about 1 μg to about 150 μg, and for ethynyl estradiol a daily dosage of from about 1 μg to 300 μg can be used. In some embodiments, the daily dose is between about 2 μg and about 150 μg.

According to the present invention, a liquid composition including salts and polymorphs of the invention may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions including salts and polymorphs of the present invention include powders, granulates, aggregates and compacted compositions. The dosages include dosages suitable for oral, buccal, rectal, parenteral (including subcutaneous, intramuscular, and intravenous), inhalant and ophthalmic administration. The most suitable administration in any given case will depend on the nature and severity of the condition being treated. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, such as a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition, including salts and polymorphs of the invention, for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition, including salts and polymorphs of the invention, may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may include any of the aforementioned blends and granulates that were described with reference to tableting, however, they are not subjected to a final tableting step.

Methods of administration of a pharmaceutical composition encompassed by the invention are not specifically restricted, and can be administered in various preparations depending on the age, sex, and symptoms of the patient. For example, tablets, pills, solutions, suspensions, emulsions, granules and capsules may be orally administered. Injection preparations may be administered individually or mixed with injection transfusions such as glucose solutions and amino acid solutions intravenously. If necessary, the injection preparations are administered singly intramuscularly, intracutaneously, subcutaneously or intraperitoneally. Suppositories may be administered into the rectum.

The amount of the one or more crystalline forms and polymorphs of the compounds of formula I contained in a pharmaceutical composition according to the present invention is not specifically restricted, however, the dose should be sufficient to treat, ameliorate, or reduce the targeted symptoms. The dosage of a pharmaceutical composition according to the present invention will depend on the method of use, the age, sex, and condition of the patient.

The present invention also provides methods of inhibition and treatment further comprising administering one or more crystalline forms and polymorphs of the compounds of formula I, including free base forms of compounds of formula I, and an additional inhibitor of a protein kinase of the NF-κB pathway. Inhibitors of a protein kinase of the NF-κB pathway include, but are not limited to IKK inhibitors and GSK-3 inhibitors. IKK inhibitors include, but are not limited to heterocyclic carboxamides, substituted benzimidazoles, substituted indoles, β-carbolines such as PS-1145, SPC0023579, SPC839/AS602868 (AS2868), NVPIKK004, and NVPIKK005. GSK-3 inhibitors include, but are not limited to maleimides such as SB410111, SB495052, SB517955, SB216763, SB415286, diamino-1,2,4-triazole carboxylic acid derivatives and 2,5-dihydro-1H-pyrrole-2,5,-dione derivatives, diaminothiazoles, bicyclic compounds, pyrazine derivatives, pyrimidine- or pyridine derivatives, and purine derivatives such as CT 98014, CT98023, CT99021, 2-amino-3-(alkyl)-pyrimidone derivatives, 1H-imidazol-4-amine derivatives, and 3-indolyl-4-phenyl-1H-pyrrole-2,5-dione derivatives, as described in Haefner, B. (2002) “NF-κB: arresting a major culprit in cancer,” Drug Discovery Today, 7, 658.

The pharmaceutical compositions of the present invention may comprise one or more crystalline forms and polymorphs of the compounds of formula I or in combination with other kinase-inhibiting compounds or chemotherapeutic agents. Chemotherapeutic agents include, but are not limited to exemestane, formestane, anastrozole, letrozole, fadrozole, taxane and derivatives such as paclitaxel or docetaxel, encapsulated taxanes, CPT-11, camptothecin derivatives, anthracycline glycosides, e.g., doxorubicin, idarubicin, epirubicin, etoposide, navelbine, vinblastine, carboplatin, cisplatin, estramustine, celecoxib, tamoxifen, raloxifen, Sugen SU-5416, Sugen SU-6668, and Herceptin.

Having described the invention, the invention is further illustrated by the following non-limiting examples.

### EXAMPLES

Acquisition of analytical data. Differential scanning calorimetry data were collected on pharmaceutically acceptable salts of compounds having formulas I-III using a DSC (TA instruments, model Q1000) under the following parameters: 50 mL/min. purge gas (N₂); scan range 40 to 200° C., scan rate 10° C./min. Thermo-gravimetric analysis data was collected using a TGA instrument (Mettler Toledo, model TGA/SDTA 851e) under the following parameters: 40 mL/min. purge gas(N₂); scan range 30 to 250° C., scan rate 10° C./min. XRD data was acquired using an X-ray powder diffractometer (Bruker-axs, model D8 advance) having the following parameters: voltage 40 kV, current 40.0 mA, scan range (2θ) 5 to 300, scan step size 0.01°, total scan time 33 minutes, VANTEC detector, and anti-scattering slit 1 mm. NMR spectra were acquired on a Brucker 300 MHz spectrometer using a dueterated solvent and tetramethylsilane (TMS) as an internal standard (δ=0.0 ppm). All δ values are expressed in units of parts per million (ppm).

### Example 1

Mono-succinate salt of N-(3-(dimethylamino)propyl)-4-(4-(3-fluoro-4-methoxyphenyl)pyrimidin-2-ylamino)benzenesulfonamide. Compound 3 (1.2442 g) as a free base was poured into vial and 13.7 mL acetone:water (5:2) was added, forming a slurry. The slurry of compound 3 was heated to ˜50-55° C. Succinic acid (382 mg) was added. The mixture was stirred for 10 minutes and 2.5 mL acetone was added. The mixture was cooled to room temperature and 12 mL acetone was added and the mixture stirred for 30 minutes, then filtered and dried at 50° C. under vacuum to form a water soluble pharmaceutically acceptable salt. The salt was characterized as a mono-succinate salt of compound 3 by a combination of analytical techniques. DSC analysis indicated the melting point of the mono-succinate salt is 171° C. Analysis of NMR data indicated the ratio of succinate to compound 3 was 1:1. TGA indicated a weight loss consistent with succinic acid. X-ray diffraction data for the crystalline form as a mono-succinate salt was summarized in Table 1. Free base: ¹H NMR (d₆-DMSO, 300 MHz): 61.97 (s), 2.04 (s), 2.08 (s), 2.14 (m), 2.26 (m), 2.52 (m), 2.62 (br s), 2.77 (t). Mono-succinate salt of 3: ¹H NMR (d₆-DMSO, 300 MHz): δ2.23 (s), 2.29 (m), 2.38 (m), 2.51 (m), 2.78 (t).

### Example 2

Mono-succinate salt of N-(3-(dimethylamino)propyl)-4-(4-(3-fluoro-4-methoxyphenyl)pyrimidin-2-ylamino)benzenesulfonamide. 2a) Compound 3 (454 mg) was poured into a vial and 5 mL of water was added, forming a slurry. Succinic acid (122 mg) was added and the slurry was heated to 50° C. until all ingredients dissolved. The mixture was cooled to room temperature with stirring to afford the mono-succinate salt. The salt was filtered and dired at 50° C. under vacuum. 2b) Alternatively, an ethanol:water mixture was added to a 4:1 ratio by weight of compound 3:succinic acid. The slurry was heated to 55° C., cooled and the salt was filtered and dried at 50° under vacuum. In both cases, XRD data for the crystalline mono-succinate salt of 3 was confirmed by comparing XRD to that summarized in Table 1.

### Example 3

Form I Polymorph of the Hemi-succinate salt of N-(3-(dimethylamino)propyl)-4-(4-(3-fluoro-4-methoxyphenyl)pyrimidin-2-ylamino)benzenesulfonamide. Compound 3 (0.5199 g) free base was poured into vial and 5.7 mL acetone:water (5:2) was added, forming a slurry. The slurry of compound 3 was heated to about 50-55° C. Succinic acid (100.6 mg) was added. The mixture was stirred for 10 minutes and 2.5 mL acetone was added. The mixture was cooled to room temperature and 3 mL acetone was added and the mixture stirred for 30 minutes, then filtered and dried at 50° C. under vacuum to form a water soluble pharmaceutically acceptable salt. The hemi-succinate salt of compound 3 was characterized by a combination of analytical techniques. DSC analysis indicates the melting point of the hemi-succinate is 125° C. Analysis of the NMR data indicates the ratio of succinate to compound 3 was 2:1. TGA indicated a weight loss consistent with succinic acid. XRD data for the one crystalline polymorph (Form I) was summarized in Table 2. Hemi-succinate salt of 3: ¹H NMR (d₆-DMSO, 300 MHz): δ2.07 (s), 2.09 (m), 2.15 (s), 2.28 (m), 2.36 (s), 2.51 (m), 2.78 (t).

### Example 4

Form II Polymorph of the Hemi-succinate salt of N-(3-(dimethylamino)propyl)-4-(4-(3-fluoro-4-methoxyphenyl)pyrimidin-2-ylamino)-benzenesulfonamide. Compound 3 (111.3 mg) free base was poured into vial and 1.2 mL acetone:water (5:2) was added, forming a slurry. The slurry of compound 3, in a reduced volume of acetone:water as compared to Example 3, was heated to about 60° C. Succinic acid (21.7 mg) was added. The mixture was cooled to room temperature and the Form II polymorph precipitated in pure form. The Form II salt was then filtered and dried at 50° C. under vacuum to form a water soluble pharmaceutically acceptable salt. The Form II polymorph of the hemi-succinate salt of compound 3 was characterized by a combination of analytical techniques, including XRD, DSC and TGA. XRD data for the crystalline form was summarized in Table 3.

### Example 5

Form III Polymorph of the Hemi-succinate salt of N-(3-(dimethylamino)propyl)-4-(4-(3-fluoro-4-methoxyphenyl)pyrimidin-2-ylamino)benzenesulfonamide. Compound 3 (5 mg) free base was poured into a DSC crucible, which was heated to about 70° C. using a heating rate of 10 C/minute and after heating was cooled to room temperature. The Form III polymorph as the hemi-succinate salt of compound 3 was characterized by XRD data and was summarized in Table 4.

### Example 6

Form IV Polymorph of the Hemi-succinate salt of N-(3-(dimethylamino)propyl)-4-(4-(3-fluoro-4-methoxyphenyl)pyrimidin-2-ylamino)-benzenesulfonamide. Compound 3 (5 mg) free base was poured into a DSC crucible, which was heated to about 125° C. using a heating rate of 10 C/minute and after heating was cooled to room temperature. The Form IV polymorph as the hemi-succinate salt of compound 3 was characterized by XRD data for the crystalline polymorph, summarized in Table 5.

### Example 7

Form V Polymorph of the Hemi-succinate salt of N-(3-(dimethylamino)propyl)-4-(4-(3-fluoro-4-methoxyphenyl)pyrimidin-2-ylamino)-benzenesulfonamide. Compound 3 (5 mg) free base was poured into a DSC crucible, which was heated to about 140° C. using a heating rate of 10° C./minute and after heating was cooled to room temperature. The Form V polymorph as the hemi-succinate salt of compound 3 was characterized by XRD data for the crystalline polymorph, summarized in Table 6.

### Example 8

Form I Polymorph of the acetate salt of N-(3-(dimethylamino)propyl)-4-(4-(3-fluoro-4-methoxyphenyl)pyrimidin-2-ylamino)-benzenesulfonamide. Compound 3 (52.7 mg) as a free base was poured into vial and 0.53 mL tetrahydrofuran (THF) was added, forming a slurry. The slurry of compound 3 was heated to ˜50-55° C. Acetic acid (6.88 μL) was added. The mixture was stirred for 10 minutes and 2.5 mL acetone was added. The mixture was stirred for 1 hr then filtered and dried at 50° C. under vacuum to form a water soluble pharmaceutically acceptable mono-acetate salt. The mono-acetate salt of compound 3 was characterized by a combination of analytical techniques. The melting point of the acetate salt is 129° C. from the endotherm indicated by DSC analysis. The ratio of acetate to compound 3 was determined to be 1:1 from XRD, DSC and TGA. TGA indicated a weight loss consistent with acetic acid. XRD data for the crystalline form was summarized in Table 7.

### Example 9

Form II Polymorph of the acetate salt of N-(3-(dimethylamino)propyl)-4-(4-(3-fluoro-4-methoxyphenyl)pyrimidin-2-ylamino)-benzenesulfonamide. Compound 3 (832.2 mg) as a free base was poured into vial and 17 mL of ethyl acetate was added at room temperature, forming a slurry. Acetic acid (110 μL) was added. The mixture was stirred for 48 hr then filtered and dried at 50° C. under vacuum to form crystals of a water-soluble pharmaceutically acceptable mono-acetate salt. The acetate salt of compound 3 was characterized by a combination of analytical techniques. The melting point of the acetate salt is 129° C. from the endotherm indicated by DSC analysis. The ratio of acetate to compound 3 was determined to be 1:1. TGA indicated a weight loss consistent with acetic acid. XRD data for the crystalline polymorph was summarized in Table 8. Alternatively, in 2.58 mL of ethyl acetate and heating the slurry to 60° C., the Form II polymorph was produced as a crystalline acetate salt, that was filtered and dried under vacuum. XRD data was consistent with the Form II polymorph.

### Example 10

Form III Polymorph of the acetate salt of N-(3-(dimethylamino)propyl)-4-(4-(3-fluoro-4-methoxyphenyl)pyrimidin-2-ylamino)-benzenesulfonamide. Compound 3 (105 mg) as a free base was poured into vial and 2.35 mL of isopropanol was added at room temperature, forming a slurry. The slurry was heated to 60-65° C. to dissolve the solids. Acetic acid (13.7 μL) was added. The mixture was stirred for 1 hr at 25° C., then filtered and dried at 50° C. under vacuum to form crystals of a water soluble pharmaceutically acceptable salt. The acetate salt of compound 3 was characterized by a combination of analytical techniques. The melting point of the acetate salt is 129° C. from the endotherm indicated by DSC analysis. The ratio of acetate to compound 3 was determined to be 1:1. TGA indicated a weight loss consistent with acetic acid. XRD data for the crystalline polymorph was summarized in Table 9.

### Example 11

Form IV Polymorph of the acetate salt of N-(3-(dimethylamino)propyl)-4-(4-(3-fluoro-4-methoxyphenyl)pyrimidin-2-ylamino)-benzenesulfonamide. The Form II polymorph as the mono-acetate salt of compound 3 was slurried in acetone at 25° C. for 2 days, precipitating formed crystals of the water-soluble pharmaceutically acceptable mono-acetate salt. The mono-acetate salt of compound 3 was characterized by a combination of analytical techniques. The ratio of acetate to compound 3 was determined to be 1:1. TGA indicated a weight loss consistent with acetic acid. XRD data for the crystalline polymorph was summarized in Table 10.

### Example 12

Form V Polymorph of the acetate salt of N-(3-(dimethylamino)propyl)-4-(4-(3-fluoro-4-methoxyphenyl)pyrimidin-2-ylamino)-benzenesulfonamide. The Form II polymorph as the acetate salt of compound 3 was slurried and diluted by 4 volumes of water at 25° C. for 1 day, precipitating formed crystals of the water-soluble pharmaceutically acceptable mono-acetate salt. The acetate salt of compound 3 was characterized by a combination of analytical techniques. The ratio of acetate to compound 3 was determined to be 1:1. TGA indicated a weight loss consistent with acetic acid. XRD data for the crystalline polymorph was summarized in Table 11.

### Example 13

Form VI Polymorph of the acetate salt of N-(3-(dimethylamino)propyl)-4-(4-(3-fluoro-4-methoxyphenyl)pyrimidin-2-ylamino)-benzenesulfonamide. Form II of the acetate salt of Compound 3 was slurried in a mixture of ethanol:methanol (100:10) at 25° C. for 2 days, precipitating formed crystals of the water-soluble pharmaceutically acceptable acetate salt. The acetate salt of compound 3 was characterized by a combination of analytical techniques. The ratio of acetate to compound 3 was determined to be 1:1. TGA indicated a weight loss consistent with acetic acid. XRD data for the crystalline polymorph was summarized in Table 12.

### Example 14

Form I Polymorph of the D-glucoronate salt of N-(3-(dimethylamino)propyl)-4-(4-(3-fluoro-4-methoxyphenyl)pyrimidin-2-ylamino)-benzenesulfonamide. The free base compound 3 (65.9 mg) was poured into vial and 0.66 mL water was added at room temperature. The solution of compound 3 was combined with D-glucoronic acid (29.5 mg) and 0.77 mL of isoproponal was added. The mixture was stirred for 1 hour and then allowed to form a crystalline precipitate. The D-glucoronate salt of compound 3 was characterized by a combination of analytical techniques. The melting point of the D-glucoronate salt of compound 3 is 152° C. from the endotherm indicated by DSC analysis. The ratio of D-glucoronate to compound 3 was determined to be 1:1. TGA indicated a weight loss consistent with glucoronic acid. XRD data for the crystalline form is summarized in Table 13.

### Example 15

Form II Polymorph of D-glucoronate salt of N-(3-(dimethylamino)propyl)-4-(4-(3-fluoro-4-methoxyphenyl)pyrimidin-2-ylamino)benzene-sulfonamide. The free base compound 3 (857.9 mg) was poured into vial and 6.86 mL water was added at room temperature. The solution of compound 3 was combined with D-glucoronic acid (378 mg) and 0.30 mL of water and 6.56 mL isopropanol was added. The mixture was stirred for 48 hours and then allowed to form a crystalline precipitate. The crystals were filtered and dried at 50° C. under vacuum. The D-glucoronate salt of compound 3 was characterized by a combination of analytical techniques. The melting point of the D-glucoronate salt of compound 3 is 152° C. from the endotherm indicated by DSC analysis. The ratio of D-glucoronate to compound 3 was determined to be 1:1. TGA indicated a weight loss consistent with glucoronic acid. XRD data for the crystalline Form II polymorph is summarized in Table 14.

### Example 16

Conversion of Form II Polymorph of the D-glucoronate salt of N-(3-(dimethylamino)propyl)-4-(4-(3-fluoro-4-methoxyphenyl)-pyrimidin-2-ylamino)-benzene-sulfonamide to Form I Polymorph of D-glucoronate salt of N-(3-(dimethylamino)propyl)-4-(4-(3-fluoro-4-methoxyphenyl)-pyrimidin-2-ylamino)-benzene-sulfonamide. The Form II polymorph of compound 3 as the D-glucoronate salt was slurried in acetone at room temperature for 2 days to produce the Form I polymorph of compound 3 as a D-glucoronate salt, as confirmed by comparing XRD data with the Form I polymorph in Table 13.

### Example 17

Form III Polymorph of the D-glucoronate salt of N-(3-(dimethylamino)propyl)-4-(4-(3-fluoro-4-methoxyphenyl)pyrimidin-2-ylamino)-benzenesulfonamide. The Form II polymorph of compound 3 as a D-glucoronate salt was slurried in t-BME at room temperature for 4 days and then allowed to form a crystalline precipitate. The crystals were filtered and dried at 50° C. under vacuum. The D-glucoronate salt of compound 3 was characterized by a combination of analytical techniques. XRD data for the crystalline Form III polymorph is summarized in Table 15.

### Example 18

Form IV Polymorph of D-glucoronate salt of N-(3-(dimethylamino)propyl)-4-(4-(3-fluoro-4-methoxyphenyl)pyrimidin-2-ylamino)-benzenesulfonamide. The Form II polymorph of compound 3 as a D-glucoronate salt was slurried in isopropanol at room temperature for 4 days and then allowed to form a crystalline precipitate. The crystals were filtered and dried at 50° C. under vacuum. The D-glucuronate salt of compound 3 was characterized by a combination of analytical techniques. XRD data for the crystalline Form IV polymorph is summarized in Table 16.

What is claimed is:

1. A crystalline form or polymorph of a compound of formula I: 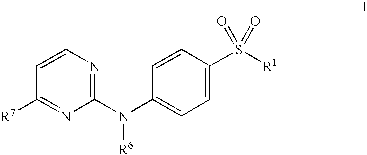
in the form of a pharmaceutically acceptable salt; wherein, R¹ is —NR²R³, wherein R² and R³ are independently selected from the group consisting of: C₁-C₅ substituted alkyl, C₂-C₅ substituted alkenyl, C₂-C₅ substituted alkynyl, C₂-C₅ substituted aryl, C₁-C₅ substituted heteroaryl, hydroxyl, C₁-C₅ substituted alkoxy, C₁-C₅ substituted alkylamino, C₁-C₅ substituted arylamino, C₁-C₅ substituted heteroarylamino, —NCOR⁴, —COR⁴, —CONR²R³, SO₂R⁵, C₄-C₁₀ substituted 3 to 10 membered cyclic amines containing 0 to 3 heteroatoms; R⁴ and R⁵ are independently selected from the group consisting of hydrogen, methyl, trifluoromethyl, substituted alkyl, substituted aryl, and substituted heteroaryl; R⁶ is selected from the group consisting of hydrogen, C₁-C₅ substituted alkyl, C₁-C₅ substituted alkylcarbonyl, and C₁-C₅ substituted alkoxycarbonyl; and wherein R⁷ is a substituted phenyl selected from the group consisting of: 2,4-substituted phenyl, 3,4-substituted phenyl, 4,5-substituted phenyl and 4,6-substituted phenyl, the substituted phenyl having substituents selected from the group consisting of: C₁-C₅ alkyl, F, Cl, Br, I, C₁-C₅ alkoxy, C₁-C₅ alkylamine, C₁-C₅ alkylamino, C₁-C₅ amide, C₁-C₅ ester, hydroxy, and C₁-C₅ alkyl-, C₁-C₅ alkoxy-, C₁-C₅ alkylamino-substituted amides, NH₂, trifluoromethyl, C₁-C₅ substituted alkyl trifluoromethyl, and phenyl.

2. The crystalline form or polymorph of the compound of formula I of claim 1, wherein R² is NH₂, -(dimethylamino)ethyl, or -(dimethylamino)propyl and R⁵ is hydrogen or methyl.

3. The crystalline form or polymorph of the compound of formula I of claim 1, wherein R⁷ is a 3,4-substituted phenyl or a 4,5-substituted phenyl having substituents selected from the group consisting of: C₁-C₅ substituted alkoxy, F, Cl, Br and I.

4. The crystalline form or polymorph of the compound of formula I of claim 3, further comprising a pharmaceutically acceptable salt selected from the group consisting of: succinate, D-glucoronate, L-glucoronate, acetate, oxalate, proprionate, maleate, benzoate and citrate.

5. The crystalline form or polymorph of the compound of formula I of claim 1, comprising N-(3-(dimethylamino)propyl)-4-(4-(3-fluoro-4-methoxyphenyl)pyrimidin-2-ylamino)benzene sulfonamide as a pharmaceutically acceptable salt selected from the group consisting of: succinate, D-glucoronate, L-glucoronate, acetate, oxalate, proprionate, maleate, benzoate and citrate.

6. The crystalline form or polymorph of the compound of formula I of claim 1 comprising a mono-succinate salt.

7. The crystalline form or polymorph of the compound of formula I of claim 6, characterized by X-ray diffraction peaks at the following angles (±0.2°) of 2θ in its X-ray diffraction pattern: 10.9°, 11.4°, 13.1°, 15.2°, 17.1°, 17.9°, 21.0°, 21.9°, 22.9° and 24.4°.

8. The crystalline form or polymorph of the compound of formula I of claim 6, characterized by NMR signals at δ values of: 2.23 ppm, 2.29 ppm, 2.38 ppm, 2.51 ppm, and 2.78 ppm.

9. The crystalline form or polymorph of the compound of formula I of claim 1 comprising a hemi-succinate salt.

10. The crystalline form or polymorph of the compound of formula I of claim 9 characterized by NMR signals at δ values of: 2.07 ppm, 2.09 ppm, 2.15 ppm, 2.28 ppm, 2.36 ppm, 2.51 ppm, and 2.78 ppm.

11. The crystalline form or polymorph of the compound of formula I of claim 9 characterized by X-ray diffraction peaks at the following angles (±0.2°) of 2θ in its X-ray diffraction pattern: 6.7°, 13.2°, 14.0°, 14.6°, 19.0°, 19.9°, 20.1°, 21.2°, 21.5°, 22.6°, 23.2°, 25.3°, 25.6° and 28.1°.

12. The crystalline form or polymorph of the compound of formula I of claim 9 characterized by X-ray diffraction peaks at the following angles (±0.2°) of 2θ in its X-ray diffraction pattern: 7.0°, 10.7°, 11.2°, 12.8°, 13.2°, 13.8°, 14.1°, 15.0°, 16.3°, 17.3°, 17.7°, 18.0°, 18.4°, 20.6°, 23.3°, 23.5°, 26.4°, 26.6° and 28.5°.

13. The crystalline form or polymorph of the compound of formula I of claim 9 characterized by X-ray diffraction peaks at the following angles (±0.2°) of 2θ in its X-ray diffraction pattern: 10.9°, 11.7°, 12.3°, 13.5°, 14.7°, 15.1°, 16.2°, 17.0°, 17.3°, 17.7°, 18.6°, 25.9° and 26.3°.

14. The crystalline form or polymorph of the compound of formula I of claim 9 characterized by X-ray diffraction peaks at the following angles (±0.2°) of 2θ in its X-ray diffraction pattern: 5.2°, 6.5°, 7.2°, 7.6°, 11.0°, 12.1°, 14.2°, 14.6°, 15.4°, 16.6°, 17.3°, 17.6°, 18.6°, 21.4°, 21.6°, 21.9°, 22.9°, 23.3°, 25.7° and 27.0°.

15. The crystalline form or polymorph of the compound of formula I of claim 9 characterized by X-ray diffraction peaks at the following angles (±0.2°) of 2θ in its X-ray diffraction pattern: 5.2°, 7.1°, 9.2°, 10.3°, 14.1°, 14.4°, 15.4°, 16.2°, 16.4°, 17.5°, 18.1°, 18.5°, 21.3°, 22.7°, 25.5° and 27.0°.

16. The crystalline form or polymorph of the compound of formula I of claim 1 comprising an acetate salt.

17. The crystalline form or polymorph of the compound of formula I of claim 16, characterized by X-ray diffraction peaks at the following angles (±0.2°) of 2θ in its X-ray diffraction pattern: 7.3°, 12.1°, 15.4°, 16.7°, 17.8°, 18.3°, 22.1°, 22.4°, 24.1°, 26.2°, 26.4°, 27.3° and 28.0°.

18. The crystalline form or polymorph of the compound of formula I of claim 16, characterized by X-ray diffraction peaks at the following angles (±0.2°) of 2θ in its X-ray diffraction pattern: 7.4°, 10.7°, 12.5°, 15.0°, 17.9°, 19.2°, 20.0°, 22.7°, 23.1°, 24.4°, 25.9°, 26.2° and 29.7°.

19. The crystalline form or polymorph of the compound of formula I of claim 16, characterized by X-ray diffraction peaks at the following angles (±0.2°) of 2θ in its X-ray diffraction pattern: 6.2°, 7.3°, 9.3°, 10.7°, 12.4°, 13.3°, 13.9°, 14.5°, 14.7°, 15.5°, 16.2°, 16.5°, 17.2°, 18.0°, 18.7°, 19.7°, 20.0°, 20.5°, 21.2°, 21.4°, 22.3°, 22.7°, 23.0°, 23.6°, 24.2°, 26.0°, 27.1° and 29.7°.

20. The crystalline form or polymorph of the compound of formula I of claim 16, characterized by X-ray diffraction peaks at the following angles (±0.2°) of 2θ in its X-ray diffraction pattern: 5.7°, 9.6°, 11.5°, 12.0°, 13.8°, 14.9°, 17.4°, 19.3°, 20.3°, 22.1°, 25.3° and 29.4°.

21. The crystalline form or polymorph of the compound of formula I of claim 16, characterized by X-ray diffraction peaks at the following angles (±0.3°) of 2θ in its X-ray diffraction pattern: 7.5°, 15.1°, 20.9° and 22.7°.

22. The crystalline form or polymorph of the compound of formula I of claim 16 characterized by X-ray diffraction peaks at the following angles (±0.2°) of 2θ in its X-ray diffraction pattern: 5.7°, 9.6°, 11.5°, 12.0°, 13.8°, 14.9°, 17.4°, 18.6°, 19.3°, 20.1°, 20.3°, 22.1°, 22.9°, 24.6°, 25.3° and 29.4°.

23. The crystalline form or polymorph of the compound of formula I of claim 1 comprising a D-glucoronate salt.

24. The crystalline form or polymorph of the compound of formula I of claim 23, characterized by X-ray diffraction peaks at the following angles (±0.2°) of 2θ in its X-ray diffraction pattern: 6.0°, 9.1°, 10.8°, 13.1°, 13.6°, 15.0°, 17.3°, 17.7°, 18.0°, 18.7°, 20.2°, 21.2°, 21.9°, 22.4°, 22.6, 23.9, 25.1°, 26.1°, 26.8°, 26.9°, 27.8° and 28.5°.

25. The crystalline form or polymorph of the compound of formula I of claim 23, characterized by X-ray diffraction peaks at the following angles (±0.2°) of 2θ in its X-ray diffraction pattern: 10.1°, 10.9°, 11.8°, 13.5°, 15.8°, 16.2°, 16.5°, 17.0°, 17.6°, 18.8°, 21.4°, 21.6°, 21.7°, 22.6°, 23.6, 25.2, 26.2°, 26.4° and 27.1°.

26. The crystalline form or polymorph of the compound of formula I of claim 23, characterized by X-ray diffraction peaks at the following angles (±0.2°) of 2θ in its X-ray diffraction pattern: 6.1°, 9.2°, 12.5°, 13.3°, 14.2°, 16.4°, 17.9°, 18.7°, 21.5°, 22.6°, 22.9°, 25.2°, 26.0° and 28.0°.

27. The crystalline form or polymorph of the compound of formula I of claim 23, characterized by X-ray diffraction peaks at the following angles (±0.2°) of 2θ in its X-ray diffraction pattern: 8.4°, 13.1°, 13.5°, 14.1°, 14.4°, 15.7°, 17.0°, 17.6°, 18.2°, 19.5°, 19.6°, 20.0°, 21.1°, 21.3°, 22.5, 23.4, 24.2°, 24.6°, 25.9° and 26.9°.

28. A crystalline form of a compound of formula:

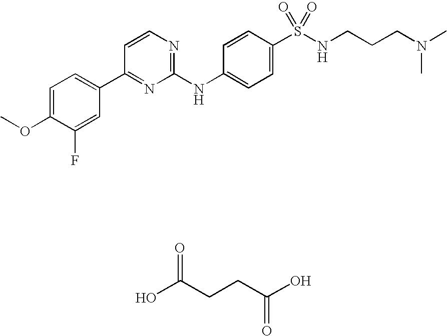

29. A crystalline polymorph of a compound of formula:

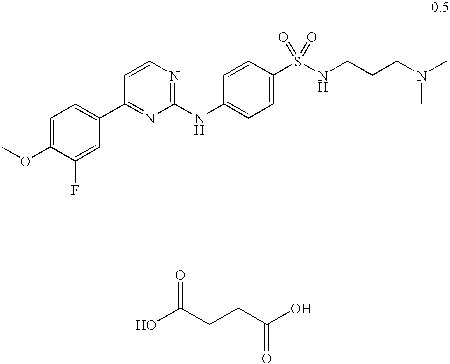

30. A crystalline polymorph of a compound of formula:

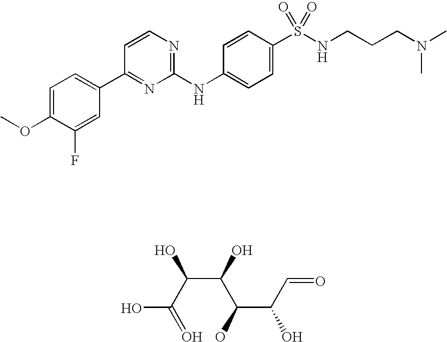

31. A crystalline polymorph of a compound of formula:

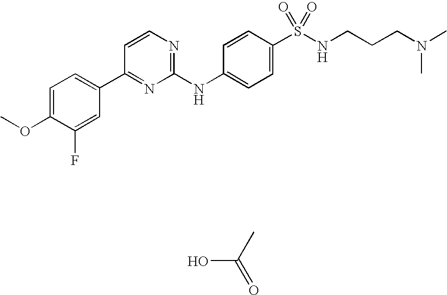

32. A pharmaceutical composition comprising: one or more crystalline forms or polymorphs of the compound of formula I as pharmaceutically acceptable salts of claim 1, or in combination with other kinase-inhibiting pharmaceutical compositions or chemotherapeutic agents, and a pharmaceutically acceptable carrier.

33. A pharmaceutical composition comprising one or more crystalline polymorphs of the compound of formula I as pharmaceutically acceptable salts of claim 5, or in combination with other kinase-inhibiting pharmaceutical compositions or chemotherapeutic agents, and a pharmaceutically acceptable carrier.

34. A method of inhibiting kinase activity in a mammal comprising administering to a mammal a kinase-inhibiting amount of one or more crystalline polymorphs of the compound of formula I of claim 1.

35. The method of claim 34, wherein the mammal is a human.

36. A method of treating a kinase-dependent condition comprising administering to a subject a kinase-inhibiting amount of a pharmaceutical composition according to claim 32.
